(19) United States
(12) Patent Application Publication
(10) Pub. No.: US 2019/0310172 A1
(43) Pub. Date: Oct. 10, 2019

(54) PROFILING EXTRACELLULAR VESICLES

(71) Applicant: Caris Science, Inc., Irving, TX (US)

(72) Inventors: Zhenyu ZHONG, Peoria, AZ (US); David SPETZLER, Paradise Valley, AZ (US)

(21) Appl. No.: 16/376,851

(22) Filed: Apr. 5, 2019

Related U.S. Application Data
(60) Provisional application No. 62/653,445, filed on Apr. 5, 2018.

(51) Int. Cl. G01N 1/40; G01N 33/68; C12N 15/10

(52) U.S. Cl. CPC G01N 1/4077; C12N 15/1048; G01N 33/6848

(57) ABSTRACT

Methods and compositions are provided for enriching extracellular vesicles, e.g., from bodily fluids. The vesicles may be indicative of various diseases. Vesicle characterization can be used for diagnostic, theranostic and other purposes.

### CROSS REFERENCE

This application claims the benefit of U.S. Provisional Patent Application No. 62/653,445, filed Apr. 5, 2018; which application is incorporated herein by reference in its entirety.

### TECHNICAL FIELD

The present disclosure relates to the field of biomarker isolation and analysis, namely isolation and analysis of extracellular vesicles.

### BACKGROUND

Extracellular vesicles (EV; also referred to as microvesicles, exosomes, etc.) are found in bodily fluids and provide a source material to examine biomarker profiles, e.g., for use in liquid biopsies or other diagnostics purposes. Development of a fast and reliable preparation protocol to enrich such small particles could accelerate the use of such EVs in clinical settings, e.g., to detect or characterize disease, and the discovery of informative, disease-related biomarkers. Although multiple EV enrichment protocols are available, in terms of efficiency, reproducibility and simplicity, precipitation based methods are advantageous for laboratory testing or for studies with large numbers of subjects. For example, such methods may be more rapid and less equipment intensive than classical ultracentrifugation (UC) based methods. However, the selectivity of the precipitation process can vary, e.g., some methods are more selective in enriching or isolating EVs apart from abundant proteins or other biological entities.

Here we provide an EV enrichment protocol based on pluronic block copolymer. The enriched EVs were characterized using multiple platforms. Using human plasma as an exemplary bodily fluid, we found that the particles enriched from plasma by the copolymer were EV sized vesicles with the expected membrane structure, and are significantly enriched in known EV proteins and nucleic acids. At the same time, the EVs have significantly lower amounts of high abundant plasma proteins in comparison to other precipitation based enrichment methods. Next generation sequencing (NGS) confirmed the existence of various RNA species that have been observed in EVs from previous studies. In summary, we have developed a plasma EV enrichment method with improved precipitation selectivity and suitable for larger scale studies.

### INCORPORATION BY REFERENCE

All publications, patents and patent applications mentioned in this specification are herein incorporated by reference to the same extent as if each individual publication, patent or patent application was specifically and individually indicated to be incorporated by reference.

### SUMMARY

In an aspect, the invention provides a method of isolating at least one extracellular vesicle from a sample comprising: (a) contacting the sample with at least one pluronic block copolymer; and (b) separating extracellular vesicles associated with the pluronic block copolymers from one or more components of the sample thereby isolating the at least one extracellular vesicle. Any useful pluronic block copolymer can be used. In some embodiments, the at least one pluronic block copolymer comprises F68, F127 or HLB0.80. Other useful pluronic block copolymers, or poloxamers, are commercially available. In some preferred embodiments, the pluronic block copolymer comprises F68. See Example 3 herein.

As described herein, many species of extracellular vesicles have been identified, which may be generally referred to herein as microvesicles or vesicles. See, e.g., Table 2. In some embodiments, the at least one extracellular vesicle comprises vesicles having a diameter between 10 nm and 1000 nm. In some embodiments, the at least one extracellular vesicle comprises vesicles having a diameter between 20 nm and 200 nm. The at least one extracellular vesicle may comprise vesicles having a diameter between 20 nm and 100 nm.

As desired, alternative purification techniques can be combined with the methods provided herein. For example, the at least one extracellular vesicle can be subjected to affinity purification, filtration, polymer precipitation, PEG precipitation, ultracentrifugation, a molecular crowding reagent, affinity isolation, affinity selection, or any combination thereof. As a non-limiting example, consider that a sample can be filtered or centrifuged to clarify large particulates from the sample, after which the sample can be contacted with the pluronic block copolymers to isolate extracellular vesicles.

The sample contacted with the pluronic block copolymer can be any useful sample, e.g., suspected or known to comprise extracellular vesicles. Extracellular vesicles have been found in most, if not all, bodily fluids and are shed from many types of cells. In some embodiments, the sample comprises a bodily fluid, tissue sample or cell culture. The bodily fluid can be any useful source of extracellular vesicles, including without limitation peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair oil, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood. In preferred embodiments, the sample comprises peripheral blood, serum or plasma.

The methods provided herein may further comprise selectively depleting at least one contaminant from the sample prior to step (a), between steps (a) and (b), after step (b), or any combination thereof. A contaminant can be any undesirable entity within the sample, e.g., an entity that can interfere with downstream analysis of the extracellular vesicles. For example, the at least one contaminant can include at least one highly abundant protein, such as albumin or IgG in blood.

The at least one extracellular vesicle isolated according to the methods provided herein can be used for downstream analysis. See, e.g., Example 3 herein (demonstrating a variety of different techniques for proteomic and nucleic acid profiling of the isolated extracellular vesicles). In some embodiments, the methods comprise detecting at least one surface antigen associated with the isolated at least one extracellular vesicle. Any useful and desired surface antigen can be detected. In some embodiments, the at least one surface antigen is selected from Table 3 or Table 6. As further described herein, vesicle surface antigens can be indicative of the origin of the vesicles (e.g., the type of tissue or cell that shed the vesicles), or may comprise markers of disease or other biological states. In some embodiments, the methods provided herein further comprise contacting the isolated at least one extracellular vesicle with at least one binding agent, wherein the at least one binding agent comprises a nucleic acid, DNA molecule, RNA molecule, antibody, antibody fragment, aptamer, peptoid, zDNA, peptide nucleic acid (PNA), locked nucleic acid (LNA), lectin, peptide, dendrimer, membrane protein labeling agent, chemical compound, or a combination thereof. In preferred embodiments, the at least one binding agent comprises an antibody and/or an aptamer. As a non-limiting example, the binding agent can be directed to a surface antigen of interest. Such binding agents can be used to capture and/or detect vesicles. See, e.g., FIGS. 1A-E herein and related discussion.

In some embodiments, the isolated extracellular vesicles are assessed using an aptamer library. Such assessment may comprise contacting the isolated at least one extracellular vesicle with an aptamer library prior to step (a), between steps (a) and (b), after step (b), or any combination thereof; and identifying the members of the aptamer library that bound the isolated at least one extracellular vesicle. Such identifying may comprise any useful techniques, e.g., sequencing, hybridization or amplification. As a non-limiting example, high throughput sequencing, or next-generation sequencing (NGS), methods can be used to detect and quantify the aptamers that bound the vesicles. See, e.g., FIGS. 2A-F and related discussion.

The methods provided herein may further comprise detecting at least one payload biomarker within the isolated extracellular vesicles. Vesicles are known to carry various biological entities (e.g., proteins, nucleic acids) within their lumen. In some embodiments, the one or more payload biomarker comprises at least one nucleic acid, peptide, protein, lipid, antigen, carbohydrate, and/or proteoglycan. The nucleic acid may include at least one of DNA, RNA, mRNA, smRNA, microRNA, Y RNA, lincRNA, mitochondrial RNA, snoRNA, snRNA, rRNA, tRNA, siRNA, hnRNA, or shRNA. See available nucleic acids in FIGS. 6A-B. In some embodiments, the nucleic acid comprises microRNA. In some embodiments, the nucleic acid comprises mRNA. As one non-limiting example, consider that a population of extracellular vesicles is isolated according to the methods herein, and then a microRNA profile from the isolated vesicles is determined.

In some embodiments, the sample contacted with the pluronic block co-polymer is from a subject suspected of having or being predisposed to a disease or disorder. As noted herein, extracellular vesicles and associated biomarkers (e.g., surface antigens and/or payload) can be used for the detection or characterization (e.g., diagnosis, prognosis and/or theranosis) of various diseases and disorders. As a non-limiting example, consider that a cancer cell sheds a population of microvesicles. As the surface content and payload of the vesicles are derived from the cancer cell, the vesicles provide a means of surveying the cancer. In some instances, the extracellular vesicles are shed into circulation and thus provide a useful means for liquid biopsy. In various embodiments, the disease or disorder comprises a cancer, a premalignant condition, an inflammatory disease, an immune disease, an autoimmune disease or disorder, a cardiovascular disease or disorder, neurological disease or disorder, infectious disease, pain, or any combination thereof See, e.g., Table 1.

In some preferred embodiments, the isolated at least one extracellular vesicle is used to characterize a cancer. The vesicle itself may be a marker of the cancer. In some embodiments, a presence or level of the isolated at least one extracellular vesicle is compared to a reference in order to characterize the cancer. Any useful reference can be used, e.g., a level of vesicles in a subject without the cancer or with a different stage of the cancer. In some embodiments, a biomarker associated with the isolated at least one extracellular vesicle is compared to a reference in order to characterize the cancer. Various vesicle associated biomarkers are disclosed herein. For example, the biomarker may be at least one protein in Table 3 or 6. The vesicle biomarker can be a nucleic acid, e.g., mRNA or microRNA. The characterizing may comprise providing a prognostic, diagnostic or theranostic determination for the cancer, e.g., identifying the presence or risk of the cancer, or identifying the cancer as metastatic or aggressive.

As cancer cells are known to shed microvesicles, any cancer can be characterized by the methods provided herein, including without limitation acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; lung cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom macroglobulinemia; or Wilm's tumor. In some embodiments, the cancer comprises breast cancer.

In a related aspect, the invention provides a kit comprising at least one reagent for carrying out the methods provided herein. In preferred embodiments, the at least one reagent comprises at least one reagent for isolating extracellular vesicles. For example, the at least one reagent can be F68, F127, or HLB0.80. The at least one reagent may comprise a column, tube or other reagent to remove contaminants from the sample or otherwise provide desired sample pre- or post-processing. As a non-limiting example, affinity columns to abundant blood proteins may be included in the kit. The at least one reagent can also be configured for downstream analysis of the isolated vesicles, including without limitation at least one of an aptamer library pre-enriched to bind a vesicle population of interest; dyes or other labels, e.g., to detect the vesicles; a primer, set of primer, or probes to detect nucleic acids, e.g., nucleic acid payload such as RNA within the vesicles; antibodies or aptamers to at least one vesicle surface antigen, e.g., antigens in Table 3 or 6; a column, tube, plate, bead, particle or other substrate configured to capture a vesicle (see, e.g., FIGS. 1A-1B and 2D); and any useful combination thereof.

In another aspect, the invention provides a method of detecting an extracellular vesicle in a sample, comprising contacting the extracellular vesicle with at least one binding agent to at least one protein listed in Table 6. The extracellular vesicle may be isolated according to the methods provided herein, e.g. as described above. Any useful binding agent to the protein can be chosen. In some embodiments, the at least one binding agent comprises a nucleic acid, DNA molecule, RNA molecule, antibody, antibody fragment, aptamer, peptoid, zDNA, peptide nucleic acid (PNA), locked nucleic acid (LNA), lectin, peptide, dendrimer, membrane protein labeling agent, chemical compound, or a combination thereof. In preferred embodiments, the binding agent may comprise an antibody or aptamer. For example, the binding agent can be used for capture or detection of the vesicle as shown in FIGS. 1A-1B or described elsewhere herein. In some embodiments, the sample is from a subject suspected of having or being predisposed to a disease or disorder. The disease or disorder can be, e.g., a cancer, a premalignant condition, an inflammatory disease, an immune disease, an autoimmune disease or disorder, a cardiovascular disease or disorder, neurological disease or disorder, infectious disease, pain, or any combination thereof. In some embodiments, the presence or level of the detected at least one extracellular vesicle is compared to a reference in order to characterize the cancer. In addition or in the alternative, a biomarker associated with the detected at least one extracellular vesicle can be compared to a reference in order to characterize the cancer. Such characterization may include providing a prognostic, diagnostic or theranostic determination for the cancer, identifying the presence or risk of the cancer, or identifying the cancer as metastatic or aggressive. The cancer can be a cancer described herein. In some embodiments, the cancer comprises breast cancer.

### BRIEF DESCRIPTION OF THE DRAWINGS

FIGS. 1A-1E illustrate methods of assessing biomarkers such as microvesicle surface antigens. FIG. 1A is a schematic of a planar substrate coated with a capture agent, such as an aptamer or antibody, which captures vesicles expressing the target antigen of the capture agent. The capture agent may bind a protein expressed on the surface of vesicles shed from diseased cells (“disease vesicle”). The detection agent, which may also be an aptamer or antibody, carries a detectable label, here a fluorescent signal. The detection agent binds to the captured vesicle and provides a detectable signal via its fluorescent label. The detection agent can detect an antigen that is generally associated with vesicles, or is associated with a cell-of-origin or a disease, e.g., a cancer. FIG. 1B is a schematic of a particle bead conjugated with a capture agent, which captures vesicles expressing the target antigen of the capture agent. The capture agent may bind a protein expressed on the surface of vesicles shed from diseased cells (“disease vesicle”). The detection agent, which may also be an aptamer or antibody, carries a detectable label, here a fluorescent signal. The detection agent binds to the captured vesicle and provides a detectable signal via its fluorescent label. The detection agent can detect an antigen that is generally associated with vesicles, or is associated with a cell-of-origin or a disease, e.g., a cancer. FIGS. 1C-1D present illustrative schemes for capturing and detecting vesicles to characterize a phenotype. FIG. 1E presents illustrative schemes for assessing vesicle payload to characterize a phenotype.

FIGS. 2A-F illustrate development and use of an oligonucleotide probe library, or aptamer library, to distinguish biological sample types.

FIGS. 3A-J. Characterization of the EVs Enriched By Pluronic Copolymer F68: FIG. 3A) DLS characterization of the particle size: i) cell line EV, ii) cell line EV in PBS precipitated by F68, iii) cell line EV in plasma enriched by F68, iv) native plasma EV enriched by F68, v) PBS buffer control. FIG. 3B) TEM image of EVs from: i) the cell line EVs, ii) cell line EV in PBS precipitated by F68, iii) plasma with cell line EV spike-in precipitated by F68, iv) native plasma EVs enriched by F68 with arrow point to the label of the EV size. All scale of the images are 100 nm as shown by the scale bar on lower left corner of the images. FIG. 3C) CD9 Immuno-gold TEM, EVs from i) cell line EVs, ii) plasma spiked with cell line EVs and precipitated by F68, and iii) native plasma EV enriched by F68, were labeled with anti-CD9 antibody followed by secondary antibody with gold particle. FIG. 3D) Direct CD9 ELISA, 1 μg of proteins from each sample were used, two replicate per sample (*significantly higher than corresponding plasma sample, p<0.05). FIG. 3E) smRNA recovery analysis: gel image from bioanalyzer for comparing smRNA recovery efficiency, all samples were processed in the same way, equal volume of the eluted sample (1μ1) were analyzed. Arrow point to the three most concentrated bands for density analysis; FIG. 3F) smRNA recovery analysis: density analysis of three distinct cell line EV smRNA bands (140 bp, 90 bp and 60 bp). Plots comparison including in density from individual band and combined density from all three bands are shown. FIG. 3G) Relative abundance (AACt analysis) of let-7a microRNA from EV fraction enriched by F68 method compared to original plasma, 5 replicate for each sample (plasma as reference sample, miR-451 as reference miR; *significantly higher than corresponding plasma sample, p<0.05). FIG. 3H) Protein recovery by F68 precipitation with cell line EVs in PBS. FIG. 3I) Flow Cytometry: EVs enriched by F68 from plasma spiked with cell line EV; iv-vi) Native plasma EVs enriched by F68; i,iv) samples without labeling with antibody; ii,v) samples labeled with isotype antibody-PE antibody; iii,vi) samples were labeled with tetraspanin antibody-PE cocktail (CD9/CD63/CD81). Green line and red lines indications in i) are the same for each plot. The green line and red lines are the minimal/maximal thresholds for SALS-area and LALS-area respectively to gate the particle population. The extracted particle events are then projected in the event distribution histogram according to their PE fluorescence signal strength. FIG. 3J) left panel) Event distribution histogram for EVs enriched by F68 from plasma spiked with cell line EV; right panel) Event distribution histogram for native plasma EVs enriched by F68. (Tet+: tetraspanin CD9/CD63/CD81 antibody-PE positive population; isotype+: isotype antibody-PE positive population).

FIGS. 4A-B. Comparing Different Plasma EV Enrichment Methods: FIG. 4A) Western blot comparison of plasma high abundant protein and EV-related protein among three precipitation reagent used. Plasma high abundant proteins include serum albumin (ALB), Alpha-2-macroglobulin (A2M), plasma antibody (IgG). Plasma EV-related proteins include CD9, β-Actin (ACTB), Heat Shock 70 (HSP70) and von Willebrand factor (VWF); FIG. 4B) Same comparison of the plasma high abundant proteins and EV-related proteins by semi-quantitative shotgun proteomic analysis. (Exoq: Exoquick; TEL Total Exosome Isolation Kit, 4 replicate per sample; * significantly lower compared to original plasma (p<0.05); ** significantly lower compared to Exoquick/TEI methods (p<0.05); *** significantly enriched compared to original plasma; **** significantly enriched compared to Exoquick/TEI methods.

FIGS. 5A-E. Categorization of Plasma EVs Proteins Enriched From Different Enrichment Method: FIG. 5A) Venn Diagram of unique and shared identified protein features (in term of detectable and non-detectable) in MS analysis from F68, Exoquick, TEI kit as well as the corresponding plasma. FIG. 5B) Venn Diagram of unique and shared protein features identified from the semi-quantitative MS analysis from different EV enrichment protocols that are highly enriched from the corresponding neat plasma. The highly enriched features is defined as the average of relative abundance is significantly higher (p<0.05 by t-test) than the corresponding neat plasma by equal or over 10 folds from respective methods. FIG. 5C: protein categorization for plasma EV enriched by F68, FIG. 5D: protein categorization for plasma EV enriched by Exoquick, FIG. 5E: protein categorization for plasma EV enriched by TEL In C-E, unique protein features for individual method identified in A and B were categorized by their GO term to extracellular exosome (Exo), cytoskeleton, cell surface, endosome, blood macromolecule, endoplasmic reticulum (ER), mitochondrial, Golgi apparatus and nucleosome. Potential EV includes the proteins from EV, cytoskeleton, cell surface, and endosome as well as proteins that matched in Exocarta. Potential contaminants include blood macromolecule, endoplasmic reticulum (ER), mitochondrial, Golgi apparatus and nucleosome category. Undetermined category includes the proteins that could be identified either as potential EV and potential contaminants.

FIGS. 6A-D. NGS Profiling of Plasma EVs Enriched By Pluronic Copolymer F68: Total RNA profiling were categorized according to their biotype (www.gencodegenes.org/gencode_biotypes.html) in two aspects: FIG. 6A) Regardless of the abundancy, only summarize the species counts according to their biotypes, the frequency in the pie chart indicated proportion of transcripts species identified for each biotype; FIG. 6B) Frequency in the pie chart accounts for the combined relative expression level in term of FPKM for each biotype. FIG. 6C) Heatmap showing supervised hierarchical clustering of smRNA from plasma EV enriched by F68 and their corresponding plasma, from blue to red indicates smRNA expression level (log 2 normalized expression). FIG. 6D) Density plot of smRNAs relative abundance in comparison to plasma in terms of fold changes. Fold changes were indicated in log 2 scale, the arrow marks the smRNA set that enriched from plasma.

FIGS. 7A-F. Clinical Comparison between Advanced Breast Cancer and Non-Cancer: FIG. 7A) Age distribution in cancer and non-cancer group in Whiskers box plot. FIG. 7B) The unique proteins identified in the patient sample (protein detected at least in 10 samples in each group) were categorized to EV, cytoskeleton, cell surface, endosome, blood macromolecule, endoplasmic reticulum (ER), mitochondrial, Golgi apparatus and nucleosome. Potential EV, Potential contaminants and un-determined category are separated as described above by their GO term. FIG. 7C) Proteins overlapping between four breast cancer cell lines generated EVs and the clinical patient plasma sample EVs. FIG. 7D) Volcano plot of the cancer vs non-cancer plasma EV samples, the t-test p value (−log 10) was plotted against the fold changes of the average relative abundance between cancer and non-cancer plasma EV samples. FIG. 7E) Relative abundance of protein enriched from plasma EV comparison between cancer and non-cancer group by Whiskers box plot, as indicated: ACTB, VWF, ALB, A2M and IgG. FIG. 7F) Comparison of relative protein abundance from enriched plasma EV (blue) to the neat plasma (red) in dot plot from individual patients, as indicated: ACTB, VWF (some red dots are interspersed with the blue population), ALB, A2M and IgG. Pearson correlation between plasma EV and neat plasma were also calculated. Y axis is the log 10 transformed relative abundance.

FIGS. 8A-C. Classification Performance and Feature Selection by LOO cross-validation model: FIG. 8A) Classification performance of the semi-quantitative mass spectrometry data was evaluated by a LOO cross-validation model based on random forest. The overall performance is AUC 0.7625. FIG. 8B) Classification performance on random permutations. Distribution of the AUC performance from all permutations was shown as blue, red vertical line marks the AUC performance of the correct sample label. p value is calculated by the percentage of permutation with equal of higher AUC performance than the original AUC. FIG. 8C) Non-supervised hierarchical clustering of the patient samples by the features selected from the prediction model above. Two clusters were clearly formed in the patient samples, cluster 1 contains 16 cancers and 3 non-cancers (80% sensitivity), cluster 2 contains 4 cancer and 17 non-cancers (85% specificity).

FIGS. 9A-D. Top Features Selected comparison between advanced breast cancer/non-cancer groups as well as comparison between enriched plasma EVs and the neat plasma: Whisker Boxplot comparison between cancer/non-cancer; dot plots comparison of individual enriched plasma EV and their corresponding neat plasma (blue: plasma EV, red: neat plasma). FIG. 9A: CNOT2, ITGB3. FIG. 9B: KIAA0100, ODF2. FIG. 9C: PRSS3, REV3L. FIG. 9D: CDC14A, NKAP.

FIGS. 10A-C. Semi-quantitative mass spectrometry analysis of proteins that expected to be absence or under-presented in EVs: Comparison of relative abundance of Cytochrome c (FIG. 10A; CYCS), Calnexin (FIG. 10B; CANX) and Endoplasmin (FIG. 10C; HSP90B1). Semi-quantitative mass spectrometry analysis for number of protein markers that were supposed to be absence or under-presented in EV fraction were shown to compare between different plasma EV enrichment preparation as well as the corresponding plasma: The relative abundance of CYCS, a mitochondria related protein, showed highest level in plasma. While the relative abundance of three EV preparations were all significantly lower than that from the original plasma, EVs enriched by the F68 method showed least CYCS contamination (but not absence) among all three methods; For one endoplasmic reticulum related protein CANX, it showed that the relative abundance of CANX from all three EV plasma preparation methods are significantly lower than the corresponding plasma, while the average relative abundance of CANX from EV enriched by F68 method was slightly higher than Exoquick and slightly lower than TEI; In contrast, another endoplasmic reticulum protein HSP90B1 showed a different pattern, it showed that EV prepared by F68 method with the least contamination of HSP90B1, but it is at similar level compared to the original plasma. Both EVs fraction enriched by Exoquick and TEI showed significantly level of contaminated HSP90B1 compared to that from F68 method; Therefore, only F68 method consistently show significant lower enrichment of these mitochondria and endoplasmic reticulum protein marker in the enriched plasma EV fraction. In the figures, * indicates significant lower compared to plasma (p<0.05); ** indicates significant lower than Exoquick and TEI samples (p<0.05).

### DETAILED DESCRIPTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

Disclosed herein are compositions and methods that can be used to assess a biomarker profile, which can include a presence or level of one or more biomarkers. The compositions and methods of the invention are used to isolate, or enrich, extracellular vesicles from a biological source, such as a bodily fluid. Unless otherwise clear in context, the terms “isolation” or “enrichment” are understood herein to include removal in whole or in part of non-target entities in the biological source. In one non-limiting example, isolation or enrichment of microvesicles from blood plasma can mean that the concentration or purity of microvesicles is increased and/or the presence of other constituents in the plasma such as highly abundant proteins (albumin, antibodies, etc) is reduced.

The isolated microvesicles can be used for downstream analysis, e.g., by probing microvesicle surface antigens or a functional fragment thereof The antigens typically comprise proteins or polypeptides but can be any useful component displayed on a microvesicle surface including nucleic acids, lipids and/or carbohydrates. The methods disclosed comprise diagnostic processes and techniques using microvesicles isolated according to the invention, e.g., by determining the level or presence of relevant microvesicle surface antigens or a functional fragment thereof. In some embodiments, binding agents such as antibodies are aptamers are used to capture, isolate, or enrich, a cell, cell fragment, vesicle or any other fragment or complex that comprises the antigen or functional fragments thereof.

### METHODS

Biomarker Detection and Diagnostics

The compositions and methods provided herein can be used to assess a presence or level of biomarkers in a biological sample, e.g., biological entities of interest such as proteins, nucleic acids, or microvesicles. The biomarkers may be used to identify a biosignature. A “biosignature” as used herein refers to a biomarker profile of a biological sample comprising a presence, level or other characteristic that can be assessed (including without limitation a sequence, mutation, rearrangement, translocation, deletion, epigenetic modification, methylation, post-translational modification, allele, activity, complex partners, stability, half life, and the like) of one or more biomarker of interest. Biosignatures can be used to evaluate diagnostic and/or prognostic criteria such as presence of disease, disease staging, disease monitoring, disease stratification, or surveillance for detection, metastasis or recurrence or progression of disease. For example, the presence or level of microvesicle surface antigen may be used to generate a biosignature for a selected condition or disease. A biosignature can also be used clinically in making decisions concerning treatment modalities including therapeutic intervention. A biosignature can further be used clinically to make treatment decisions, including whether to perform surgery or what treatment standards should be used along with surgery (e.g., either pre-surgery or post-surgery). As an illustrative example, a biosignature of circulating biomarkers that indicates an aggressive form of cancer may call for a more aggressive surgical procedure and/or more aggressive therapeutic regimen to treat the patient. Such therapy related diagnostics are commonly referred to as theranostics.

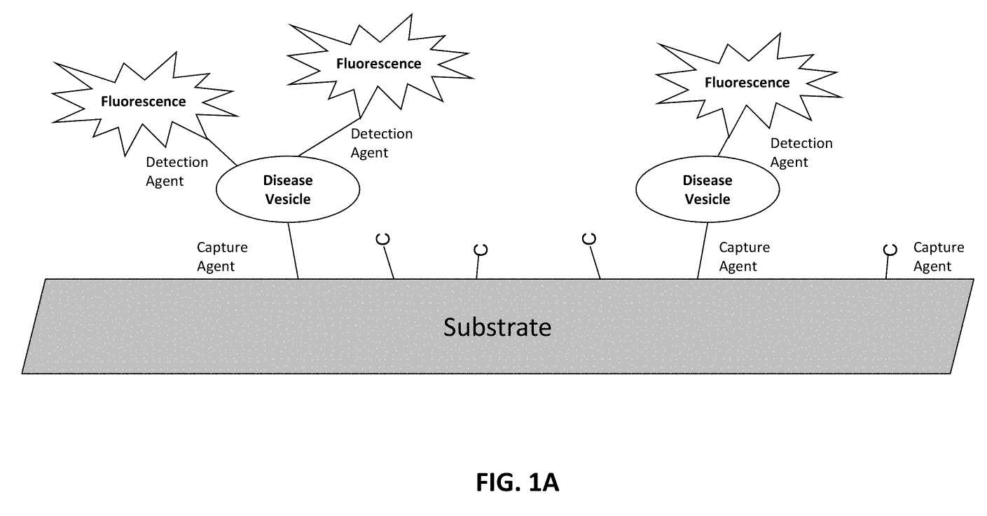

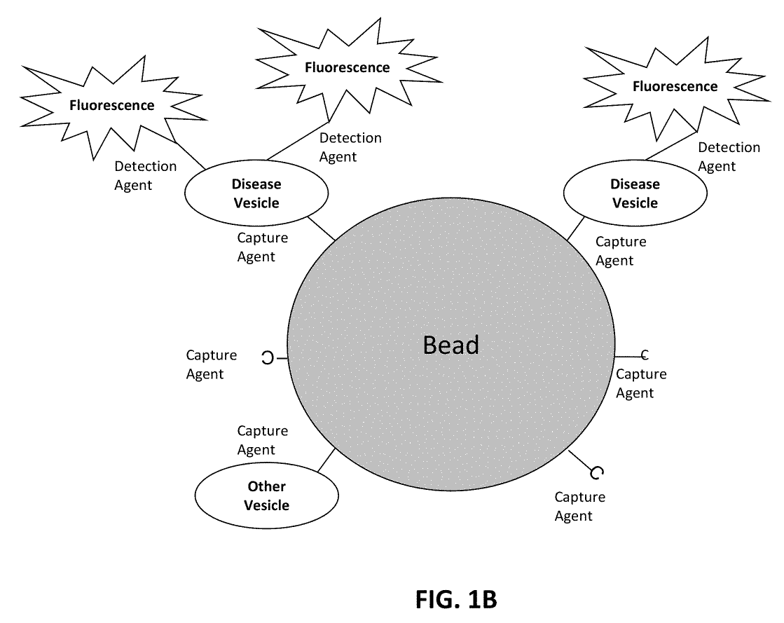

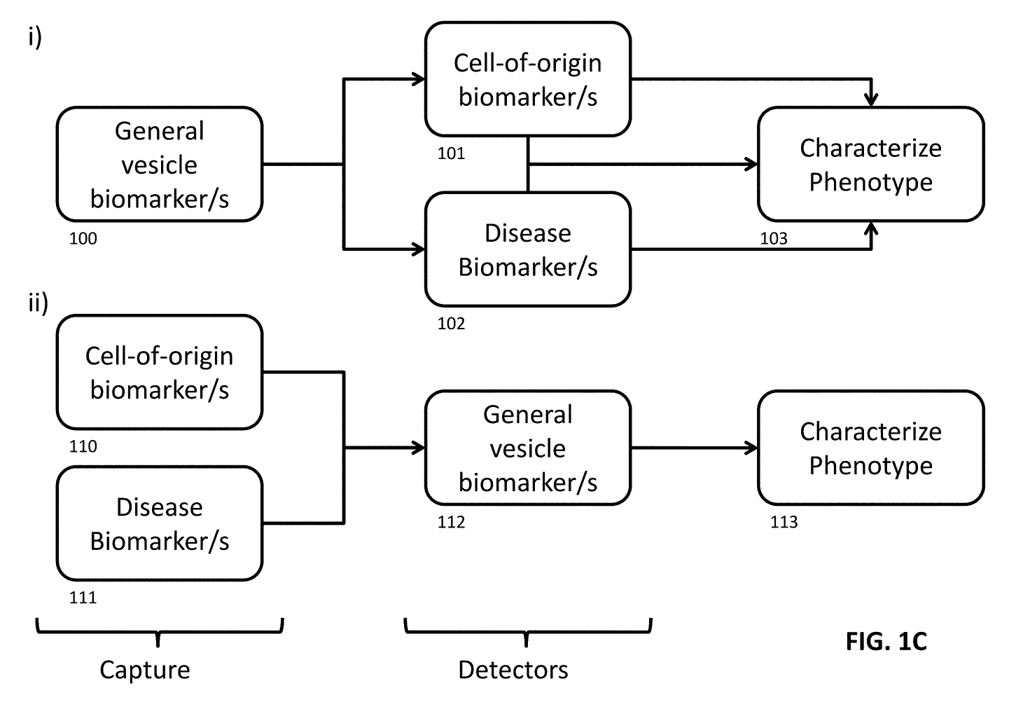

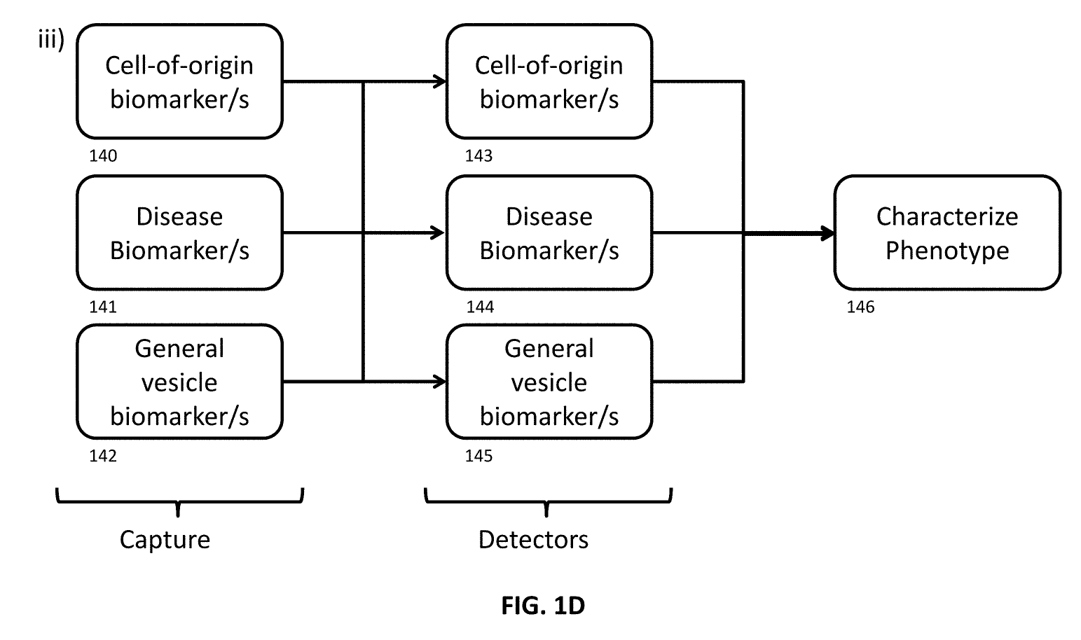

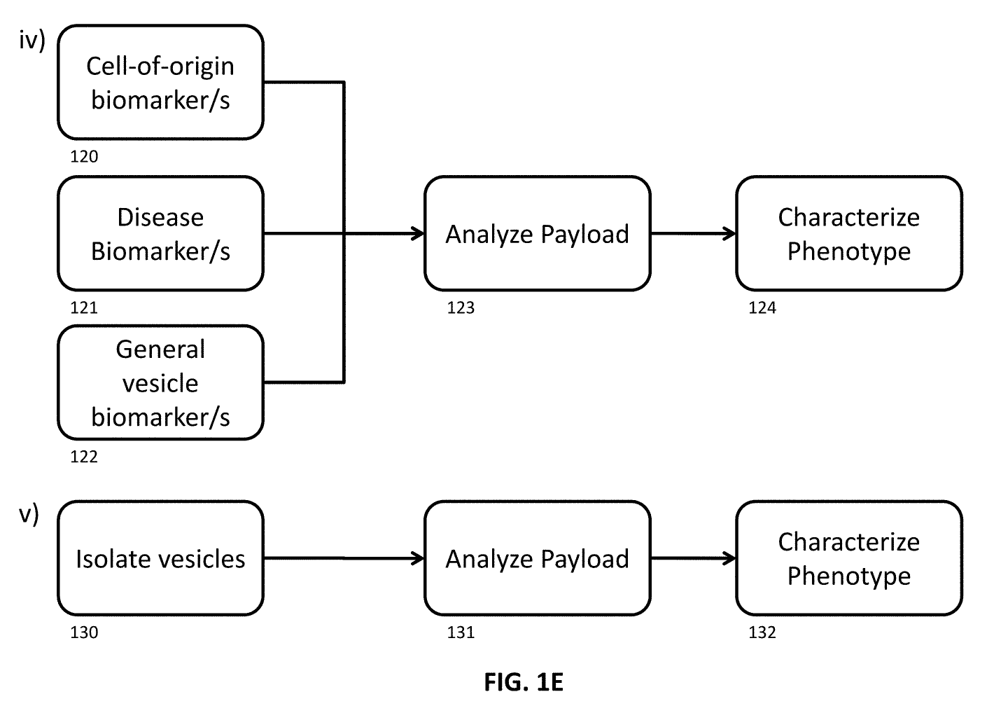

A biosignature can be used in any methods disclosed herein, e.g., to assess whether a subject is afflicted with disease, is at risk for developing disease or to assess the stage or progression of the disease. For example, a biosignature can be used to assess whether a subject has prostate cancer, colon cancer, or other cancer as described herein. Furthermore, a biosignature can be used to determine a stage of a disease or condition, such as colon cancer. The biosignature/biomarker profile comprising a microvesicle can include assessment of payload within the microvesicle. For example, the methods provided herein can be used to isolate a microvesicle population, thereby providing a biosignature of microvesicle antigens. As desired, the payload content within the captured microvesicles can be assessed, thereby providing further biomarker readout of the payload content. See, e.g., FIGS. 1A-1E and related discussion herein.

A biosignature for characterizing a phenotype may comprise any number of useful criteria. As described further below, the term “phenotype” as used herein can mean any trait or characteristic that is attributed to a biosignature/biomarker profile. A phenotype can be detected or identified in part or in whole using the compositions and/or methods of the invention. In some embodiments, at least one criterion is used for each biomarker. In some embodiments, at least 1, 2, 3, 4, 5, 6, 7, 8, 9, 10, 15, 20, 30, 40, 50, 60, 70, 80, 90 or at least 100 criteria are used. For example, for the characterizing of a cancer, a number of different criteria can be used when the subject is diagnosed with a cancer: 1) if the amount of microvesicle associated biomarkers in a sample from a subject is higher than a reference value; 2) if the amount of a biomarker within cell type specific vesicles (i.e. vesicles derived from a specific tissue or organ) is higher than a reference value; or 3) if the amount of a biomarker within vesicles with one or more cancer specific biomarkers is higher than a reference value. Similar rules can apply if the amount of microvesicles is less than or the same as the reference. The method can further include a quality control measure, such that the results are provided for the subject if the samples meet the quality control measure. In some embodiments, if the criteria are met but the quality control is questionable, the subject is reassessed.

### Biomarker Detection

The compositions and methods of the invention can be used to assess any useful biomarkers in a biological sample for charactering a phenotype associated with the sample. Such biomarkers include all sorts of biological entities such as proteins, nucleic acids, lipids, carbohydrates, complexes of any thereof, and microvesicles. Various molecules associated with a microvesicle surface or enclosed within the microvesicle lumen (referred to herein as “payload”) can serve as biomarkers. The microvesicles themselves can also be used as biomarkers.

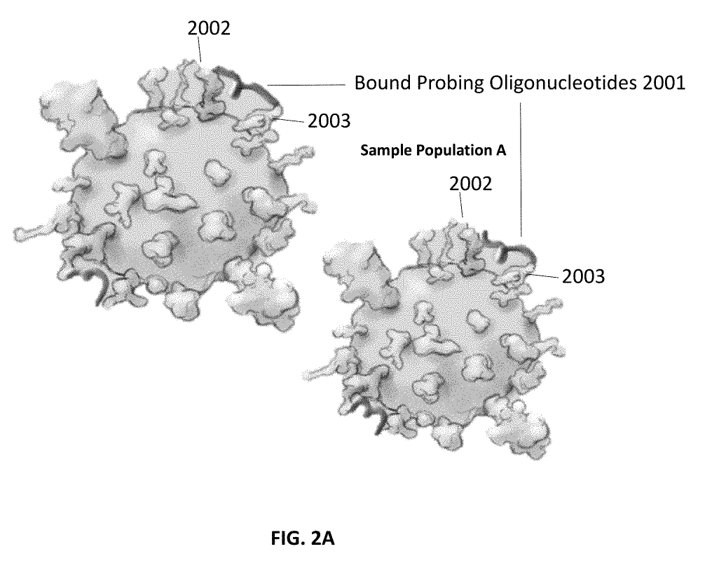

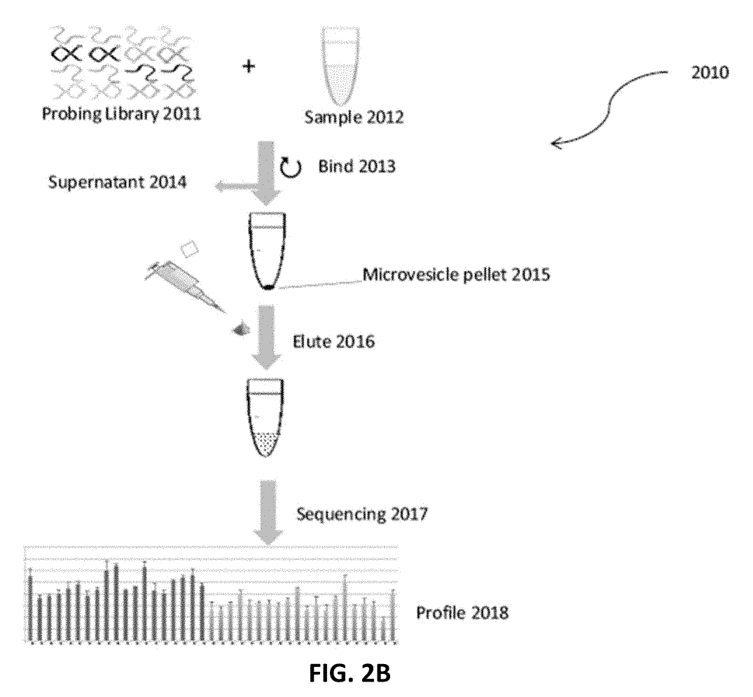

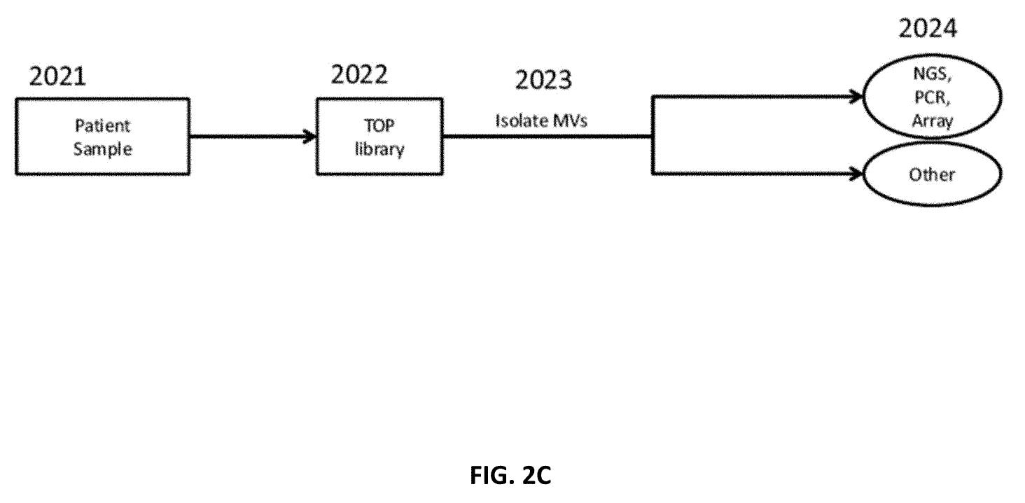

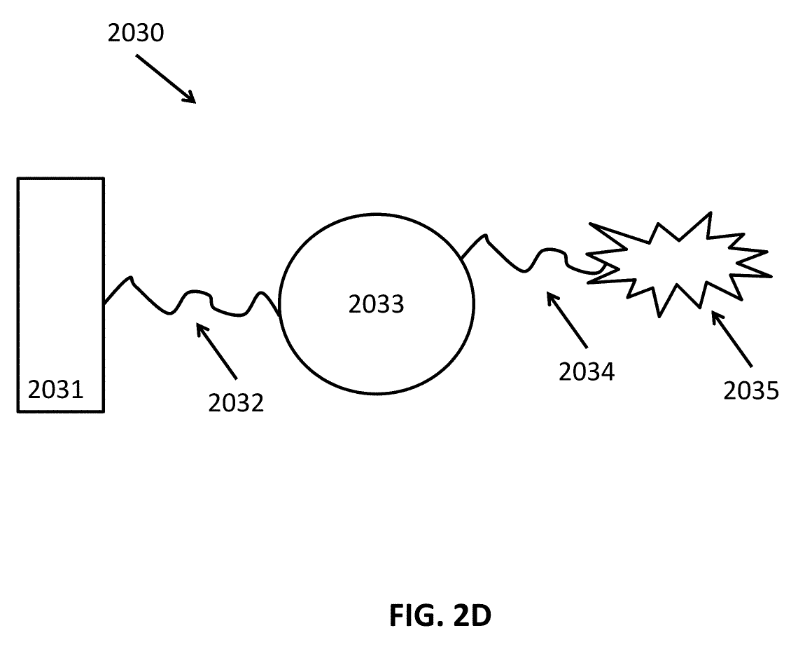

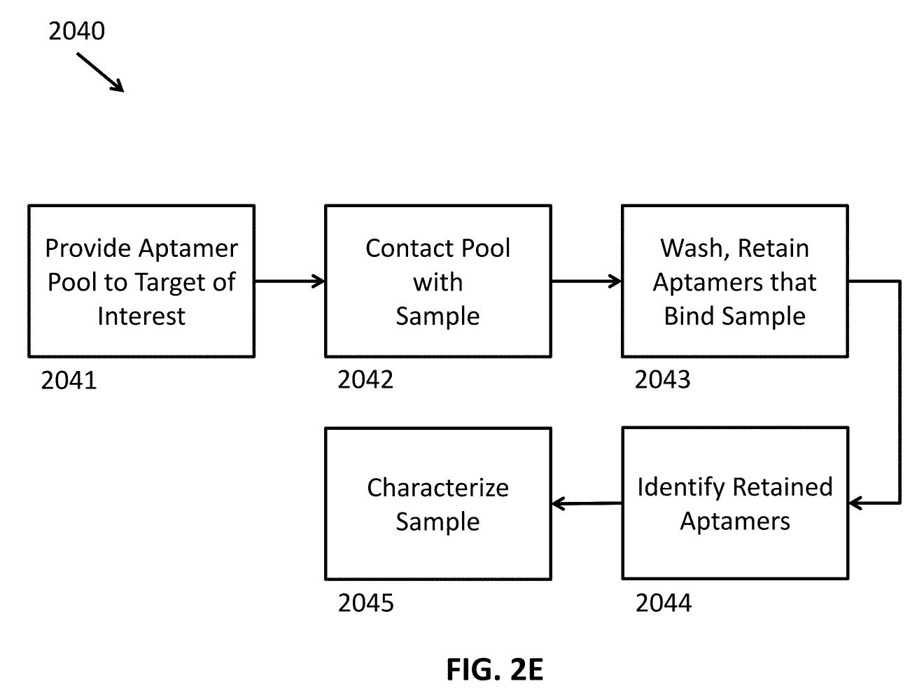

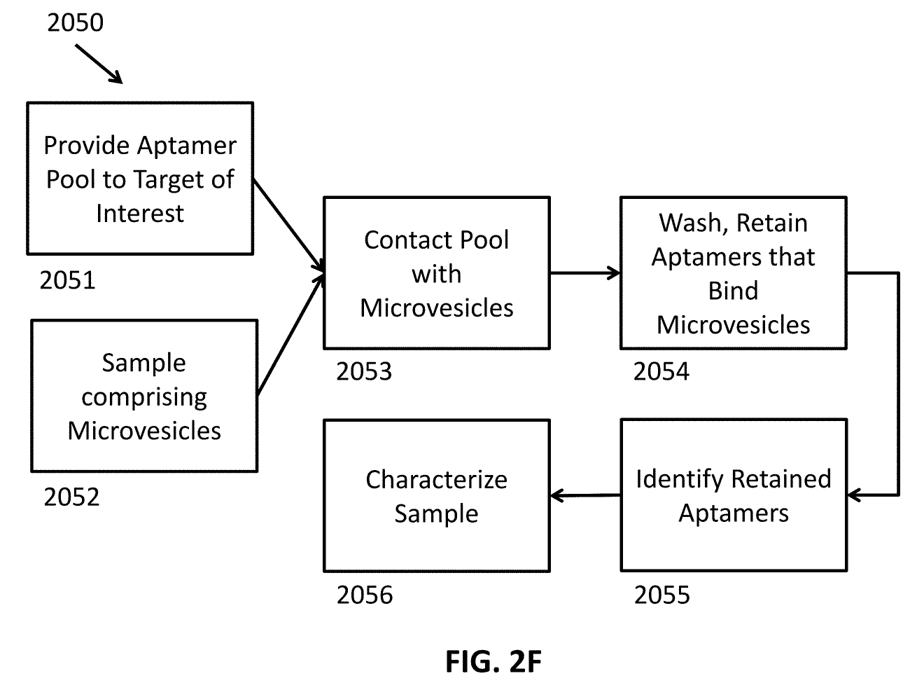

The methods of the invention can be used to assess levels or presence of a microvesicle population. In some embodiments, aptamer libraries are used as binding agents to characterize the microvesicles. See, e.g., FIGS. 2E-F. Such aptamer libraries can also be used to assess levels or presence of their specific target molecule. See, e.g., FIG. 2D. Binding agents such as antibodies or aptamers can be used to capture or isolated a component present in a biological sample that has the agent's target molecule present. For example, if a given microvesicle surface antigen is present on a cell, cell fragment or cell-derived extracellular vesicle. A binding agent to the biomarker, including without limitation an aptamer or plurality of aptamers, may be used to capture or isolate the cell, cell fragment or cell-derived extracellular vesicles. See, e.g., FIGS. 1A-B, 2D. Such captured or isolated entities may be further characterized to assess additional surface antigens or internal “payload” molecules present (i.e., nucleic acid molecules, lipids, sugars, polypeptides or functional fragments thereof, or anything else present in the cellular milieu that may be used as a biomarker), where one or more biomarkers provide a biosignature to assess a desired phenotype, such as disease or condition. See, e.g., FIG. 1E. Therefore, the methods provided herein are used not only to assess one or more microvesicle surface antigen of interest but are also used to separate a component present in a biological sample, where the components themselves can be further assessed to identify a biosignature.

The methods of the invention can comprise multiplex analysis of multiple biomarkers, e.g., at least 2, 3, 4, 5, 6, 7, 8, 9, 10, 11, 12, 13, 14, 15, 16, 17, 18, 19, 20, 25, 50, 75 or 100 different biomarkers. For example, an assay of a heterogeneous population of vesicles can be performed with a plurality of particles that are differentially labeled. There can be at least 2, 3, 4, 5, 6, 7, 8, 9, 10, 11, 12, 13, 14, 15, 16, 17, 18, 19, 20, 25, 50, 75 or 100 differentially labeled particles. The particles may be externally labeled, such as with a tag, or they may be intrinsically labeled. Each differentially labeled particle can be coupled to a capture agent, such as a binding agent, for a vesicle, resulting in capture of a vesicle. The multiple capture agents can be selected to characterize a phenotype of interest, including capture agents against general vesicle biomarkers, cell-of-origin specific biomarkers, and disease biomarkers. One or more biomarkers of the captured vesicle can then be detected by a plurality of binding agents. The binding agent can be directly labeled to facilitate detection. Alternatively, the binding agent is labeled by a secondary agent. For example, the binding agent may be an antibody for a biomarker on the vesicle, wherein the binding agent is linked to biotin. A secondary agent comprises streptavidin linked to a reporter and can be added to detect the biomarker. In some embodiments, the captured vesicle is assayed for at least 2, 3, 4, 5, 6, 7, 8, 9, 10, 11, 12, 13, 14, 15, 16, 17, 18, 19, 20, 25, 50, 75 or 100 different biomarkers. For example, multiple detectors, i.e., detection of multiple biomarkers of a captured vesicle or population of vesicles, can increase the signal obtained, permitted increased sensitivity, specificity, or both, and the use of smaller amounts of samples. Detection can be with more than one biomarker, including without limitation more than one vesicle marker such as in any of Tables 3 or 6.

An immunoassay based method (e.g., sandwich assay) can be used to detect a biomarker of a vesicle. An example includes ELISA. A binding agent can be bound to a well. For example, a binding agent such as an aptamer or antibody to an antigen of a vesicle can be attached to a well. A biomarker on the captured vesicle can be detected based on the methods described herein. FIG. 1A shows an illustrative schematic for a sandwich-type of immunoassay. The capture agent can be against a vesicle antigen of interest, e.g., a general vesicle biomarker, a cell-of-origin marker, or a disease marker. In the figure, the captured vesicles are detected using fluorescently labeled binding agent (detection agent) against vesicle antigens of interest. Multiple capture binding agents can be used, e.g., in distinguishable addresses on an array or different wells of an immunoassay plate. The detection binding agents can be against the same antigen as the capture binding agent, or can be directed against other markers. The capture binding agent can be any useful binding agent, e.g., tethered aptamers, antibodies or lectins, and/or the detector antibodies can be similarly substituted, e.g., with detectable (e.g., labeled) aptamers, antibodies, lectins or other binding proteins or entities. In some embodiments, one or more capture agents to a general vesicle biomarker, a cell-of-origin marker, and/or a disease marker are used along with detection agents against general vesicle biomarker, such as tetraspanin molecules including without limitation one or more of CD9, CD63 and CD81, or other markers in Table 3 herein. Examples of microvesicle surface antigens are disclosed herein, e.g. in Tables 3 or 6, or are known in the art, and examples useful in methods and compositions of the invention are disclosed of International Patent Application Nos. PCT/US2009/62880, filed Oct. 30, 2009; PCT/US2009/006095, filed Nov. 12, 2009; PCT/US2011/26750, filed Mar. 1, 2011; PCT/US2011/031479, filed Apr. 6, 2011; PCT/US11/48327, filed Aug. 18, 2011; PCT/US2008/71235, filed Jul. 25, 2008; PCT/US10/58461, filed Nov. 30, 2010; PCT/US2011/21160, filed Jan. 13, 2011; PCT/US2013/030302, filed Mar. 11, 2013; PCT/US12/25741, filed Feb. 17, 2012; PCT/2008/76109, filed Sep. 12, 2008; PCT/US12/42519, filed Jun. 14, 2012; PCT/US12/50030, filed Aug. 8, 2012; PCT/US12/49615, filed Aug. 3, 2012; PCT/US12/41387, filed Jun. 7, 2012; PCT/US2013/072019, filed Nov. 26, 2013; PCT/US2014/039858, filed May 28, 2013; PCT/IB2013/003092, filed Oct. 23, 2013; PCT/US13/76611, filed Dec. 19, 2013; PCT/US14/53306, filed Aug. 28, 2014; and PCT/US15/62184, filed Nov. 23, 2015; each of which applications is incorporated herein by reference in its entirety.

FIG. 1C presents an illustrative schematic for analyzing vesicles according to the methods of the invention. Capture agents are used to capture vesicles, detectors are used to detect the captured vesicles, and the level or presence of the captured and detected microvesicles is used to characterize a phenotype. Capture agents, detectors and characterizing phenotypes can be any of those described herein. For example, capture agents include antibodies or aptamers tethered to a substrate that recognize a vesicle antigen of interest, detectors include labeled antibodies or aptamers to a vesicle antigen of interest, and characterizing a phenotype includes a diagnosis, prognosis, or theranosis of a disease. In the scheme shown in FIG. 1C i), a population of vesicles is captured with one or more capture agents against general vesicle biomarkers (100). The captured vesicles are then labeled with detectors against cell-of-origin biomarkers (101) and/or disease specific biomarkers (102). If only cell-of-origin detectors are used (101), the biosignature used to characterize the phenotype (103) can include the general vesicle markers (100) and the cell-of-origin biomarkers (101). If only disease detectors are used (102), the biosignature used to characterize the phenotype (103) can include the general vesicle markers (100) and the disease biomarkers (102). Alternately, detectors are used to detect both cell-of-origin biomarkers (101) and disease specific biomarkers (102). In this case, the biosignature used to characterize the phenotype (103) can include the general vesicle markers (100), the cell-of-origin biomarkers (101) and the disease biomarkers (102). The biomarkers combinations are selected to characterize the phenotype of interest. Non-limiting examples of biomarkers and phenotypes are described herein, e.g., in Tables 1, 3 or 6.

In the scheme shown in FIG. 1C ii), a population of vesicles is captured with one or more capture agents against cell-of-origin biomarkers (110) and/or disease biomarkers (111). The captured vesicles are then detected using detectors against general vesicle biomarkers (112). If only cell-of-origin capture agents are used (110), the biosignature used to characterize the phenotype (113) can include the cell-of-origin biomarkers (110) and the general vesicle markers (112). If only disease biomarker capture agents are used (111), the biosignature used to characterize the phenotype (113) can include the disease biomarkers (111) and the general vesicle biomarkers (112). Alternately, capture agents to one or more cell-of-origin biomarkers (110) and one or more disease specific biomarkers (111) are used to capture vesicles. In this case, the biosignature used to characterize the phenotype (113) can include the cell-of-origin biomarkers (110), the disease biomarkers (111), and the general vesicle markers (113). The biomarkers combinations are selected to characterize the phenotype of interest and can be selected from the biomarkers and phenotypes described herein.

The methods of the invention comprise capture and detection of microvesicles of interest using any combination of useful biomarkers. For example, a microvesicle population can be captured using one or more binding agent to any desired combination of cell of origin, disease specific, or general vesicle markers. The captured microvesicles can then be detected using one or more binding agent to any desired combination of cell of origin, disease specific, or general vesicle markers. FIG. 1D represents a flow diagram of such configurations. Any one or more of a cell-of-origin biomarker (140), disease biomarkers (141), and general vesicle biomarker (142) is used to capture a microvesicle population. Thereafter, any one or more of a cell-of-origin biomarker (143), disease biomarkers (144), and general vesicle biomarker (145) is used to detect the captured microvesicle population. The biosignature of captured and detected microvesicles is then used to characterize a phenotype. The biomarkers combinations are selected to characterize the phenotype of interest and can be selected from the biomarkers and phenotypes described herein.

A microvesicle payload molecule can be assessed as a member of a biosignature panel. A payload molecule comprises any of the biological entities contained within a cell, cell fragment or vesicle membrane. These entities include without limitation nucleic acids, e.g., mRNA, microRNA, or DNA fragments; protein, e.g., soluble and membrane associated proteins; carbohydrates; lipids; metabolites; and various small molecules, e.g., hormones. The payload can be part of the cellular milieu that is encapsulated as a vesicle is formed in the cellular environment. In some embodiments of the invention, the payload is analyzed in addition to detecting vesicle surface antigens. Specific populations of vesicles can be captured as described above then the payload in the captured vesicles can be used to characterize a phenotype. For example, vesicles captured on a substrate can be further isolated to assess the payload therein. Alternately, the vesicles in a sample are detected and sorted without capture. The vesicles so detected can be further isolated to assess the payload therein. In some embodiments, vesicle populations are sorted by flow cytometry and the payload in the sorted vesicles is analyzed. In the scheme shown in FIG. 1E iv), a population of vesicles is captured and/or detected (120) using one or more of cell-of-origin biomarkers (120), disease biomarkers (121), and/or general vesicle markers (122). The payload of the isolated vesicles is assessed (123). A biosignature detected within the payload can be used to characterize a phenotype (124). In a non-limiting example, a vesicle population can be analyzed in a plasma sample from a patient using antibodies against one or more vesicle antigens of interest. The antibodies can be capture antibodies which are tethered to a substrate to isolate a desired vesicle population. Alternately, the antibodies can be directly labeled and the labeled vesicles isolated by sorting with flow cytometry. The presence or level of microRNA or mRNA extracted from the isolated vesicle population can be used to detect a biosignature. The biosignature is then used to diagnose, prognose or theranose the patient.

In other embodiments, vesicle or cellular payload is analyzed in a population (e.g., cells or vesicles) without first capturing or detected subpopulations of vesicles. For example, a cellular or extracellular vesicle population can be generally isolated from a sample using centrifugation, filtration, chromatography, or other techniques as described herein and known in the art. The payload of such sample components can be analyzed thereafter to detect a biosignature and characterize a phenotype. In the scheme shown in FIG. 1E v), a population of vesicles is isolated (130) and the payload of the isolated vesicles is assessed (131). A biosignature detected within the payload can be used to characterize a phenotype (132). In a non-limiting example, a vesicle population is isolated from a plasma sample from a patient using size exclusion and membrane filtration. The presence or level of microRNA or mRNA extracted from the vesicle population is used to detect a biosignature. The biosignature is then used to diagnose, prognose or theranose the patient.

The biomarkers used to detect a vesicle population can be selected to detect a microvesicle population of interest, e.g., a population of vesicles that provides a diagnosis, prognosis or theranosis of a selected condition or disease, including but not limited to a cancer, a premalignant condition, an inflammatory disease, an immune disease, an autoimmune disease or disorder, a cardiovascular disease or disorder, neurological disease or disorder, infectious disease or pain. See Section “Phenotypes” herein for more detail. In some embodiments, the biomarkers are selected from the group consisting of EpCam (epithelial cell adhesion molecule), CD9 (tetraspanin CD9 molecule), PCSA (prostate cell specific antigen, see Rokhlin et al., 5E10: a prostate-specific surface-reactive monoclonal antibody. Cancer Lett. 1998 131:129-36), CD63 (tetraspanin CD63 molecule), CD81 (tetraspanin CD81 molecule), PSMA (FOLH1, folate hydrolase (prostate-specific membrane antigen) 1), B7H3 (CD276 molecule), PSCA (prostate stem cell antigen), ICAM (intercellular adhesion molecule), STEAP (STEAP1, six transmembrane epithelial antigen of the prostate 1), KLK2 (kallikrein-related peptidase 2), SSX2 (synovial sarcoma, X breakpoint 2), SSX4 (synovial sarcoma, X breakpoint 4), PBP (prostatic binding protein), SPDEF (SAM pointed domain containing ets transcription factor), EGFR (epidermal growth factor receptor), and a combination thereof. One or more of these markers can provide a biosignature for a specific condition, such as to detect a cancer, including without limitation a carcinoma, a prostate cancer, a breast cancer, a lung cancer, a colorectal cancer, an ovarian cancer, melanoma, a brain cancer, or other type of cancer as disclosed herein. In some embodiments, a binding agent to one or more of these markers is used to capture a microvesicle population, and another binding agent is used to assist in detection of the capture vesicles as described herein. The binding agents can be any useful binding agent as disclosed herein or known in the art, e.g., antibodies or aptamers.

The methods of characterizing a phenotype can employ a combination of techniques to assess a component or population of components present in a biological sample of interest. For example, an antibody or aptamer can be used to assess a single cell, or a single extracellular vesicle or a population of cells or population of vesicles. A sample may be split into various aliquots, where each is analyzed separately. For example, protein content of one or more aliquot is determined and microRNA content of one or more other aliquot is determined. The protein content and microRNA content can be combined to characterize a phenotype. In some embodiments, a component present in a biological sample of interest is isolated and the payload therein is assessed (e.g., capture a population of subpopulation of vesicles using a binding agent and further assess nucleic acid or proteins present in the isolated vesicles).

In some embodiments, a population of vesicles with a given surface marker can be isolated by using a binding agent to a microvesicle surface marker. See, e.g., FIGS. 1A, 1B. The binding agent can be an aptamer that was identified to target the microvesicle surface marker using to the methods of the invention. The isolated vesicles is assessed for additional biomarkers such as surface content or payload, which can be contemporaneous to detection of the aptamer-specific target or the assessment of additional biomarkers can be before or subsequent to aptamer-specific target detection.

A biosignature can be detected qualitatively or quantitatively by detecting a presence, level or concentration of a circulating biomarker, e.g., a microRNA, protein, vesicle or other biomarker, as disclosed herein. These biosignature components can be detected using a number of techniques known to those of skill in the art. For example, a biomarker can be detected by microarray analysis, polymerase chain reaction (PCR) (including PCR-based methods such as real time polymerase chain reaction (RT-PCR), quantitative real time polymerase chain reaction (Q-PCR/qPCR) and the like), hybridization with allele-specific probes, enzymatic mutation detection, ligation chain reaction (LCR), oligonucleotide ligation assay (OLA), flow-cytometric heteroduplex analysis, chemical cleavage of mismatches, mass spectrometry, nucleic acid sequencing, single strand conformation polymorphism (SSCP), denaturing gradient gel electrophoresis (DGGE), temperature gradient gel electrophoresis (TGGE), restriction fragment polymorphisms, serial analysis of gene expression (SAGE), or combinations thereof. A biomarker, such as a nucleic acid, can be amplified prior to detection. A biomarker can also be detected by immunoassay, immunoblot, immunoprecipitation, enzyme-linked immunosorbent assay (ELISA; EIA), radioimmunoassay (RIA), flow cytometry, or electron microscopy (EM).

Biosignatures can be detected using binding agent that function as either as capture agents and detection agents, as described herein. A capture agent can comprise an antibody, aptamer or other entity which recognizes a biomarker and can be used for capturing the biomarker. Biomarkers that can be captured include circulating biomarkers, e.g., a protein, nucleic acid, lipid or biological complex in solution in a bodily fluid. Similarly, the capture agent can be used for capturing a vesicle. A detection agent can comprise an antibody or other entity which recognizes a biomarker and can be used for detecting the biomarker vesicle, or which recognizes a vesicle and is useful for detecting a vesicle. In some embodiments, the detection agent is labeled and the label is detected, thereby detecting the biomarker or vesicle. The detection agent can be a binding agent, e.g., an antibody or aptamer. In other embodiments, the detection agent comprises a small molecule such as a membrane protein labeling agent. See, e.g., the membrane protein labeling agents disclosed in Alroy et al., US. Patent Publication US 2005/0158708. In some embodiments, vesicles are isolated or captured as described herein, and one or more membrane protein labeling agent is used to detect the vesicles. In many cases, the antigen or other vesicle-moiety that is recognized by the capture and detection agents are interchangeable.

In a non-limiting embodiment, a vesicle having a cell-of-origin specific antigen on its surface and a cancer-specific antigen on its surface, is captured using a binding agent that is specific to a cells-specific antigen, e.g., by tethering the capture antibody or aptamer to a substrate, and then the vesicle is detected using a binding agent to a disease-specific antigen, e.g., by labeling the binding agent used for detection with a fluorescent dye and detecting the fluorescent radiation emitted by the dye.

It will be apparent to one of skill in the art that where the target molecule for a binding agent is informative as to assessing a condition or disease, the same binding agent can be used to both capture a component comprising the target molecule (e.g., microvesicle surface antigen of interest) and also be modified to comprise a detectable label so as to detect the target molecule, e.g., binding agent₁-antigen-binding agent₂*, wherein the * signifies a detectable label; binding agent₁ and binding agent₂ may be the same binding agent or a different binding agent (e.g., same aptamer or different aptamer). In addition, binding agent₁ and binding agent₂ can be selected from wholly different categories of binding agents (e.g., antibody, aptamer, synthetic antibody, peptide-nucleic acid molecule, or any molecule that is configured to specifically bind to or associate with its target molecule). Such binding molecules can be selected solely based on their binding specificity for a target molecule. Examples of additional biomarkers that can be incorporated into the methods and compositions of the invention are known in the art, such as those disclosed in International Patent Publication Nos. WO/2012/174282 (Int'l Appl. PCT/US2012/042519 filed Jun. 14, 2012) and WO/2013/020995 (Int'l Appl. PCT/US2012/050030 filed Aug. 8, 2013). The detectable signal can itself be associated with a nucleic acid molecule that hybridizes with a stretch of nucleic acids present in each oligonucleotide comprising a probing library. The stretch can be the same or different as to one or more oligonucleotides in a library. The detectable signal can comprise fluorescence agents, including color-coded barcodes which are known, such as in U.S. Patent Application Pub. No. 20140371088, 2013017837, and 20120258870.

Techniques of detecting biomarkers or capturing sample components using various binding agents include the use of a planar substrate such as an array (e.g., biochip or microarray), with molecules immobilized to the substrate as capture agents that facilitate the detection of a particular biosignature. The array can be provided as part of a kit for assaying one or more biomarkers. Additional examples of binding agents described above and useful in the compositions and methods of the invention are disclosed in International Patent Publication No. WO/2011/127219, entitled “Circulating Biomarkers for Disease” and filed Apr. 6, 2011, which application is incorporated by reference in its entirety herein. Vesicles isolated by the methods herein can be assessed by array for detection and diagnosis of diseases including presymptomatic diseases. In some embodiments, an array comprises a custom array comprising biomolecules selected to specifically identify biomarkers of interest. Customized arrays can be modified to detect biomarkers that increase statistical performance, e.g., additional biomolecules that identifies a biosignature which lead to improved cross-validated error rates in multivariate prediction models (e.g., logistic regression, discriminant analysis, or regression tree models). In some embodiments, customized array(s) are constructed to study the biology of a disease, condition or syndrome and profile biosignatures in defined physiological states. Markers for inclusion on the customized array be chosen based upon statistical criteria, e.g., having a desired level of statistical significance in differentiating between phenotypes or physiological states. In some embodiments, standard significance of p-value=0.05 is chosen to exclude or include biomolecules on the microarray. The p-values can be corrected for multiple comparisons. As an illustrative example, nucleic acids extracted from samples from a subject with or without a disease can be hybridized to a high density microarray that binds to thousands of gene sequences. Nucleic acids whose levels are significantly different between the samples with or without the disease can be selected as biomarkers to distinguish samples as having the disease or not. A customized array can be constructed to detect the selected biomarkers. In some embodiments, customized arrays comprise low density microarrays, which refer to arrays with lower number of addressable binding agents, e.g., tens or hundreds instead of thousands. Low density arrays can be formed on a substrate. In some embodiments, customizable low density arrays use PCR amplification in plate wells, e.g., TaqMan® Gene Expression Assays (Applied Biosystems by Life Technologies Corporation, Carlsbad, Calif.).

A useful binding agent may be linked directly or indirectly to a solid surface or substrate. See, e.g., FIGS. 1A-1B, 2D. A solid surface or substrate can be any physically separable solid to which a binding agent can be directly or indirectly attached including, but not limited to, surfaces provided by microarrays and wells, particles such as beads, columns, optical fibers, wipes, glass and modified or functionalized glass, quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polypropylene, polyethylene, polybutylene, polyurethanes, Teflon material, etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In addition, as is known the art, the substrate may be coated using passive or chemically-derivatized coatings with any number of materials, including polymers, such as dextrans, acrylamides, gelatins or agarose. Such coatings can facilitate the use of the array with a biological sample.

As provided in the examples, below, an aptamer or other useful binding agent can be conjugated to a detectable entity or label.

Appropriate labels include without limitation a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles, fluorophores, quantum dots, or radioactive labels. Protein labels include green fluorescent protein (GFP) and variants thereof (e.g., cyan fluorescent protein and yellow fluorescent protein); and luminescent proteins such as luciferase, as described below. Radioactive labels include without limitation radioisotopes (radionuclides), such as ³H, ¹¹C, ¹⁴C, ¹⁸F, ³²F, ³²P, ³⁵S, ⁶⁴Cu, ⁶⁸Ga, ⁸⁶Y, ⁹⁹Te, ¹¹¹In, ¹²³I, ¹²⁴I, ¹²⁵I, ¹³¹I, ¹³³Xe, ¹⁷⁷Lu, ²¹¹At, or ²¹³Bi. Fluorescent labels include without limitation a rare earth chelate (e.g., europium chelate), rhodamine; fluorescein types including without limitation FITC, 5-carboxyfluorescein, 6-carboxy fluorescein; a rhodamine type including without limitation TAMRA; dansyl; Lissamine; cyanines; phycoerythrins; Texas Red; Cy3, Cy5, dapoxyl, NBD, Cascade Yellow, dansyl, PyMPO, pyrene, 7-diethylaminocoumarin-3-carboxylic acid and other coumarin derivatives, Marina Blue™, Pacific Blue™, Cascade Blue™, 2-anthracenesulfonyl, PyMPO, 3,4,9,10-perylene-tetracarboxylic acid, 2,7-difluorofluorescein (Oregon Green™ 488-X), 5-carboxyfluorescein, Texas Red™-X, Alexa Fluor 430, 5-carboxytetramethylrhodamine (5-TAMRA), 6-carboxytetramethylrhodamine (6-TAMRA), BODIPY FL, bimane, and Alexa Fluor 350, 405, 488, 500, 514, 532, 546, 555, 568, 594, 610, 633, 647, 660, 680, 700, and 750, and derivatives thereof, among many others. See, e.g., “The Handbook—A Guide to Fluorescent Probes and Labeling Technologies,” Tenth Edition, available on the internet at probes (dot) invitrogen (dot) com/handbook. The fluorescent label can be one or more of FAM, dRHO, 5-FAM, 6FAM, dR6G, JOE, HEX, VIC, TET, dTAMRA, TAMRA, NED, dROX, PET, BHQ, Gold540 and LIZ.

Using conventional techniques, an aptamer can be directly or indirectly labeled, e.g., the label is attached to the aptamer through biotin-streptavidin (e.g., synthesize a biotinylated aptamer, which is then capable of binding a streptavidin molecule that is itself conjugated to a detectable label; non-limiting example is streptavidin, phycoerythrin conjugated (SAPE)). Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Alternatively, an aptamer is not labeled, but is later contacted with a second antibody that is labeled after the first antibody is bound to an antigen of interest.

Various enzyme-substrate labels may also be used in conjunction with a composition or method of the invention. Such enzyme-substrate labels are available commercially (e.g., U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of enzyme-substrate combinations include, but are not limited to, horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-β-D-galactosidase.

Aptamer(s) can be linked to a substrate such as a planar substrate. A planar array generally contains addressable locations (e.g., pads, addresses, or micro-locations) of biomolecules in an array format. The size of the array will depend on the composition and end use of the array. Arrays can be made containing from 2 different molecules to many thousands. Generally, the array comprises from two to as many as 100,000 or more molecules, depending on the end use of the array and the method of manufacture. A microarray for use with the invention comprises at least one biomolecule that identifies or captures a biomarker present in a biosignature of interest, e.g., a microRNA or other biomolecule or vesicle that makes up the biosignature. In some arrays, multiple substrates are used, either of different or identical compositions. Accordingly, planar arrays may comprise a plurality of smaller substrates.

The present invention can make use of many types of arrays for detecting a biomarker, e.g., a biomarker associated with a biosignature of interest. Useful arrays or microarrays include without limitation DNA microarrays, such as cDNA microarrays, oligonucleotide microarrays and SNP microarrays, microRNA arrays, protein microarrays, antibody microarrays, tissue microarrays, cellular microarrays (also called transfection microarrays), chemical compound microarrays, and carbohydrate arrays (glycoarrays). These arrays are described in more detail above. In some embodiments, microarrays comprise biochips that provide high-density immobilized arrays of recognition molecules (e.g., aptamers or antibodies), where biomarker binding is monitored indirectly (e.g., via fluorescence).

An array or microarray that can be used to detect one or more biomarkers of a biosignature and comprising one or more aptamers of the invention can be made according to the methods described in U.S. Pat. Nos. 6,329,209; 6,365,418; 6,406,921; 6,475,808; and 6,475,809, and U.S. patent application Ser. No. 10/884,269, each of which is herein incorporated by reference in its entirety. Custom arrays to detect specific selections of sets of biomarkers described herein can be made using the methods described in these patents. Commercially available microarrays can also be used to carry out the methods of the invention, including without limitation those from Affymetrix (Santa Clara, Calif.), Illumina (San Diego, Calif.), Agilent (Santa Clara, Calif.), Exiqon (Denmark), or Invitrogen (Carlsbad, Calif.). Custom and/or commercial arrays include arrays for detection proteins, nucleic acids, and other biological molecules and entities (e.g., cells, vesicles, virii) as described herein.

In some embodiments, multiple capture molecules are disposed on an array, e.g., proteins, peptides or additional nucleic acid molecules. In certain embodiments, the proteins are immobilized using methods and materials that minimize the denaturing of the proteins, that minimize alterations in the activity of the proteins, or that minimize interactions between the protein and the surface on which they are immobilized. The capture molecules can comprise one or more antibody or aptamer. In some embodiments, an array is constructed for the hybridization of a pool of aptamers. The array can then be used to identify pool members that bind a sample, thereby facilitating characterization of a phenotype. See FIGS. 2E-F and related disclosure for further details.

Array surfaces useful may be of any desired shape, form, or size. Non-limiting examples of surfaces include chips, continuous surfaces, curved surfaces, flexible surfaces, films, plates, sheets, or tubes. Surfaces can have areas ranging from approximately a square micron to approximately 500 cm². The area, length, and width of surfaces may be varied according to the requirements of the assay to be performed. Considerations may include, for example, ease of handling, limitations of the material(s) of which the surface is formed, requirements of detection systems, requirements of deposition systems (e.g., arrayers), or the like.

In certain embodiments, it is desirable to employ a physical means for separating groups or arrays of binding islands or immobilized biomolecules: such physical separation facilitates exposure of different groups or arrays to different solutions of interest. Therefore, in certain embodiments, arrays are situated within microwell plates having any number of wells. In such embodiments, the bottoms of the wells may serve as surfaces for the formation of arrays, or arrays may be formed on other surfaces and then placed into wells. In certain embodiments, such as where a surface without wells is used, binding islands may be formed or molecules may be immobilized on a surface and a gasket having holes spatially arranged so that they correspond to the islands or biomolecules may be placed on the surface. Such a gasket is preferably liquid tight. A gasket may be placed on a surface at any time during the process of making the array and may be removed if separation of groups or arrays is no longer desired.

In some embodiments, the immobilized molecules can bind to one or more biomarkers or vesicles present in a biological sample contacting the immobilized molecules. In some embodiments, the immobilized molecules modify or are modified by molecules present in the one or more vesicles contacting the immobilized molecules. Contacting the sample typically comprises overlaying the sample upon the array.

Modifications or binding of molecules in solution or immobilized on an array can be detected using detection techniques known in the art. Examples of such techniques include immunological techniques such as competitive binding assays and sandwich assays; fluorescence detection using instruments such as confocal scanners, confocal microscopes, or CCD-based systems and techniques such as fluorescence, fluorescence polarization (FP), fluorescence resonant energy transfer (FRET), total internal reflection fluorescence (TIRF), fluorescence correlation spectroscopy (FCS); colorimetric/spectrometric techniques; surface plasmon resonance, by which changes in mass of materials adsorbed at surfaces are measured; techniques using radioisotopes, including conventional radioisotope binding and scintillation proximity assays (SPA); mass spectroscopy, such as matrix-assisted laser desorption/ionization mass spectroscopy (MALDI) and MALDI-time of flight (TOF) mass spectroscopy; ellipsometry, which is an optical method of measuring thickness of protein films; quartz crystal microbalance (QCM), a very sensitive method for measuring mass of materials adsorbing to surfaces; scanning probe microscopies, such as atomic force microscopy (AFM), scanning force microscopy (SFM) or scanning electron microscopy (SEM); and techniques such as electrochemical, impedance, acoustic, microwave, and IR/Raman detection. See, e.g., Mere L, et al., “Miniaturized FRET assays and microfluidics: key components for ultra-high-throughput screening,” Drug Discovery Today 4(8):363-369 (1999), and references cited therein; Lakowicz J R, Principles of Fluorescence Spectroscopy, 2nd Edition, Plenum Press (1999), or Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, Ni: Humana Press, 2007, each of which is herein incorporated by reference in its entirety.

Microarray technology can be combined with mass spectroscopy (MS) analysis and other tools. Electrospray interface to a mass spectrometer can be integrated with a capillary in a microfluidics device. For example, one commercially available system contains eTag reporters that are fluorescent labels with unique and well-defined electrophoretic mobilities; each label is coupled to biological or chemical probes via cleavable linkages. The distinct mobility address of each eTag reporter allows mixtures of these tags to be rapidly deconvoluted and quantitated by capillary electrophoresis. This system allows concurrent gene expression, protein expression, and protein function analyses from the same sample Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, N.J.: Humana Press, 2007, which is herein incorporated by reference in its entirety.

A biochip can include components for a microfluidic or nanofluidic assay. A microfluidic device can be used for isolating or analyzing biomarkers, such as determining a biosignature. Microfluidic systems allow for the miniaturization and compartmentalization of one or more processes for isolating, capturing or detecting a vesicle, detecting a microRNA, detecting a circulating biomarker, detecting a biosignature, and other processes. The microfluidic devices can use one or more detection reagents in at least one aspect of the system, and such a detection reagent can be used to detect one or more biomarkers. In some embodiments, the device detects a biomarker on an isolated or bound vesicle. Various probes, antibodies, proteins, or other binding agents can be used to detect a biomarker within the microfluidic system. The detection agents may be immobilized in different compartments of the microfluidic device or be entered into a hybridization or detection reaction through various channels of the device.

A vesicle in a microfluidic device can be lysed and its contents detected within the microfluidic device, such as proteins or nucleic acids, e.g., DNA or RNA such as miRNA or mRNA. The nucleic acid may be amplified prior to detection, or directly detected, within the microfluidic device. Thus microfluidic system can also be used for multiplexing detection of various biomarkers. In some embodiments, vesicles are captured within the microfluidic device, the captured vesicles are lysed, and a biosignature of microRNA from the vesicle payload is determined. The biosignature can further comprise the capture agent used to capture the vesicle.

Novel nanofabrication techniques are opening up the possibilities for biosensing applications that rely on fabrication of high-density, precision arrays, e.g., nucleotide-based chips and protein arrays otherwise known as heterogeneous nanoarrays. Nanofluidics allows a further reduction in the quantity of fluid analyte in a microchip to nanoliter levels, and the chips used here are referred to as nanochips. See, e.g., Unger M et al., Biotechniques 1999; 27(5):1008-14, Kartalov E P et al., Biotechniques 2006; 40(1):85-90, each of which are herein incorporated by reference in their entireties. Commercially available nanochips currently provide simple one step assays such as total cholesterol, total protein or glucose assays that can be run by combining sample and reagents, mixing and monitoring of the reaction. Gel-free analytical approaches based on liquid chromatography (LC) and nanoLC separations (Cutillas et al. Proteomics, 2005; 5:101-112 and Cutillas et al., Mol Cell Proteomics 2005; 4:1038-1051, each of which is herein incorporated by reference in its entirety) can be used in combination with the nanochips.

An array suitable for identifying a disease, condition, syndrome or physiological status can be included in a kit. A kit can include, an aptamer or antibody to a biomarker of interest, including as non-limiting examples, one or more reagents useful for preparing molecules for immobilization onto binding islands or areas of an array, reagents useful for detecting binding of a vesicle to immobilized molecules, and instructions for use.

Further provided herein is a rapid detection device that facilitates the detection of a particular biosignature in a biological sample. The device can integrate biological sample preparation with polymerase chain reaction (PCR) on a chip. The device can facilitate the detection of a particular biosignature of a vesicle in a biological sample, and an example is provided as described in Pipper et al., Angewandte Chemie, 47(21), p. 3900-3904 (2008), which is herein incorporated by reference in its entirety. A biosignature can be incorporated using micro-/nano-electrochemical system (MEMS/NEMS) sensors and oral fluid for diagnostic applications as described in Li et al., Adv Dent Res 18(1): 3-5 (2005), which is herein incorporated by reference in its entirety.

Particle Arrays

As an alternative to planar arrays, assays using particles, such as bead based assays are also capable of use to detect vesicles isolated by the methods herein. Binding agents such as antibodies or aptamers can be conjugated with commercially available beads. See, e.g., Srinivas et al. Anal. Chem. 2011 Oct. 21, Aptamer functionalized Microgel Particles for Protein Detection; See also, review article on aptamers as therapeutic and diagnostic agents, Brody and Gold, Rev. Mol. Biotech. 2000, 74:5-13.

Multiparametric assays or other high throughput detection assays using bead coatings with cognate ligands and reporter molecules with specific activities consistent with high sensitivity automation can be used. In a bead based assay system, a binding agent for a biomarker or vesicle, such as a capture agent (e.g. capture antibody), can be immobilized on an addressable microsphere. Each binding agent for each individual binding assay can be coupled to a distinct type of microsphere (i.e., microbead) and the assay reaction takes place on the surface of the microsphere, such as depicted in FIG. 1B. A binding agent for a vesicle can be a capture antibody coupled to a bead. Dyed microspheres with discrete fluorescence intensities are loaded separately with their appropriate binding agent or capture probes. The different bead sets carrying different binding agents can be pooled as desired to generate custom bead arrays. Bead arrays are then incubated with the sample in a single reaction vessel to perform the assay.

A bead substrate can provide a platform for attaching one or more binding agents, e.g., antibodies or aptamer(s). For multiplexing, multiple different bead sets (e.g., Illumina, Luminex) can have different binding agents (specific to different target molecules). As a non-limiting example, a bead can be conjugated to an aptamer used to detect the presence (quantitatively or qualitatively) of an antigen of interest, or it can also be used to isolate a component present in a selected biological sample (e.g., cell, cell-fragment or vesicle comprising the target molecule to which the aptamer is configured to bind or associate). Any molecule of organic origin can be successfully conjugated to a polystyrene bead through use of commercially available kits.

Binding agents against biomarkers of interest can be used with various bead based substrates, including but not limited to magnetic capture method, fluorescence activated cell sorting (FACS) or laser cytometry. Magnetic capture methods can include, but are not limited to, the use of magnetically activated cell sorter (MACS) microbeads or magnetic columns. Examples of bead or particle based methods that can be used to assess vesicles isolated using the methods provided herein include the methods and bead systems described in U.S. Pat. Nos. 4,551,435, 4,795,698, 4,925,788, 5,108,933, 5,186,827, 5,200,084 or 5,158,871; 7,399,632; 8,124,015; 8,008,019; 7,955,802; 7,445,844; 7,274,316; 6,773,812; 6,623,526; 6,599,331; 6,057,107; 5,736,330; International Patent Publication No. WO/2012/174282; WO/1993/022684.

Flow Cytometry

Isolation or detection of circulating biomarkers, e.g., protein antigens, from a biological sample, or of the biomarker-comprising cells, cell fragments or vesicles may also be achieved using a flow cytometry process. As a non-limiting example, an antibody or aptamer is used in an assay comprising using a particle such as a bead or microsphere. Binding agents may be conjugated to the particle. Flow cytometry can be used for sorting microscopic particles suspended in a stream of fluid. As particles pass through they can be selectively charged and on their exit can be deflected into separate paths of flow. It is therefore possible to separate populations from an original mix, such as a biological sample, with a high degree of accuracy and speed. Flow cytometry allows simultaneous multiparametric analysis of the physical and/or chemical characteristics of single cells flowing through an optical/electronic detection apparatus. A beam of light, usually laser light, of a single frequency (color) is directed onto a hydrodynamically focused stream of fluid. A number of detectors are aimed at the point where the stream passes through the light beam; one in line with the light beam (Forward Scatter or FSC) and several perpendicular to it (Side Scatter or SSC) and one or more fluorescent detectors.

Each suspended particle passing through the beam scatters the light in some way, and fluorescent chemicals in the particle may be excited into emitting light at a lower frequency than the light source. This combination of scattered and fluorescent light is picked up by the detectors, and by analyzing fluctuations in brightness at each detector (one for each fluorescent emission peak), it is possible to deduce various facts about the physical and chemical structure of each individual particle. FSC correlates with the cell size and SSC depends on the inner complexity of the particle, such as shape of the nucleus, the amount and type of cytoplasmic granules or the membrane roughness. Some flow cytometers have eliminated the need for fluorescence and use only light scatter for measurement.

Flow cytometers can analyze several thousand particles every second in “real time” and can actively separate out and isolate particles having specified properties. They offer high-throughput automated quantification, and separation, of the set parameters for a high number of single cells during each analysis session. Flow cytometers can have multiple lasers and fluorescence detectors, allowing multiple labels to be used to more precisely specify a target population by their phenotype. Thus, a flow cytometer, such as a multicolor flow cytometer, can be used to detect one or more vesicles with multiple fluorescent labels or colors. In some embodiments, the flow cytometer can also sort or isolate different vesicle populations, such as by size or by different markers.

The flow cytometer may have one or more lasers, such as 1, 2, 3, 4, 5, 6, 7, 8, 9, 10 or more lasers. In some embodiments, the flow cytometer can detect more than one color or fluorescent label, such as at least 2, 3, 4, 5, 6, 7, 8, 9, 10, 11, 12, 13, 14, 15, 16, 17, 18, 19, or 20 different colors or fluorescent labels. For example, the flow cytometer can have at least 2, 3, 4, 5, 6, 7, 8, 9, 10, 11, 12, 13, 14, 15, 16, 17, 18, 19, or 20 fluorescence detectors.

Examples of commercially available flow cytometers that can be used to detect or analyze one or more vesicles, to sort or separate different populations of vesicles, include, but are not limited to the MoFlo™ XDP Cell Sorter (Beckman Coulter, Brea, Calif.), MoFlo™ Legacy Cell Sorter (Beckman Coulter, Brea, Calif.), BD FACSAria™ Cell Sorter (BD Biosciences, San Jose, Calif.), BD™ LSRII (BD Biosciences, San Jose, Calif.), and BD FACSCalibur™ (BD Biosciences, San Jose, Calif.). Use of multicolor or multi-fluor cytometers can be used in multiplex analysis of vesicles, as further described below. In some embodiments, the flow cytometer can sort, and thereby collect or sort more than one population of vesicles based one or more characteristics. For example, two populations of vesicles differ in size, such that the vesicles within each population have a similar size range and can be differentially detected or sorted. In some embodiments, two different populations of vesicles are differentially labeled.

The data resulting from flow-cytometers can be plotted in 1 dimension to produce histograms or seen in 2 dimensions as dot plots or in 3 dimensions with newer software. The regions on these plots can be sequentially separated by a series of subset extractions which are termed gates. Specific gating protocols exist for diagnostic and clinical purposes especially in relation to hematology. The plots are often made on logarithmic scales. Because different fluorescent dye's emission spectra overlap, signals at the detectors have to be compensated electronically as well as computationally. Fluorophores for labeling biomarkers may include those described in Ormerod, Flow Cytometry 2nd ed., Springer-Verlag, New York (1999), and in Nida et al., Gynecologic Oncology 2005; 4 889-894 which is incorporated herein by reference. In a multiplexed assay, including but not limited to a flow cytometry assay, one or more different target molecules can be assessed, wherein at least one of the target molecules is a microvesicle surface antigen.

Microfluidics

Binding agents to a biomarker of interest can be disposed on any useful planar or bead substrate. In some embodiments, a binding agent is disposed on a microfluidic device, thereby facilitating assessing, characterizing or isolating a component of a biological sample comprising a polypeptide antigen of interest or a functional fragment thereof. For example, the circulating antigen or a cell, cell fragment or cell-derived vesicles comprising the antigen can be assessed using an antibody or aptamer to a vesicle biomarker of interest (alternatively along with additional binding agents). Microfluidic devices, which may also be referred to as “lab-on-a-chip” systems, biomedical micro-electro-mechanical systems (bioMEMs), or multicomponent integrated systems, can be used for isolating and analyzing a vesicle. Such systems miniaturize and compartmentalize processes that allow for binding of vesicles, detection of biosignatures, and other processes.

A microfluidic device can also be used for isolation of a vesicle through size differential or affinity selection. For example, a microfluidic device can use one more channels for isolating a vesicle from a biological sample based on size or by using one or more binding agents for isolating a vesicle from a biological sample. A biological sample can be introduced into one or more microfluidic channels, which selectively allows the passage of a vesicle. The selection can be based on a property of the vesicle, such as the size, shape, deformability, or biosignature of the vesicle.

In some embodiments, a heterogeneous population of vesicles can be introduced into a microfluidic device, and one or more different homogeneous populations of vesicles can be obtained. For example, different channels can have different size selections or binding agents to select for different vesicle populations. Thus, a microfluidic device can isolate a plurality of vesicles wherein at least a subset of the plurality of vesicles comprises a different biosignature from another subset of the plurality of vesicles. For example, the microfluidic device can isolate at least 2, 3, 4, 5, 6, 7, 8, 9, 10, 15, 20, 25, 30, 40, 50, 60, 70, 80, 90, or 100 different subsets of vesicles, wherein each subset of vesicles comprises a different biosignature.

In some embodiments, the microfluidic device can comprise one or more channels that permit further enrichment or selection of a vesicle. A population of vesicles that has been enriched after passage through a first channel can be introduced into a second channel, which allows the passage of the desired vesicle or vesicle population to be further enriched, such as through one or more binding agents present in the second channel.

Array-based assays and bead-based assays can be used with microfluidic device. For example, the binding agent can be coupled to beads and the binding reaction between the beads and vesicle can be performed in a microfluidic device. Multiplexing can also be performed using a microfluidic device. Different compartments can comprise different binding agents for different populations of vesicles, where each population is of a different cell-of-origin specific vesicle population. In some embodiments, each population has a different biosignature. The hybridization reaction between the microsphere and vesicle can be performed in a microfluidic device and the reaction mixture can be delivered to a detection device. The detection device, such as a dual or multiple laser detection system can be part of the microfluidic system and can use a laser to identify each bead or microsphere by its color-coding, and another laser can detect the hybridization signal associated with each bead.

Any appropriate microfluidic device can be used in the methods of the invention. Examples of microfluidic devices that may be used, or adapted for use with vesicles, include but are not limited to those described in U.S. Pat. Nos. 7,591,936, 7,581,429, 7,579,136, 7,575,722, 7,568,399, 7,552,741, 7,544,506, 7,541,578, 7,518,726, 7,488,596, 7,485,214, 7,467,928, 7,452,713, 7,452,509, 7,449,096, 7,431,887, 7,422,725, 7,422,669, 7,419,822, 7,419,639, 7,413,709, 7,411,184, 7,402,229, 7,390,463, 7,381,471, 7,357,864, 7,351,592, 7,351,380, 7,338,637, 7,329,391, 7,323,140, 7,261,824, 7,258,837, 7,253,003, 7,238,324, 7,238,255, 7,233,865, 7,229,538, 7,201,881, 7,195,986, 7,189,581, 7,189,580, 7,189,368, 7,141,978, 7,138,062, 7,135,147, 7,125,711, 7,118,910, 7,118,661, 7,640,947, 7,666,361, 7,704,735; and International Patent Publication WO 2010/072410; each of which patents or applications are incorporated herein by reference in their entirety. Another example for use with methods disclosed herein is described in Chen et al., “Microfluidic isolation and transcriptome analysis of serum vesicles,” Lab on a Chip, Dec. 8, 2009 DOI: 10.1039/b916199f.

Other microfluidic devices for use with the invention include devices comprising elastomeric layers, valves and pumps, including without limitation those disclosed in U.S. Pat. Nos. 5,376,252, 6,408,878, 6,645,432, 6,719,868, 6,793,753, 6,899,137, 6,929,030, 7,040,338, 7,118,910, 7,144,616, 7,216,671, 7,250,128, 7,494,555, 7,501,245, 7,601,270, 7,691,333, 7,754,010, 7,837,946; U.S. Patent Application Nos. 2003/0061687, 2005/0084421, 2005/0112882, 2005/0129581, 2005/0145496, 2005/0201901, 2005/0214173, 2005/0252773, 2006/0006067; and EP Patent Nos. 0527905 and 1065378; each of which application is herein incorporated by reference. In some instances, much or all of the devices are composed of elastomeric material. Certain devices are designed to conduct thermal cycling reactions (e.g., PCR) with devices that include one or more elastomeric valves to regulate solution flow through the device. The devices can comprise arrays of reaction sites thereby allowing a plurality of reactions to be performed. Thus, the devices can be used to assess circulating microRNAs in a multiplex fashion, including microRNAs isolated from vesicles. In some embodiments, the microfluidic device comprises (a) a first plurality of flow channels formed in an elastomeric substrate; (b) a second plurality of flow channels formed in the elastomeric substrate that intersect the first plurality of flow channels to define an array of reaction sites, each reaction site located at an intersection of one of the first and second flow channels; (c) a plurality of isolation valves disposed along the first and second plurality of flow channels and spaced between the reaction sites that can be actuated to isolate a solution within each of the reaction sites from solutions at other reaction sites, wherein the isolation valves comprise one or more control channels that each overlay and intersect one or more of the flow channels; and (d) means for simultaneously actuating the valves for isolating the reaction sites from each other. Various modifications to the basic structure of the device are envisioned within the scope of the invention. MicroRNAs can be detected in each of the reaction sites by using PCR methods. For example, the method can comprise the steps of the steps of: (i) providing a microfluidic device, the microfluidic device comprising: a first fluidic channel having a first end and a second end in fluid communication with each other through the channel; a plurality of flow channels, each flow channel terminating at a terminal wall; wherein each flow channel branches from and is in fluid communication with the first fluidic channel, wherein an aqueous fluid that enters one of the flow channels from the first fluidic channel can flow out of the flow channel only through the first fluidic channel; and, an inlet in fluid communication with the first fluidic channel, the inlet for introducing a sample fluid; wherein each flow channel is associated with a valve that when closed isolates one end of the flow channel from the first fluidic channel, whereby an isolated reaction site is formed between the valve and the terminal wall; a control channel; wherein each the valve is a deflectable membrane which is deflected into the flow channel associated with the valve when an actuating force is applied to the control channel, thereby closing the valve; and wherein when the actuating force is applied to the control channel a valve in each of the flow channels is closed, so as to produce the isolated reaction site in each flow channel; (ii) introducing the sample fluid into the inlet, the sample fluid filling the flow channels; (iii) actuating the valve to separate the sample fluid into the separate portions within the flow channels; (iv) amplifying the nucleic acid in the sample fluid; (v) analyzing the portions of the sample fluid to determine whether the amplifying produced the reaction. The sample fluid can contain an amplifiable nucleic acid target, e.g., a microRNA, and the conditions can be polymerase chain reaction (PCR) conditions, so that the reaction results in a PCR product being formed.

The microfluidic device can have one or more binding agents attached to a surface in a channel, or present in a channel. For example, the microchannel can have one or more capture agents, such as a capture agent for a tissue related antigen, one or more general microvesicle antigen (e.g., as listed in Table 3 as desired) or a cell-of-origin or cancer related antigen, including without limitation EpCam, CD9, CD63, CD81, B7H3, ICAM, STEAP, KLK2, SSX2, SSX4, PBP, SPDEF, and EGFR. The capture agent may be an aptamer. In some embodiments, a microchannel surface is treated with avidin and a capture agent, such as an antibody, that is biotinylated can be injected into the channel to bind the avidin. In other embodiments, the capture agents are present in chambers or other components of a microfluidic device. The capture agents can also be attached to beads that can be manipulated to move through the microfluidic channels. In some embodiments, the capture agents are attached to magnetic beads. The beads can be manipulated using magnets.

A biological sample can be flowed into the microfluidic device, or a microchannel, at rates such as at least about 1, 2, 3, 4, 5, 6, 7, 8, 9, 10, 11, 12, 13, 14, 15, 16, 17, 18, 19, 20, 25, 30, 35, 40, 45, or 50 μl per minute, such as between about 1-50, 5-40, 5-30, 3-20 or 5-15 μl per minute. One or more vesicles can be captured and directly detected in the microfluidic device. Alternatively, the captured vesicle may be released and exit the microfluidic device prior to analysis. In some embodiments, one or more captured vesicles are lysed in the microchannel and the lysate can be analyzed, e.g., to examine payload within the vesicles. Lysis buffer can be flowed through the channel and lyse the captured vesicles. For example, the lysis buffer can be flowed into the device or microchannel at rates such as at least about a, 2, 3, 4, 5, 6, 7, 8, 9, 10, 11, 12, 13, 14, 15, 16, 17, 18, 19, 20, 25, 26, 27, 28, 29, 30, 35, 40, 45, or 50 μl per minute, such as between about 1-50, 5-40, 10-30, 5-30 or 10-35 μl per minute. The lysate can be collected and analyzed, such as performing RT-PCR, PCR, mass spectrometry, Western blotting, or other assays, to detect one or more biomarkers of the vesicle.

Vesicle Isolation Techniques

One of skill will appreciate that various methods of sample treatment and isolating and concentrating circulating biomarkers such as vesicles can be combined as desired. For example, a biological sample can be treated to prevent aggregation, remove undesired particulate and/or deplete highly abundant proteins. The steps used can be chosen to optimize downstream analysis steps. Biomarkers such as vesicles can be isolated from the treated sample, preferably using the isolation method provided herein. Additional isolation steps can be employed if desired, e.g., by chromotography, centrifugation, density gradient, filtration, precipitation, or affinity techniques. Any number of the later steps can be combined, e.g., a sample could be subjected to one or more of chromotography, centrifugation, density gradient, filtration and precipitation in order to isolate or concentrate all or most microvesicles. In a subsequent step, affinity techniques, e.g., using binding agents to one or more target of interest, can be used to isolate or identify microvesicles carrying desired biomarker profiles. Microfluidic systems can be employed to perform some or all of these steps.

An non-limiting and exemplary isolation scheme for isolating and analysis of microvesicles includes the following: Plasma or serum collection->highly abundant protein removal->F68 isolation as provided herein->flow cytometry, aptamer library (see FIGS. 2A-F and related text), or particle-based assay.

Using the methods disclosed herein or known in the art, vesicles can be isolated or concentrated by at least about 2-fold, 3-fold, 1-fold, 2-fold, 3-fold, 4-fold, 5-fold, 6-fold, 7-fold, 8-fold, 9-fold, 10-fold, 12-fold, 15-fold, 20-fold, 25-fold, 30-fold, 35-fold, 40-fold, 45-fold, 50-fold, 55-fold, 60-fold, 65-fold, 70-fold, 75-fold, 80-fold, 90-fold, 95-fold, 100-fold, 110-fold, 120-fold, 125-fold, 130-fold, 140-fold, 150-fold, 160-fold, 170-fold, 175-fold, 180-fold, 190-fold, 200-fold, 225-fold, 250-fold, 275-fold, 300-fold, 325-fold, 350-fold, 375-fold, 400-fold, 425-fold, 450-fold, 475-fold, 500-fold, 525-fold, 550-fold, 575-fold, 600-fold, 625-fold, 650-fold, 675-fold, 700-fold, 725-fold, 750-fold, 775-fold, 800-fold, 825-fold, 850-fold, 875-fold, 900-fold, 925-fold, 950-fold, 975-fold, 1000-fold, 1500-fold, 2000-fold, 2500-fold, 3000-fold, 4000-fold, 5000-fold, 6000-fold, 7000-fold, 8000-fold, 9000-fold, or at least 10,000-fold. In some embodiments, the vesicles are isolated or concentrated concentrated by at least 1 order of magnitude, 2 orders of magnitude, 3 orders of magnitude, 4 orders of magnitude, 5 orders of magnitude, 6 orders of magnitude, 7 orders of magnitude, 8 orders of magnitude, 9 orders of magnitude, or 10 orders of magnitude or more.

Once concentrated or isolated, the circulating biomarkers can be assessed, e.g., in order to characterize a phenotype as described herein. In some embodiments, the concentration or isolation steps themselves shed light on the phenotype of interest. For example, affinity methods can detect the presence or level of specific biomarkers of interest.

### Phenotypes

Disclosed herein are products and processes for characterizing a phenotype using the methods and compositions of the invention. The term “phenotype” as used herein can mean any trait or characteristic that is attributed to a biomarker profile that is identified using in part or in whole the compositions and/or methods of the invention. For example, a phenotype can be a diagnostic, prognostic or theranostic determination based on a characterized biomarker profile for a sample obtained from a subject. A phenotype can be any observable characteristic or trait of, such as a disease or condition, a stage of a disease or condition, susceptibility to a disease or condition, prognosis of a disease stage or condition, a physiological state, or response/potential response to therapeutics. A phenotype can result from a subject's genetic makeup as well as the influence of environmental factors and the interactions between the two, as well as from epigenetic modifications to nucleic acid sequences.

A phenotype in a subject can be characterized by obtaining a biological sample from a subject and analyzing the sample using the compositions and/or methods of the invention. For example, characterizing a phenotype for a subject or individual can include detecting a disease or condition (including pre-symptomatic early stage detecting), determining a prognosis, diagnosis, or theranosis of a disease or condition, or determining the stage or progression of a disease or condition. Characterizing a phenotype can include identifying appropriate treatments or treatment efficacy for specific diseases, conditions, disease stages and condition stages, predictions and likelihood analysis of disease progression, particularly disease recurrence, metastatic spread or disease relapse. A phenotype can also be a clinically distinct type or subtype of a condition or disease, such as a cancer or tumor. Phenotype determination can also be a determination of a physiological condition, or an assessment of organ distress or organ rejection, such as post-transplantation. The compositions and methods described herein allow assessment of a subject on an individual basis, which can provide benefits of more efficient and economical decisions in treatment.

In an aspect, the invention relates to the analysis of biomarkers such as microvesicles to provide a diagnosis, prognosis, and/or theranosis of a disease or condition. Theranostics includes diagnostic testing that provides the ability to affect therapy or treatment of a disease or disease state. Theranostics testing provides a theranosis in a similar manner that diagnostics or prognostic testing provides a diagnosis or prognosis, respectively. As used herein, theranostics encompasses any desired form of therapy related testing, including predictive medicine, personalized medicine, integrated medicine, pharmacodiagnostics and Dx/Rx partnering. Therapy related tests can be used to predict and assess drug response in individual subjects, i.e., to provide personalized medicine. Predicting a drug response can be determining whether a subject is a likely responder or a likely non-responder to a candidate therapeutic agent, e.g., before the subject has been exposed or otherwise treated with the treatment. Assessing a drug response can be monitoring a response to a drug, e.g., monitoring the subject's improvement or lack thereof over a time course after initiating the treatment. Therapy related tests are useful to select a subject for treatment who is particularly likely to benefit from the treatment or to provide an early and objective indication of treatment efficacy in an individual subject. Thus, analysis using the compositions and methods of the invention may indicate that treatment should be altered to select a more promising treatment, thereby avoiding the great expense of delaying beneficial treatment and avoiding the financial and morbidity costs of administering an ineffective drug(s).

Thus, the compositions and methods of the invention may help predict whether a subject is likely to respond to a treatment for a disease or disorder. Characterizating a phenotype includes predicting the responder/non-responder status of the subject, wherein a responder responds to a treatment for a disease and a non-responder does not respond to the treatment. Biomarkers such as microvesicles can be analyzed in the subject and compared against that of previous subjects that were known to respond or not to a treatment. If the biomarker profile in the subject more closely aligns with that of previous subjects that were known to respond to the treatment, the subject can be characterized, or predicted, as a responder to the treatment. Similarly, if the biomarker profile in the subject more closely aligns with that of previous subjects that did not respond to the treatment, the subject can be characterized, or predicted as a non-responder to the treatment. The treatment can be for any appropriate disease, disorder or other condition, including without limitation those disclosed herein.

In some embodiments, the phenotype comprises a disease or condition such as those listed in Table 1 or provided elsewhere herein. For example, the phenotype can comprise detecting the presence of or likelihood of developing a tumor, neoplasm, or cancer, or characterizing the tumor, neoplasm, or cancer (e.g., stage, grade, aggressiveness, likelihood of metastatis or recurrence, etc). Cancers that can be detected or assessed by methods or compositions described herein include, but are not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, and Burkitt's lymphoma.

The phenotype can be a premalignant condition, such as actinic keratosis, atrophic gastritis, leukoplakia, erythroplasia, Lymphomatoid Granulomatosis, preleukemia, fibrosis, cervical dysplasia, uterine cervical dysplasia, xeroderma pigmentosum, Barrett's Esophagus, colorectal polyp, or other abnormal tissue growth or lesion that is likely to develop into a malignant tumor. Transformative viral infections such as HIV and HPV also present phenotypes that can be assessed according to the invention.

A cancer characterized by the methods of the invention can comprise, without limitation, a carcinoma, a sarcoma, a lymphoma or leukemia, a germ cell tumor, a blastoma, or other cancers. Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. Lymphoma and leukemia include without limitation chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical hodgkin lymphomas (nodular sclerosis, mixed cellularity, lymphocyte-rich, lymphocyte depleted or not depleted), and nodular lymphocyte-predominant hodgkin lymphoma. Germ cell tumors include without limitation germinoma, dysgerminoma, seminoma, nongerminomatous germ cell tumor, embryonal carcinoma, endodermal sinus turmor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In a further embodiment, the cancer under analysis may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemia, lymphoma, myeloma, or a solid tumor.

In embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom macroglobulinemia; or Wilm's tumor. The methods of the invention can be used to characterize these and other cancers. Thus, characterizing a phenotype can be providing a diagnosis, prognosis or theranosis of one of the cancers disclosed herein.

In some embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. The methods of the invention can be used to characterize these and other cancers. Thus, characterizing a phenotype can be providing a diagnosis, prognosis or theranosis of one of the cancers disclosed herein.

The phenotype can also be an inflammatory disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis.

The phenotype can also comprise a cardiovascular disease, such as atherosclerosis, congestive heart failure, vulnerable plaque, stroke, or ischemia. The cardiovascular disease or condition can be high blood pressure, stenosis, vessel occlusion or a thrombotic event.

The phenotype can also comprise a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neurospsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The phenotype may also be a condition such as fibromyalgia, chronic neuropathic pain, or peripheral neuropathic pain.

The phenotype may also comprise an infectious disease, such as a bacterial, viral or yeast infection. For example, the disease or condition may be Whipple's Disease, Prion Disease, cirrhosis, methicillin-resistant Staphylococcus aureus, HIV, hepatitis, syphilis, meningitis, malaria, tuberculosis, or influenza. Viral proteins, such as HIV or HCV-like particles can be assessed in a vesicle, to characterize a viral condition.

The phenotype can also comprise a perinatal or pregnancy related condition (e.g. preeclampsia or preterm birth), metabolic disease or condition, such as a metabolic disease or condition associated with iron metabolism. For example, hepcidin can be assayed in a vesicle to characterize an iron deficiency. The metabolic disease or condition can also be diabetes, inflammation, or a perinatal condition.

The compositions and methods of the invention can be used to characterize these and other diseases and disorders that can be assessed via biomarkers. Thus, characterizing a phenotype can be providing a diagnosis, prognosis or theranosis of one of the diseases and disorders disclosed herein.

### Subject

One or more phenotypes of a subject can be determined by analyzing one or more vesicles, such as vesicles, in a biological sample obtained from the subject. A subject or patient can include, but is not limited to, mammals such as bovine, avian, canine, equine, feline, ovine, porcine, or primate animals (including humans and non-human primates). A subject can also include a mammal of importance due to being endangered, such as a Siberian tiger; or economic importance, such as an animal raised on a farm for consumption by humans, or an animal of social importance to humans, such as an animal kept as a pet or in a zoo. Examples of such animals include, but are not limited to, carnivores such as cats and dogs; swine including pigs, hogs and wild boars; ruminants or ungulates such as cattle, oxen, sheep, giraffes, deer, goats, bison, camels or horses. Also included are birds that are endangered or kept in zoos, as well as fowl and more particularly domesticated fowl, i.e. poultry, such as turkeys and chickens, ducks, geese, guinea fowl. Also included are domesticated swine and horses (including race horses). In addition, any animal species connected to commercial activities are also included such as those animals connected to agriculture and aquaculture and other activities in which disease monitoring, diagnosis, and therapy selection are routine practice in husbandry for economic productivity and/or safety of the food chain.

The subject can have a pre-existing disease or condition, such as cancer. Alternatively, the subject may not have any known pre-existing condition. The subject may also be non-responsive to an existing or past treatment, such as a treatment for cancer.

### Samples

A sample used and/or assessed via the compositions and methods of the invention includes any relevant biological sample that can be used for biomarker assessment, including without limitation sections of tissues such as biopsy or tissue removed during surgical or other procedures, bodily fluids, autopsy samples, frozen sections taken for histological purposes, and cell cultures. Such samples include blood and blood fractions or products (e.g., serum, buffy coat, plasma, platelets, red blood cells, and the like), sputum, malignant effusion, cheek cells tissue, cultured cells (e.g., primary cultures, explants, and transformed cells), stool, urine, other biological or bodily fluids (e.g., prostatic fluid, gastric fluid, intestinal fluid, renal fluid, lung fluid, cerebrospinal fluid, and the like), etc. The sample can comprise biological material that is a fresh frozen & formalin fixed paraffin embedded (FFPE) block, formalin-fixed paraffin embedded, or is within an RNA preservative+formalin fixative. More than one sample of more than one type can be used for each patient.

The sample used in the methods described herein can be a formalin fixed paraffin embedded (FFPE) sample. The FFPE sample can be one or more of fixed tissue, unstained slides, bone marrow core or clot, core needle biopsy, malignant fluids and fine needle aspirate (FNA). In some embodiments, the fixed tissue comprises a tumor containing formalin fixed paraffin embedded (FFPE) block from a surgery or biopsy. In some embodiments, the unstained slides comprise unstained, charged, unbaked slides from a paraffin block. In some embodiments, bone marrow core or clot comprises a decalcified core. A formalin fixed core and/or clot can be paraffin-embedded. In still some embodiments, the core needle biopsy comprises 1, 2, 3, 4, 5, 6, 7, 8, 9, 10 or more, e.g., 3-4, paraffin embedded biopsy samples. An 18 gauge needle biopsy can be used. The malignant fluid can comprise a sufficient volume of fresh pleural/ascitic fluid to produce a 5×5×2 mm cell pellet. The fluid can be formalin fixed in a paraffin block. In some embodiments, the core needle biopsy comprises 1, 2, 3, 4, 5, 6, 7, 8, 9, 10 or more, e.g., 4-6, paraffin embedded aspirates.

A sample may be processed according to techniques understood by those in the art. A sample can be without limitation fresh, frozen or fixed cells or tissue. In some embodiments, a sample comprises formalin-fixed paraffin-embedded (FFPE) tissue, fresh tissue or fresh frozen (FF) tissue. A sample can comprise cultured cells, including primary or immortalized cell lines derived from a subject sample. A sample can also refer to an extract from a sample from a subject. For example, a sample can comprise DNA, RNA or protein extracted from a tissue or a bodily fluid. Many techniques and commercial kits are available for such purposes. The fresh sample from the individual can be treated with an agent to preserve RNA prior to further processing, e.g., cell lysis and extraction. Samples can include frozen samples collected for other purposes. Samples can be associated with relevant information such as age, gender, and clinical symptoms present in the subject; source of the sample; and methods of collection and storage of the sample. A sample is typically obtained from a subject.

A biopsy comprises the process of removing a tissue sample for diagnostic or prognostic evaluation, and to the tissue specimen itself. Any biopsy technique known in the art can be applied to the molecular profiling methods of the present invention. The biopsy technique applied can depend on the tissue type to be evaluated (e.g., colon, prostate, kidney, bladder, lymph node, liver, bone marrow, blood cell, lung, breast, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. Molecular profiling can use a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

Standard molecular biology techniques known in the art and not specifically described are generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) can be carried out generally as in PCR Protocols: A Guide to Methods and Applications, Academic Press, San Diego, Calif. (1990).

The biological sample assessed using the compositions and methods of the invention can be any useful bodily or biological fluid, including but not limited to peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids, cells, cell culture, or a cell culture supernatant. A biological sample may also include the blastocyl cavity, umbilical cord blood, or maternal circulation which may be of fetal or maternal origin. The biological sample may also be a cell culture, tissue sample or biopsy from which vesicles and other circulating biomarkers may be obtained. For example, cells of interest can be cultured and vesicles isolated from the culture. In various embodiments, biomarkers or more particularly biosignatures disclosed herein can be assessed directly from such biological samples (e.g., identification of presence or levels of nucleic acid or polypeptide biomarkers or functional fragments thereof) using various methods, such as extraction of nucleic acid molecules from blood, plasma, serum or any of the foregoing biological samples, use of protein or antibody arrays to identify polypeptide (or functional fragment) biomarker(s), as well as other array, sequencing, PCR and proteomic techniques known in the art for identification and assessment of nucleic acid and polypeptide molecules. In addition, one or more components present in such samples can be first isolated or enriched and further processed to assess the presence or levels of selected biomarkers, to assess a given biosignature (e.g., isolated microvesicles prior to profiling for protein and/or nucleic acid biomarkers).

Table 1 presents a non-limiting listing of diseases, conditions, or biological states and corresponding biological samples that may be used for analysis according to the methods of the invention.

TABLE 1
Examples of Biological Samples for Various Diseases, Conditions, or Biological States

| Illustrative Disease, Condition or Biological | |
|---|---|
| State | Illustrative Biological Samples |
| Cancers/neoplasms affecting the following tissue | Tumor, blood, serum, plasma, cerebrospinal fluid |
| types/bodily systems: breast, lung, ovarian, colon, | (CSF), urine, sputum, ascites, synovial fluid, |
| rectal, prostate, pancreatic, brain, bone, connective | semen, nipple aspirates, saliva, bronchoalveolar |
| tissue, glands, skin, lymph, nervous system, | lavage fluid, tears, oropharyngeal washes, feces, |
| endocrine, germ cell, genitourinary, | peritoneal fluids, pleural effusion, sweat, tears, |
| hematologic/blood, bone marrow, muscle, eye, | aqueous humor, pericardial fluid, lymph, chyme, |
| esophageal, fat tissue, thyroid, pituitary, spinal | chyle, bile, stool water, amniotic fluid, breast milk, |
| cord, bile duct, heart, gall bladder, bladder, testes, | pancreatic juice, cerumen, Cowper's fluid or pre- |
| cervical, endometrial, renal, ovarian, | ejaculatory fluid, female ejaculate, interstitial fluid, |
| digestive/gastrointestinal, stomach, head and neck, | menses, mucus, pus, sebum, vaginal lubrication, |
| liver, leukemia, respiratory/thorasic, cancers of | vomit |
| unknown primary (CUP) | |
| Neurodegenerative/neurological disorders: | Blood, serum, plasma, CSF, urine |
| Parkinson's disease, Alzheimer's Disease and | |
| multiple sclerosis, Schizophrenia, and bipolar | |
| disorder, spasticity disorders, epilepsy | |
| Cardiovascular Disease: atherosclerosis, | Blood, serum, plasma, CSF, urine |
| cardiomyopathy, endocarditis, vunerable plaques, | |
| infection | |
| Stroke: ischemic, intracerebral hemorrhage, | Blood, serum, plasma, CSF, urine |
| subarachnoid hemorrhage, transient ischemic | |
| attacks (TIA) | |
| Pain disorders: peripheral neuropathic pain and | Blood, serum, plasma, CSF, urine |
| chronic neuropathic pain, and fibromyalgia, | |
| Autoimmune disease: systemic and localized | Blood, serum, plasma, CSF, urine, synovial fluid |
| diseases, rheumatic disease, Lupus, Sjogren's | |
| syndrome | |
| Digestive system abnormalities: Barrett's | Blood, serum, plasma, CSF, urine |
| esophagus, irritable bowel syndrome, ulcerative | |
| colitis, Crohn's disease, Diverticulosis and | |
| Diverticulitis, Celiac Disease | |
| Endocrine disorders: diabetes mellitus, various | Blood, serum, plasma, CSF, urine |
| forms of Thyroiditis, adrenal disorders, pituitary | |
| disorders | |
| Diseases and disorders of the skin: psoriasis | Blood, serum, plasma, CSF, urine, synovial fluid, |
| | tears |
| Urological disorders: benign prostatic hypertrophy | Blood, serum, plasma, urine |
| (BPH), polycystic kidney disease, interstitial | |
| cystitis | |
| Hepatic disease/injury: Cirrhosis, induced | Blood, serum, plasma, urine |
| hepatotoxicity (due to exposure to natural or | |
| synthetic chemical sources) | |
| Kidney disease/injury: acute, sub-acute, chronic | Blood, serum, plasma, urine |
| conditions, Podocyte injury, focal segmental | |
| glomerulosclerosis | |
| Endometriosis | Blood, serum, plasma, urine, vaginal fluids |
| Osteoporosis | Blood, serum, plasma, urine, synovial fluid |
| Pancreatitis | Blood, serum, plasma, urine, pancreatic juice |
| Asthma | Blood, serum, plasma, urine, sputum, bronchiolar |
| | lavage fluid |
| Allergies | Blood, serum, plasma, urine, sputum, bronchiolar |
| | lavage fluid |
| Prion-related diseases | Blood, serum, plasma, CSF, urine |
| Viral Infections: HIV/AIDS | Blood, serum, plasma, urine |
| Sepsis | Blood, serum, plasma, urine, tears, nasal lavage |
| Organ rejection/transplantation | Blood, serum, plasma, urine, various lavage fluids |
| Differentiating conditions: adenoma versus | Blood, serum, plasma, urine, sputum, feces, colonic |
| hyperplastic polyp, irritable bowel syndrome (IBS) | lavage fluid |
| versus normal, classifying Dukes stages A, B, C, | |
| and/or D of colon cancer, adenoma with low-grade | |
| hyperplasia versus high-grade hyperplasia, | |
| adenoma versus normal, colorectal cancer versus | |
| normal, IBS versus. ulcerative colitis (UC) versus | |
| Crohn's disease (CD), | |
| Pregnancy related physiological states, conditions, | Maternal serum, plasma, amniotic fluid, cord blood |
| or affiliated diseases: genetic risk, adverse | |
| pregnancy outcomes | |

The methods of the invention can be used to characterize a phenotype using a blood sample or blood derivative. Blood derivatives include plasma and serum. Blood plasma is the liquid component of whole blood, and makes up approximately 55% of the total blood volume. It is composed primarily of water with small amounts of minerals, salts, ions, nutrients, and proteins in solution. In whole blood, red blood cells, leukocytes, and platelets are suspended within the plasma. Blood serum refers to blood plasma without fibrinogen or other clotting factors (i.e., whole blood minus both the cells and the clotting factors).

The biological sample may be obtained through a third party, such as a party not performing the analysis of the biomarkers, whether direct assessment of a biological sample or by profiling one or more vesicles obtained from the biological sample. For example, the sample may be obtained through a clinician, physician, or other health care manager of a subject from which the sample is derived. Alternatively, the biological sample may obtained by the same party analyzing the vesicle. In addition, biological samples be assayed, are archived (e.g., frozen) or otherwise stored in under preservative conditions.

Furthermore, a biological sample can comprise a vesicle or cell membrane fragment that is derived from a cell of origin and available extracellularly in a subject's biological fluid or extracellular milieu.

Methods of the invention can include assessing one or more vesicles, including assessing vesicle populations. A vesicle, as used herein, is a membrane vesicle that is shed from cells. Vesicles or membrane vesicles include without limitation: circulating microvesicles (cMVs), microvesicle, exosome, nanovesicle, dexosome, bleb, blebby, prostasome, microparticle, intralumenal vesicle, membrane fragment, intralumenal endosomal vesicle, endosomal-like vesicle, exocytosis vehicle, endosome vesicle, endosomal vesicle, apoptotic body, multivesicular body, secretory vesicle, phospholipid vesicle, liposomal vesicle, argosome, texasome, secresome, tolerosome, melanosome, oncosome, or exocytosed vehicle. Furthermore, although vesicles may be produced by different cellular processes, the methods of the invention are not limited to or reliant on any one mechanism, insofar as such vesicles are present in a biological sample and are capable of being characterized by the methods disclosed herein. Unless otherwise specified, methods that make use of a species of vesicle can be applied to other types of vesicles. Vesicles comprise spherical structures with a lipid bilayer similar to cell membranes which surrounds an inner compartment which can contain soluble components, sometimes referred to as the payload. In some embodiments, the methods of the invention make use of exosomes, which are small secreted vesicles of about 40-100 nm in diameter. For a review of membrane vesicles, including types and characterizations, see Thery et al., Nat Rev Immunol. 2009 August; 9(8):581-93. Some properties of different types of vesicles include those in Table 2:

TABLE 2
Vesicle Properties

| | | | | | | | Membrane | | Exosome- | | Apoptotic | |
|---|---|---|---|---|---|---|---|---|---|---|---|---|
| Feature | Exosomes | | Microvesicles | | Ectosomes | | particles | | like vesicles | | vesicles | |
| Size | 50-100 | nm | 100-1,000 | nm | 50-200 | nm | 50-80 | nm | 20-50 | nm | 50-500 | nm |
| Density in | 1.13-1.19 | g/ml | | | | | 1.04-1.07 | g/ml | 1.1 | g/ml | 1.16-1.28 | g/ml |
| sucrose | | | | | | | | | | | | |

| EM | Cup shape | | Irregular | | Bilamellar | | Round | | Irregular | | Heterogeneous |
|---|---|---|---|---|---|---|---|---|---|---|---|
| appearance | | | shape, | | round | | | | shape | | |
| | | | electron | | structures | | | | | | |
| | | | dense | | | | | | | | |
| Sedimentation | 100,000 | g | 10,000 | g | 160,000-200,000 | g | 100,000-200,000 | g | 175,000 | g | 1,200 g, 10,000 g, |
| | | | | | | | | | | | 100,000 g |

| | | | | |
|---|---|---|---|---|
| Lipid | Enriched in | Expose PPS | Enriched in | No lipid |
| composition | cholesterol, | | cholesterol | rafts |
| | sphingomyelin | | and | |
| | and ceramide; | | diacylglycerol; | |
| | contains lipid | | expose PPS | |
| | rafts; expose | | | |
| | PPS | | | |

| Major protein | Tetraspanins | Integrins, | CR1 and | CD133; no | TNFRI | Histones |
|---|---|---|---|---|---|---|
| markers | (e.g., CD63, | selectins and | proteolytic | CD63 | | |
| | CD9), Alix, | CD40 ligand | enzymes; no | | | |
| | TSG101 | | CD63 | | | |
| Intracellular | Internal | Plasma | Plasma | Plasma | | |
| origin | compartments | membrane | membrane | membrane | | |
| | (endosomes) | | | | | |

Abbreviations:
phosphatidylserine (PPS);
electron microscopy (EM)

Vesicles include shed membrane bound particles, or “microparticles,” that are derived from either the plasma membrane or an internal membrane. Vesicles can be released into the extracellular environment from cells. Cells releasing vesicles include without limitation cells that originate from, or are derived from, the ectoderm, endoderm, or mesoderm. The cells may have undergone genetic, environmental, and/or any other variations or alterations. For example, the cell can be tumor cells. A vesicle can reflect any changes in the source cell, and thereby reflect changes in the originating cells, e.g., cells having various genetic mutations. In one mechanism, a vesicle is generated intracellularly when a segment of the cell membrane spontaneously invaginates and is ultimately exocytosed (see for example, Keller et al., Immunol. Lett. 107 (2): 102-8 (2006)). Vesicles also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the vesicle lumen, including but not limited to tumor-derived microRNAs or intracellular proteins. Blebs and blebbing are further described in Charras et al., Nature Reviews Molecular and Cell Biology, Vol. 9, No. 11, p. 730-736 (2008). A vesicle shed into circulation or bodily fluids from tumor cells may be referred to as a “circulating tumor-derived vesicle.” When such vesicle is an exosome, it may be referred to as a circulating-tumor derived exosome (CTE). In some instances, a vesicle can be derived from a specific cell of origin. CTE, as with a cell-of-origin specific vesicle, typically have one or more unique biomarkers that permit isolation of the CTE or cell-of-origin specific vesicle, e.g., from a bodily fluid and sometimes in a specific manner. For example, a cell or tissue specific markers are used to identify the cell of origin. Examples of such cell or tissue specific markers are disclosed herein and can further be accessed in the Tissue-specific Gene Expression and Regulation (TiGER) Database, available at bioinfo.wilmer.jhu.edu/tiger/; Liu et al. (2008) TiGER: a database for tissue-specific gene expression and regulation. BMC Bioinformatics. 9:271; TissueDistributionDBs, available at genome.dkfz-heidelberg.de/menu/tissue_db/index.html.

A vesicle can have a diameter of greater than about 10 nm, 20 nm, or 30 nm. A vesicle can have a diameter of greater than 40 nm, 50 nm, 100 nm, 200 nm, 500 nm, 1000 nm, 1500 nm, 2000 nm or greater than 10,000 nm. A vesicle can have a diameter of about 20-2000 nm, about 20-1500 nm, about 30-1000 nm, about 30-800 nm, about 30-200 nm, or about 30-100 nm. In some embodiments, the vesicle has a diameter of less than 10,000 nm, 2000 nm, 1500 nm, 1000 nm, 800 nm, 500 nm, 200 nm, 100 nm, 50 nm, 40 nm, 30 nm, 20 nm or less than 10 nm. As used herein the term “about” in reference to a numerical value means that variations of 10% above or below the numerical value are within the range ascribed to the specified value. Typical sizes for various types of vesicles are shown in Table 2. Vesicles can be assessed to measure the diameter of a single vesicle or any number of vesicles. For example, the range of diameters of a vesicle population or an average diameter of a vesicle population can be determined. Vesicle diameter can be assessed using methods known in the art, e.g., imaging technologies such as electron microscopy. In some embodiments, a diameter of one or more vesicles is determined using optical particle detection. See, e.g., U.S. Pat. No. 7,751,053, entitled “Optical Detection and Analysis of Particles” and issued Jul. 6, 2010; and U.S. Pat. No. 7,399,600, entitled “Optical Detection and Analysis of Particles” and issued Jul. 15, 2010.

In some embodiments, the methods of the invention comprise assessing vesicles directly such as in a biological sample without prior isolation, purification, or concentration from the biological sample. For example, the amount of vesicles in the sample can by itself provide a biosignature that provides a diagnostic, prognostic or theranostic determination. Alternatively, the vesicle in the sample may be isolated, captured, purified, or concentrated from a sample prior to analysis. As noted, isolation, capture or purification as used herein comprises partial isolation, partial capture or partial purification apart from other components in the sample. Vesicle isolation can be performed using various techniques as described herein, e.g., chromatography, filtration, centrifugation, flow cytometry, affinity capture (e.g., to a planar surface or bead), and/or using microfluidics. FIGS. 2E-F present an overview of a method of the invention for assessing microvesicles using an aptamer pool.

Vesicles such as exosomes can be assessed to provide a phenotypic characterization by comparing vesicle characteristics to a reference. In some embodiments, surface antigens on a vesicle are assessed. The surface antigens can provide an indication of the anatomical origin and/or cellular of the vesicles and other phenotypic information, e.g., tumor status. For example, wherein vesicles found in a patient sample, e.g., a bodily fluid such as blood, serum or plasma, are assessed for surface antigens indicative of colorectal origin and the presence of cancer. The surface antigens may comprise any informative biological entity that can be detected on the vesicle membrane surface, including without limitation surface proteins, lipids, carbohydrates, and other membrane components. For example, positive detection of colon derived vesicles expressing tumor antigens can indicate that the patient has colorectal cancer. As such, methods of the invention can be used to characterize any disease or condition associated with an anatomical or cellular origin, by assessing, for example, disease-specific and cell-specific biomarkers of one or more vesicles obtained from a subject.

In some embodiments, the methods of the invention comprise assessing one or more vesicle payload to provide a phenotypic characterization. The payload with a vesicle comprises any informative biological entity that can be detected as encapsulated within the vesicle, including without limitation proteins and nucleic acids, e.g., genomic or cDNA, mRNA, or functional fragments thereof, as well as microRNAs (miRs). In addition, methods of the invention are directed to detecting vesicle surface antigens (in addition or exclusive to vesicle payload) to provide a phenotypic characterization. For example, vesicles can be characterized by using binding agents (e.g., antibodies or aptamers) that are specific to vesicle surface antigens, and the bound vesicles can be further assessed to identify one or more payload components disclosed therein. As described herein, the levels of vesicles with surface antigens of interest or with payload of interest can be compared to a reference to characterize a phenotype. For example, overexpression in a sample of cancer-related surface antigens or vesicle payload, e.g., a tumor associated mRNA or microRNA, as compared to a reference, can indicate the presence of cancer in the sample. The biomarkers assessed can be present or absent, increased or reduced based on the selection of the desired target sample and comparison of the target sample to the desired reference sample. Non-limiting examples of target samples include: disease; treated/not-treated; different time points, such as a in a longitudinal study; and non-limiting examples of reference sample: non-disease; normal; different time points; and sensitive or resistant to candidate treatment(s).

### Microvesicle Isolation and Analysis

Sample Processing

A vesicle or a population of vesicles may be isolated, purified, concentrated or otherwise enriched prior to and/or during analysis. Unless otherwise specified, the terms “purified,” “isolated,” or similar as used herein in reference to vesicles or biomarker components are intended to include partial or complete purification or isolation of such components from a cell or organism. Analysis of a vesicle can include quantitiating the amount one or more vesicle populations of a biological sample. For example, a heterogeneous population of vesicles can be quantitated, or a homogeneous population of vesicles, such as a population of vesicles with a particular biomarker profile, a particular biosignature, or derived from a particular cell type can be isolated from a heterogeneous population of vesicles and quantitated. Analysis of a vesicle can also include detecting, quantitatively or qualitatively, one or more particular biomarker profile or biosignature of a vesicle, as described herein.

A vesicle can be stored and archived, such as in a bio-fluid bank and retrieved for analysis as desired. A vesicle may also be isolated from a biological sample that has been previously harvested and stored from a living or deceased subject. In addition, a vesicle may be isolated from a biological sample which has been collected as described in King et al., Breast Cancer Res 7(5): 198-204 (2005). A vesicle can be isolated from an archived or stored sample. Alternatively, a vesicle may be isolated from a biological sample and analyzed without storing or archiving of the sample. Furthermore, a third party may obtain or store the biological sample, or obtain or store the vesicle for analysis.

An enriched population of vesicles can be obtained from a biological sample. For example, vesicles may be concentrated or isolated from a biological sample using size exclusion chromatography, density gradient centrifugation, differential centrifugation, nanomembrane ultrafiltration, immunoabsorbent capture, affinity purification, microfluidic separation, or combinations thereof.

Size exclusion chromatography, such as gel permeation columns, centrifugation or density gradient centrifugation, and filtration methods can be used. For example, a vesicle can be isolated by differential centrifugation, anion exchange and/or gel permeation chromatography (for example, as described in U.S. Pat. Nos. 6,899,863 and 6,812,023), sucrose density gradients, organelle electrophoresis (for example, as described in U.S. Pat. No. 7,198,923), magnetic activated cell sorting (MACS), or with a nanomembrane ultrafiltration concentrator. Various combinations of isolation or concentration methods can be used.

Highly abundant proteins, such as albumin and immunoglobulin in blood samples, may hinder isolation of vesicles from a biological sample. For example, a vesicle can be isolated from a biological sample using a system that uses multiple antibodies that are specific to the most abundant proteins found in a biological sample, such as blood. Such a system can remove up to several proteins at once, thus unveiling the lower abundance species such as cell-of-origin specific vesicles. This type of system can be used for isolation of vesicles from biological samples such as blood, cerebrospinal fluid or urine. The isolation of vesicles from a biological sample may also be enhanced by high abundant protein removal methods as described in Chromy et al. J Proteome Res 2004; 3:1120-1127. In some embodiments, the isolation of vesicles from a biological sample may also be enhanced by removing serum proteins using glycopeptide capture as described in Zhang et al, Mol Cell Proteomics 2005; 4:144-155. In addition, vesicles from a biological sample such as urine may be isolated by differential centrifugation followed by contact with antibodies directed to cytoplasmic or anti-cytoplasmic epitopes as described in Pisitkun et al., Proc Natl Acad Sci USA, 2004; 101:13368-13373.

Plasma contains a large variety of proteins including albumin, immunoglobulins, and clotting proteins such as fibrinogen. About 60% of plasma protein comprises the protein albumin (e.g., human serum albumin or HSA), which contributes to osmotic pressure of plasma to assist in the transport of lipids and steroid hormones. Globulins make up about 35% of plasma proteins and are used in the transport of ions, hormones and lipids assisting in immune function. About 4% of plasma protein comprises fibrinogen which is essential in the clotting of blood and can be converted into the insoluble protein fibrin. Other types of blood proteins include: Prealbumin, Alpha 1 antitrypsin, Alpha 1 acid glycoprotein, Alpha 1 fetoprotein, Haptoglobin, Alpha 2 macroglobulin, Ceruloplasmin, Transferrin, complement proteins C3 and C4, Beta 2 microglobulin, Beta lipoprotein, Gamma globulin proteins, C-reactive protein (CRP), Lipoproteins (chylomicrons, VLDL, LDL, HDL), other globulins (types alpha, beta and gamma), Prothrombin and Mannose-binding lectin (MBL). Any of these proteins, including classes of proteins, or derivatives thereof (such as fibrin which is derived from the cleavage of fibrinogen) can be selectively depleted from a biological sample prior to further analysis performed on the sample. Without being bound by theory, removal of such background proteins may facilitate more sensitive, accurate, or precise detection of the biomarkers of interest in the sample.

Abundant proteins in blood or blood derivatives (e.g., plasma or serum) include without limitation albumin, IgG, transferrin, fibrinogen, IgA, α₂-Macroglobulin, IgM, α₁-Antitrypsin, complement C3, haptoglobulin, apolipoprotein Al, apolipoprotein A3, apolipoprotein B, α₁-Acid Glycoprotein, ceruloplasmin, complement C4, C1q, IgD, prealbumin (transthyretin), and plasminogen. Such proteins can be depleted using commercially available columns and kits. Examples of such columns comprise the Multiple Affinity Removal System from Agilent Technologies (Santa Clara, Calif.). This system include various cartridges designed to deplete different protein profiles, including the following cartridges with performance characteristics according to the manufacturer: Human 14, which eliminates approximately 94% of total protein (albumin, IgG, antitrypsin, IgA, transferrin, haptoglobin, fibrinogen, alpha2-macroglobulin, alpha1-acid glycoprotein (orosomucoid), IgM, apolipoprotein AI, apolipoprotein AII, complement C3 and transthyretin); Human 7, which eliminates approximately 85-90% of total protein (albumin, IgG, IgA, transferrin, haptoglobin, antitrypsin, and fibrinogen); Human 6, which eliminates approximately 85-90% of total protein (albumin, IgG, IgA, transferrin, haptoglobin, and antitrypsin); Human Albumin/IgG, which eliminates approximately 69% of total protein (albumin and IgG); and Human Albumin, which eliminates approximately 50-55% of total protein (albumin). The ProteoPrep® 20 Plasma Immunodepletion Kit from Sigma-Aldrich is intended to specifically remove the 20 most abundant proteins from human plasma or serum, which is about remove 97-98% of the total protein mass in plasma or serum (Sigma-Aldrich, St. Louis, Mo.). According to the manufacturer, the ProteoPrep® 20 removes: albumin, IgG, transferrin, fibrinogen, IgA, α₂-Macroglobulin, IgM, α₁-Antitrypsin, complement C3, haptoglobulin, apolipoprotein Al, A3 and B; α₁-Acid Glycoprotein, ceruloplasmin, complement C4, C1q; IgD, prealbumin, and plasminogen. Sigma-Aldrich also manufactures ProteoPrep® columns to remove albumin (HSA) and immunoglobulins (IgG). The ProteomeLab IgY-12 High Capacity Proteome Partitioning kits from Beckman Coulter (Fullerton, Calif.) are specifically designed to remove twelve highly abundant proteins (Albumin, IgG, Transferrin, Fibrinogen, IgA, α2-macroglobulin, IgM, α1-Antitrypsin, Haptoglobin, Orosomucoid, Apolipoprotein A-I, Apolipoprotein A-II) from the human biological fluids such as serum and plasma. Generally, such systems rely on immunodepletion to remove the target proteins, e.g., using small ligands and/or full antibodies. The PureProteome™ Human Albumin/Immunoglobulin Depletion Kit from Millipore (EMD Millipore Corporation, Billerica, Mass., USA) is a magnetic bead based kit that enables high depletion efficiency (typically >99%) of Albumin and all Immunoglobulins (i.e., IgG, IgA, IgM, IgE and IgD) from human serum or plasma samples. The ProteoExtract Albumin/IgG Removal Kit, also from Millipore, is designed to deplete >80% of albumin and IgG from body fluid samples. Other similar protein depletion products include without limitation the following: Aurum™ Affi-Gels Blue mini kit (Bio-Rad, Hercules, Calif., USA); Vivapure® anti-HSA/IgG kit (Sartorius Stedim Biotech, Goettingen, Germany), Qproteome albumin/IgG depletion kit (Qiagen, Hilden, Germany); Seppro® MIXED12-LC20 column (GenWay Biotech, San Diego, Calif., USA); Abundant Serum Protein Depletion Kit (Norgen Biotek Corp., Ontario, Canada); GBC Human Albumin/IgG/Transferrin 3 in 1 Depletion Column/Kit (Good Biotech Corp., Taiwan). These systems and similar systems can be used to remove abundant proteins from a biological sample, thereby improving the ability to detect low abundance circulating biomarkers such as proteins and vesicles.

Thromboplastin is a plasma protein aiding blood coagulation through conversion of prothrombin to thrombin. Thrombin in turn acts as a serine protease that converts soluble fibrinogen into insoluble strands of fibrin, as well as catalyzing many other coagulation-related reactions. Thus, thromboplastin is a protein that can be used to facilitate precipitation of fibrinogen/fibrin (blood clotting factors) out of plasma. In addition to or as an alternative to immunoaffinity protein removal, a blood sample can be treated with thromboplastin to deplete fibrinogen/fibrin. Thromboplastin removal can be performed in addition to or as an alternative to immunoaffinity protein removal as described above using methods known in the art. Precipitation of other proteins and/or other sample particulate can also improve detection of circulating biomarkers such as vesicles in a sample. For example, ammonium sulfate treatment as known in the art can be used to precipitate immunoglobulins and other highly abundant proteins.

In some embodiments, the invention provides a method of detecting a presence or level of one or more circulating biomarker such as a microvesicle in a biological sample, comprising: (a) providing a biological sample comprising or suspected to comprise the one or more circulating biomarker; (b) selectively depleting one or more abundant protein from the biological sample provided in step (a); (c) performing affinity selection of the one or more circulating biomarker from the sample depleted in step (b), thereby detecting the presence or level of one or more circulating biomarker. The biological sample may comprise a bodily fluid, e.g., peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, umbilical cord blood, or a derivative of any thereof. In some embodiments, the biological sample comprises peripheral blood, serum or plasma. Illustrative protocols and results from selectively depleting one or more abundant protein from blood plasma prior to vesicle detection can be found in Example 40 of International Patent Publication No. WO/2014/082083, filed Nov. 26, 2013, which patent publication is incorporated by reference herein in its entirety.

An abundant protein may comprise a protein in the sample that is present in the sample at a high enough concentration to potentially interfere with downstream processing or analysis. Typically, an abundant protein is not the target of any further analysis of the sample. The abundant protein may constitute at least 10⁻⁵, 10⁻⁴, 10⁻³, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 96, 97, 98 or at least 99% of the total protein mass in the sample. In some embodiments, the abundant protein is present at less than 10⁻⁵% of the total protein mass in the sample, e.g., in the case of a rare target of interest. As described herein, in the case of blood or a derivative thereof, the one or more abundant protein may comprise one or more of albumin, IgG, transferrin, fibrinogen, fibrin, IgA, α2-Marcroglobulin, IgM, α1-Antitrypsin, complement C3, haptoglobulin, apolipoprotein Al, A3 and B; α1-Acid Glycoprotein, ceruloplasmin, complement C4, C1q, IgD, prealbumin (transthyretin), plasminogen, a derivative of any thereof, and a combination thereof. The one or more abundant protein in blood or a blood derivative may also comprise one or more of Albumin, Immunoglobulins, Fibrinogen, Prealbumin, Alpha 1 antitrypsin, Alpha 1 acid glycoprotein, Alpha 1 fetoprotein, Haptoglobin, Alpha 2 macroglobulin, Ceruloplasmin, Transferrin, complement proteins C3 and C4, Beta 2 microglobulin, Beta lipoprotein, Gamma globulin proteins, C-reactive protein (CRP), Lipoproteins (chylomicrons, VLDL, LDL, HDL), other globulins (types alpha, beta and gamma), Prothrombin, Mannose-binding lectin (MBL), a derivative of any thereof, and a combination thereof.

In some embodiments, selectively depleting the one or more abundant protein comprises contacting the biological sample with thromboplastin to initiate precipitation of fibrin. The one or more abundant protein may also be depleted by immunoaffinity, precipitation, or a combination thereof. For example, the sample can be treated with thromboplastin to precipitate fibrin, and then the sample may be passed through a column to remove HSA, IgG, and other abundant proteins as desired.

“Selectively depleting” the one or more abundant protein comprises depleting the abundant protein from the sample at a higher percentage than depletion another entity in the sample, such as another protein or microvesicle, including a target of interest for downstream processing or analysis. Selectively depleting the one or more abundant protein may comprise depleting the abundant protein at a 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 10⁴-fold, 10⁵-fold, 10⁶-fold, 10⁷-fold, 10⁸-fold, 10⁹-fold, 10¹⁰-fold, 10¹¹-fold, 10¹²-fold, 10¹³-fold, 10¹⁴-fold, 10¹⁵-fold, 10¹⁶-fold, 10¹⁷-fold, 10¹⁸-fold, 10¹⁹-fold, 10²⁰-fold, or higher rate than another entity in the sample, such as another protein or microvesicle, including a target of interest for downstream processing or analysis. In some embodiments, there is little to no observable depletion of the target of interest as compared to the depletion of the abundant protein. In some embodiments, selectively depleting the one or more abundant protein from the biological sample comprises depleting at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the one or more abundant protein.

Removal of highly abundant proteins and other non-desired entities can further be facilitated with a non-stringent size exclusion step. For example, the sample can be processed using a high molecular weight cutoff size exclusion step to preferentially enrich high molecular weight vesicles apart from lower molecular weight proteins and other entities. In some embodiments, a sample is processed with a column (e.g., a gel filtration column) or filter having a molecular weight cutoff (MWCO) of 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, or greater than 10000 kiloDaltons (kDa). In some embodiments, a 700 kDa filtration column is used. In such a step, the vesicles will be retained or flow more slowly than the column or filter than the lower molecular weight entities. Such columns and filters are known in the art.

Isolation or enrichment of a vesicle from a biological sample can also be enhanced by use of sonication (for example, by applying ultrasound), detergents, other membrane-activating agents, or any combination thereof. For example, ultrasonic energy can be applied to a potential tumor site, and without being bound by theory, release of vesicles from a tissue can be increased, allowing an enriched population of vesicles that can be analyzed or assessed from a biological sample using one or more methods disclosed herein.

With methods of detecting circulating biomarkers as described here, e.g., antibody affinity isolation, the consistency of the results can be optimized as desired using various concentration or isolation procedures. Such steps can include agitation such as shaking or vortexing, different isolation techniques such as polymer based isolation, e.g., with PEG, and concentration to different levels during filtration or other steps. It will be understood by those in the art that such treatments can be applied at various stages of testing the vesicle containing sample. In some embodiments, the sample itself, e.g., a bodily fluid such as plasma or serum, is vortexed. In some embodiments, the sample is vortexed after one or more sample treatment step, e.g., vesicle isolation, has occurred. Agitation can occur at some or all appropriate sample treatment steps as desired. Additives can be introduced at the various steps to improve the process, e.g., to control aggregation or degradation of the biomarkers of interest.

The results can also be optimized as desirable by treating the sample with various agents. Such agents include additives to control aggregation and/or additives to adjust pH or ionic strength. Additives that control aggregation include blocking agents such as bovine serum albumin (BSA), milk or StabilGuard® (a BSA-free blocking agent; Product code SG02, Surmodics, Eden Prairie, Minn.), chaotropic agents such as guanidium hydro chloride, and detergents or surfactants. Useful ionic detergents include sodium dodecyl sulfate (SDS, sodium lauryl sulfate (SLS)), sodium laureth sulfate (SLS, sodium lauryl ether sulfate (SLES)), ammonium lauryl sulfate (ALS), cetrimonium bromide, cetrimonium chloride, cetrimonium stearate, and the like. Useful non-ionic (zwitterionic) detergents include polyoxyethylene glycols, polysorbate 20 (also known as Tween 20), other polysorbates (e.g., 40, 60, 65, 80, etc), Triton-X (e.g., X100, X114), 3-[(3-cholamidopropyl)dimethylammonio]-1-propanesulfonate (CHAPS), CHAPSO, deoxycholic acid, sodium deoxycholate, NP-40, glycosides, octyl-thio-glucosides, maltosides, and the like. In some embodiments, Pluronic F-68, a surfactant shown to reduce platelet aggregation, is used to treat samples containing vesicles during isolation and/or detection. F68 can be used from a 0.1% to 10% concentration, e.g., a 1%, 2.5% or 5% concentration. The pH and/or ionic strength of the solution can be adjusted with various acids, bases, buffers or salts, including without limitation sodium chloride (NaCl), phosphate-buffered saline (PBS), tris-buffered saline (TBS), sodium phosphate, potassium chloride, potassium phosphate, sodium citrate and saline-sodium citrate (SSC) buffer. In some embodiments, NaCl is added at a concentration of 0.1% to 10%, e.g., 1%, 2.5% or 5% final concentration. In some embodiments, Tween 20 is added to 0.005 to 2% concentration, e.g., 0.05%, 0.25% or 0.5% final concentration. Blocking agents for use with the invention comprise inert proteins, e.g., milk proteins, non-fat dry milk protein, albumin, BSA, casein, or serum such as newborn calf serum (NBCS), goat serum, rabbit serum or salmon serum. The proteins can be added at a 0.1% to 10% concentration, e.g., 1%, 2%, 3%, 3.5%, 4%, 5%, 6%, 7%, 8%, 9% or 10% concentration. In some embodiments, BSA is added to 0.1% to 10% concentration, e.g., 1%, 2%, 3%, 3.5%, 4%, 5%, 6%, 7%, 8%, 9% or 10% concentration. In some embodiments, the sample is treated according to the methodology presented in U.S. patent application Ser. No. 11/632,946, filed Jul. 13, 2005, which application is incorporated herein by reference in its entirety. Commercially available blockers may be used, such as SuperBlock, StartingBlock, Protein-Free from Pierce (a division of Thermo Fisher Scientific, Rockford, Ill.). In some embodiments, SSC/detergent (e.g., 20×SSC with 0.5% Tween 20 or 0.1% Triton-X 100) is added to 0.1% to 10% concentration, e.g., at 1.0% or 5.0% concentration.

The methods of detecting vesicles and other circulating biomarkers can be optimized as desired with various combinations of protocols and treatments as described herein. A detection protocol can be optimized by various combinations of agitation, isolation methods, and additives. In some embodiments, the patient sample is vortexed before and after isolation steps, and the sample is treated with blocking agents including BSA and/or F68. Such treatments may reduce the formation of large aggregates or protein or other biological debris and thus provide a more consistent detection reading.

Filtration and Ultrafiltration

A vesicle can be isolated from a biological sample by filtering a biological sample from a subject through a filtration module and collecting from the filtration module a retentate comprising the vesicle, thereby isolating the vesicle from the biological sample. The method can comprise filtering a biological sample from a subject through a filtration module comprising a filter (also referred to herein as a selection membrane); and collecting from the filtration module a retentate comprising the vesicle, thereby isolating the vesicle from the biological sample. For example, in some embodiments, the filter retains molecules greater than about 100 kiloDaltons. In such cases, microvesicles are generally found within the retentate of the filtration process whereas smaller entities such as proteins, protein complexes, nucleic acids, etc, pass through into the filtrate.

The method can be used when determining a biosignature of one or more microvesicle. The method can also further comprise contacting the retentate from the filtration to a plurality of substrates, wherein each substrate is coupled to one or more capture agents, and each subset of the plurality of substrates comprises a different capture agent or combination of capture agents than another subset of the plurality of substrates.

Also provided herein is a method of determining a biosignature of a vesicle in a sample comprising: filtering a biological sample from a subject with a disorder through a filtration module, collecting from the filtration module a retentate comprising one or more vesicles, and determining a biosignature of the one or more vesicles. In some embodiments, the filtration module comprises a filter that retains molecules greater than about 100 or 150 kiloDaltons.

The method disclosed herein can further comprise characterizing a phenotype in a subject by filtering a biological sample from a subject through a filtration module, collecting from the filtration module a retentate comprising one or more vesicles; detecting a biosignature of the one or more vesicles; and characterizing a phenotype in the subject based on the biosignature, wherein characterizing is with at least 70% sensitivity. In some embodiments, characterizing comprises determining an amount of one or more vesicle having the biosignature. Furthermore, the characterizing can be from about 80% to 100% sensitivity.

Also provided herein is a method for multiplex analysis of a plurality of vesicles. In some embodiments, the method comprises filtering a biological sample from a subject through a filtration module; collecting from the filtration module a retentate comprising the plurality of vesicles, applying the plurality of vesicles to a plurality of capture agents, wherein the plurality of capture agents is coupled to a plurality of substrates, and each subset of the plurality of substrates is differentially labeled from another subset of the plurality of substrates; capturing at least a subset of the plurality of vesicles; and determining a biosignature for at least a subset of the captured vesicles. In some embodiments, each substrate is coupled to one or more capture agents, and each subset of the plurality of substrates comprises a different capture agent or combination of capture agents as compared to another subset of the plurality of substrates. In some embodiments, at least a subset of the plurality of substrates is intrinsically labeled, such as comprising one or more labels. The substrate can be a particle or bead, or any combination thereof. In some embodiments, the filter retains molecules greater than 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, or greater than 10000 kiloDaltons (kDa). In some embodiments, the filtration module comprises a filter that retains molecules greater than about 100 or 150 kiloDaltons. In some embodiments, the filtration module comprises a filter that retains molecules greater than about 9, 20, 100 or 150 kiloDaltons. In still other embodiments, the filtration module comprises a filter that retains molecules greater than about 7000 kDa.

In some embodiments, the method for multiplex analysis of a plurality of vesicles comprises filtering a biological sample from a subject through a filtration module, wherein the filtration module comprises a filter that retains molecules greater than about 100 kiloDaltons; collecting from the filtration module a retentate comprising the plurality of vesicles; applying the plurality of vesicles to a plurality of capture agents, wherein the plurality of capture agents is coupled to a microarray; capturing at least a subset of the plurality of vesicles on the microarray; and determining a biosignature for at least a subset of the captured vesicles. In some embodiments, the filter retains molecules greater than 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, or greater than 10000 kiloDaltons (kDa). In some embodiments, the filtration module comprises a filter that retains molecules greater than about 100 or 150 kiloDaltons. In some embodiments, the filtration module comprises a filter that retains molecules greater than about 9, 20, 100 or 150 kiloDaltons. In still other embodiments, the filtration module comprises a filter that retains molecules greater than about 7000 kDa.

The biological sample can be clarified prior to isolation by filtration. Clarification comprises selective removal of cellular debris and other undesirable materials. For example, cellular debris and other components that may interfere with detection of the circulating biomarkers can be removed. The clarification can be by low-speed centrifugation, such as at about 5,000×g, 4,000×g, 3,000×g, 2,000×g, 1,000×g, or less. The supernatant, or clarified biological sample, containing the vesicle can then be collected and filtered to isolate the vesicle from the clarified biological sample. In some embodiments, the biological sample is not clarified prior to isolation of a vesicle by filtration.

In some embodiments, isolation of a vesicle from a sample does not use high-speed centrifugation, such as ultracentrifugation. For example, isolation may not require the use of centrifugal speeds, such as about 100,000×g or more. In some embodiments, isolation of a vesicle from a sample uses speeds of less than 50,000×g, 40,000×g, 30,000×g, 20,000×g, 15,000×g, 12,000×g, or 10,000×g.

Any number of applicable filter configurations can be used to filter a sample of interest. In some embodiments, the filtration module used to isolate the circulating biomarkers from the biological sample is a fiber-based filtration cartridge. For example, the fiber can be a hollow polymeric fiber, such as a polypropylene hollow fiber. A biological sample can be introduced into the filtration module by pumping the sample fluid, such as a biological fluid as disclosed herein, into the module with a pump device, such as a peristaltic pump. The pump flow rate can vary, such as at about 0.25, 0.5, 1, 1.5, 2, 2.5, 3, 3.5, 4, 4.5, 5, 6, 7, 8, 9, or 10 mL/minute. The flow rate can be adjusted given the configuration, e.g., size and throughput, of the filtration module.

The filtration module can be a membrane filtration module. For example, the membrane filtration module can comprise a filter disc membrane, such as a hydrophilic polyvinylidene difluoride (PVDF) filter disc membrane housed in a stirred cell apparatus (e.g., comprising a magnetic stirrer). In some embodiments, the sample moves through the filter as a result of a pressure gradient established on either side of the filter membrane.

The filter can comprise a material having low hydrophobic absorptivity and/or high hydrophilic properties. For example, the filter can have an average pore size for vesicle retention and permeation of most proteins as well as a surface that is hydrophilic, thereby limiting protein adsorption. For example, the filter can comprise a material selected from the group consisting of polypropylene, PVDF, polyethylene, polyfluoroethylene, cellulose, secondary cellulose acetate, polyvinylalcohol, and ethylenevinyl alcohol (EVAL®, Kuraray Co., Okayama, Japan). Additional materials that can be used in a filter include, but are not limited to, polysulfone and polyethersulfone.

The filtration module can have a filter that retains molecules greater than about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, or 900 kiloDaltons (kDa), such as a filter that has a MWCO (molecular weight cut off) of about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, or 900 kDa, respectively. In embodiments, the filtration module has a MWCO of 1000 kDa, 1500 kDa, 2000 kDa, 2500 kDa, 3000 kDa, 3500 kDa, 4000 kDa, 4500 kDa, 5000 kDa, 5500 kDa, 6000 kDa, 6500 kDa, 7000 kDa, 7500 kDa, 8000 kDa, 8500 kDa, 9000 kDa, 9500 kDa, 10000 kDa, or greater than 10000 kDa. Ultrafiltration membranes with a range of MWCO of 9 kDa, 20 kDa and/or 150 kDa can be used. In some embodiments, the filter within the filtration module has an average pore diameter of about 0.01 μm to about 0.15 μm, and in some embodiments from about 0.05 μm to about 0.12 μm. In some embodiments, the filter has an average pore diameter of about 0.06 μm, 0.07 μm, 0.08 μm, 0.09 μm, 0.1 μm, 0.11 μm or 0.2 μm.

The filtration module can be a commerically available column, such as a column typically used for concentrating proteins or for isolating proteins (e.g., ultrafiltration). Examples include, but are not limited to, columns from Millpore (Billerica, Mass.), such as Amicon® centrifugal filters, or from Pierce® (Rockford, Ill.), such as Pierce Concentrator filter devices. Useful columns from Pierce include disposable ultrafiltration centrifugal devices with a MWCO of 9 kDa, 20 kDa and/or 150 kDa. These concentrators consist of a high-performance regenerated cellulose membrane welded to a conical device. The filters can be as described in U.S. Pat. No. 6,269,957 or 6,357,601, both of which applications are incorporated by reference in their entirety herein.

The retentate comprising the isolated vesicle can be collected from the filtration module. The retentate can be collected by flushing the retentate from the filter. Selection of a filter composition having hydrophilic surface properties, thereby limiting protein adsorption, can be used, without being bound by theory, for easier collection of the retentate and minimize use of harsh or time-consuming collection techniques.

The collected retentate can then be used subsequent analysis, such as assessing a biosignature of one or more vesicles in the retentate, as further described herein. The analysis can be directly performed on the collected retentate. Alternatively, the collected retentate can be further concentrated or purified, prior to analysis of one or more vesicles. For example, the retentate can be further concentrated or vesicles further isolated from the retentate using size exclusion chromatography, density gradient centrifugation, differential centrifugation, immunoabsorbent capture, affinity purification, microfluidic separation, or combinations thereof, such as described herein. In some embodiments, the retentate can undergo another step of filtration. Alternatively, prior to isolation of a vesicle using a filter, the vesicle is concentrated or isolated using techniques including without limitation size exclusion chromatography, density gradient centrifugation, differential centrifugation, immunoabsorbent capture, affinity purification, microfluidic separation, or combinations thereof

Combinations of filters can be used for concentrating and isolating biomarkers. For example, the biological sample may first be filtered through a filter having a porosity or pore size of between about 0.01 μm to about 10 μm, e.g., 0.01 μm to about 2 μm or about 0.05 μm to about 1.5 μm, and then the sample is filtered. For example, prior to filtering a biological sample through a filtration module with a filter that retains molecules greater than about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, or greater than 10000 kiloDaltons (kDa), such as a filter that has a MWCO (molecular weight cut off) of about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, or greater than 10000 kDa, respectively, the biological sample may first be filtered through a filter having a porosity or pore size of between about 0.01 μm to about 10 μm, e.g., 0.01 μm to about 2 μm or about 0.05 μm to about 1.5 μm. In some embodiments, the filter has a pore size of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0, 3.0, 4.0, 5.0, 6.0, 7.0, 8.0, 9.0 or 10.0 μm. The filter may be a syringe filter. Thus, in some embodiments, the method comprises filtering the biological sample through a filter, such as a syringe filter, wherein the syringe filter has a porosity of greater than about 1 μm, prior to filtering the sample through a filtration module comprising a filter that retains molecules greater than about 100 or 150 kiloDaltons. In some embodiments, the filter is 1.2 μM filter and the filtration is followed by passage of the sample through a 7 ml or 20 ml concentrator column with a 150 kDa cutoff. Multiple concentrator columns may be used, e.g., in series. For example, a 7000 MWCO filtration unit can be used before a 150 MWCO unit.

The filtration module can be a component of a microfluidic device. Microfluidic devices, which may also be referred to as “lab-on-a-chip” systems, biomedical micro-electro-mechanical systems (bioMEMs), or multicomponent integrated systems, can be used for isolating, and analyzing, vesicles. Such systems miniaturize and compartmentalize processes that allow for binding of vesicles, detection of biomarkers, and other processes, such as further described herein.

The filtration module and assessment can be as described in Grant, R., et al., A filtration-based protocol to isolate human Plasma Membrane-derived Vesicles and exosomes from blood plasma, J Immunol Methods (2011) 371:143-51 (Epub 2011 Jun. 30), which reference is incorporated herein by reference in its entirety.

A microfluidic device can also be used for isolation of a vesicle by comprising a filtration module. For example, a microfluidic device can use one more channels for isolating a vesicle from a biological sample based on size from a biological sample. A biological sample can be introduced into one or more microfluidic channels, which selectively allows the passage of vesicles. The microfluidic device can further comprise binding agents, or more than one filtration module to select vesicles based on a property of the vesicles, for example, size, shape, deformability, biomarker profile, or biosignature.

The retentate from a filtration step can be further processed before assessment of microvesicles or other biomarkers therein. In some embodiments, the retentate is diluted prior to biomarker assessment, e.g., with an appropriate diluent such as a biologically compatible buffer. In some cases, the retentate is serially diluted. In an aspect, the invention provides a method for detecting a microvesicle population from a biological sample comprising: a) concentrating the biological sample using a selection membrane having a pore size of from 0.01 μm to about 10 μm, or a molecular weight cut off (MWCO) from about 1 kDa to 10000 kDa; b) diluting a retentate from the concentration step into one or more aliquots; and c) contacting each of the one or more aliquots of retentate with one or more binding agent specific to a molecule of at least one microvesicle in the microvesicle population. In a related aspect, the invention provides a method for detecting a microvesicle population from a biological sample comprising: a) concentrating the biological sample using a selection membrane having a pore size of from 0.01 μm to about 10 μm, or a molecular weight cut off (MWCO) from about 1 kDa to 10000 kDa; and b) contacting one or more aliquots of the retentate from the concentrating step with one or more binding agent specific to a molecule of at least one microvesicle in the microvesicle population.

The selection membrane can be sized to retain the desired biomarkers in the retentate or to allow the desired biomarkers to pass through the filter into the filtrate. The filter membrane can be chosen to have a certain pore size or MWCO value. The selection membrane can have a pore size of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0, 3.0, 4.0, 5.0, 6.0, 7.0, 8.0, 9.0 or 10.0 μm. The selection membrane can also have a MWCO of about 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 2000, 3000, 4000, 5000, 6000, 7000, 8000, 9000 or 10000 kDa.

The retentate can be separated and/or diluted into any number of desired aliquots. For example, multiple aliquots without any dilution or the same dilution can be used to determine reproducibility. In another example, multiple aliquots at different dilutions can be used to construct a concentration curve. In some embodiments, the retentate is separated and/or diluted into at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350 or 400 aliquots. The aliquots can be at a same dilution or at different dilutions.

A dilution factor is the ratio of the final volume of a mixture (the mixture of the diluents and aliquot) divided by the initial volume of the aliquot. The retentate can be diluted into one or more aliquots at a dilution factor of about 0, 1, 2, 3, 4, 5, 6, 7, 8, 9, 10, 15, 20, 25, 30, 40, 50, 60, 70, 80, 90, 100, 110, 120, 130, 140, 150, 160, 170, 180, 190, 200, 250, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 20000, 30000, 40000, 50000, 60000, 70000, 80000, 90000 and/or 100000. For example, the retentate can be diluted into one or more aliquot at a dilution factor of about 500.

To estimate a concentration or form a curve, the retentate can be diluted into multiple aliquots. In some embodiments of the method, the retentate is diluted into one or more aliquots at a dilution factor of about 100, 250, 500, 1000, 10000 and 100000. As desired, the method can further comprise detecting an amount of microvesicles in each aliquot of retentate, e.g., that formed a complex with the one or more binding agent. The curve can be used to determine a linear range of the amount of microvesicles in each aliquot detected versus dilution factor. A concentration of the detected microvesicles for the biological sample can be determined using the amount of microvesicles determined in one or more aliquot within the linear range. The concentration can be compared to a reference concentration, e.g., in order to characterize a phenotype as described herein.

The invention also provides a related method comprising filtering a biological sample from a subject through a filtration module and collecting a filtrate comprising the vesicle, thereby isolating the vesicle from the biological sample. In such cases cells and other large entities can be retained in the retentate while microvesicles pass through into the filtrate. It will be appreciated that strategies to retain and filter microvesicles can be used in concert. For example, a sample can be filtered with a selection membrane that allows microvesicles to pass through, thereby isolating the microvesicles from large particles (cells, complexes, etc). The filtrate comprising the microvesicle can then be filtered using a selection membrane that retains microvesicles, thereby isolating the microvesicles from smaller particles (proteins, nucleic acids, etc). The isolated microvesicles can be further assessed according to the methods of the invention, e.g., to characterize a phenotype.

Precipitation

Vesicles can be isolated using a polymeric precipitation method. The method can be in combination with or in place of the other isolation methods described herein. In some embodiments, the sample containing the vesicles is contacted with a formulation of polyethylene glycol (PEG). The polymeric formulation is incubated with the vesicle containing sample then precipitated by centrifugation. The PEG can bind to the vesicles and can be treated to specifically capture vesicles by addition of a capture moiety, e.g., a pegylated-binding protein such as an antibody. One of skill will appreciate that other polymers in addition to PEG can be used, e.g., PEG derivatives including methoxypolyethylene glycols, poly (ethylene oxide), and various polymers of formula HO—CH₂—(CH₂—O—CH₂-)n-CH₂—OH having different molecular weights. The efficiency of isolation may depend on various factors including the length of the polymer chains and concentration of polymer used. In preferred embodiments, PEG4000 or PEG 8000 may be used at a concentration of 1%, 2%, 3%, 4%, 5%, 6%, 7%, 8%, 9%, or 10%, e.g., 4% or 8%.

In some embodiments of the invention, the vesicles are concentrated from a sample using the polymer precipitation method and the isolated vesicles are further separated using another approach. The second step can be used to identify a subpopulation of vesicles, e.g., that display certain biomarkers. The second separation step can comprise size exclusion, a binding agent, an antibody capture step, microbeads, as described herein.

In some embodiments, vesicles are isolated according to the ExoQuick™ and ExoQuick-TC™ kits from System Biosciences, Mountain View, Calif. USA. These kits use a polymer-based precipitation method to pellet vesicles. Similarly, the vesicles can be isolated using the Total Exosome Isolation (from Serum) or Total Exosome Isolation (from Cell Culture Media) kits from Invitrogen/Life Technologies (Carlsbad, Calif. USA). The Total Exosome Isolation reagent forces less-soluble components such as vesicles out of solution, allowing them to be collected by a short, low-speed centrifugation. The reagent is added to the biological sample, and the solution is incubated overnight at 2° C. to 8° C. The precipitated vesicles are recovered by standard centrifugation.

Binding Agents

Binding agents (also referred to as binding reagents) include agents that are capable of binding a target biomarker. A binding agent can be specific for the target biomarker, meaning the agent is capable of binding a target biomarker. The target can be any useful biomarker disclosed herein, such as a biomarker on the vesicle surface. In some embodiments, the target is a single molecule, such as a single protein, so that the binding agent is specific to the single protein. In other embodiments, the target can be a group of molecules, such as a family or proteins having a similar epitope or moiety, so that the binding agent is specific to the family or group of proteins. The group of molecules can also be a class of molecules, such as protein, DNA or RNA. The binding agent can be a capture agent used to capture a vesicle by binding a component or biomarker of a vesicle. In some embodiments, a capture agent comprises an antibody or an aptamer that binds to an antigen on a vesicle. The capture agent can be optionally coupled to a substrate and used to isolate a vesicle, as further described herein.

A binding agent is an agent that binds to a circulating biomarker, such as a vesicle or a component of a vesicle. The binding agent can be used as a capture agent and/or a detection agent. A capture agent can bind and capture a circulating biomarker, such as by binding a component or biomarker of a vesicle. For example, the capture agent can be a capture antibody or capture antigen that binds to an antigen on a vesicle. A detection agent can bind to a circulating biomarker thereby facilitating detection of the biomarker. For example, a capture agent comprising an antibody or aptamer that is sequestered to a substrate can be used to capture a vesicle in a sample, and a detection agent comprising an antibody or aptamer that carries a label can be used to detect the captured vesicle via detection of the detection agent's label. In some embodiments, a vesicle is assessed using capture and detection agents that recognize the same vesicle biomarkers. For example, a vesicle population can be captured using a tetraspanin such as by using an anti-CD9 antibody bound to a substrate, and the captured vesicles can be detected using a fluorescently labeled anti-CD9 antibody to label the captured vesicles. In other embodiments, a vesicle is assessed using capture and detection agents that recognize different vesicle biomarkers. For example, a vesicle population can be captured using a cell-specific marker such as by using an anti-PCSA antibody bound to a substrate, and the captured vesicles can be detected using a fluorescently labeled anti-CD9 antibody to label the captured vesicles. Similarly, the vesicle population can be captured using a general vesicle marker such as by using an anti-CD9 antibody bound to a substrate, and the captured vesicles can be detected using a fluorescently labeled antibody to a cell-specific or disease specific marker to label the captured vesicles.

The biomarkers recognized by the binding agent are sometimes referred to herein as an antigen. Unless otherwise specified, antigen as used herein is meant to encompass any entity that is capable of being bound by a binding agent, regardless of the type of binding agent or the immunogenicity of the biomarker. The antigen further encompasses a functional fragment thereof. For example, an antigen can encompass a protein biomarker capable of being bound by a binding agent, including a fragment of the protein that is capable of being bound by a binding agent.

In some embodiments, a vesicle is captured using a capture agent that binds to a biomarker on a vesicle. The capture agent can be coupled to a substrate and used to isolate a vesicle, as further described herein. In some embodiments, a capture agent is used for affinity capture or isolation of a vesicle present in a substance or sample.

A binding agent can be used after a vesicle is concentrated or isolated from a biological sample. For example, a vesicle can first be isolated from a biological sample before a vesicle with a specific biosignature is isolated or detected. The vesicle with a specific biosignature can be isolated or detected using a binding agent for the biomarker. A vesicle with the specific biomarker can be isolated or detected from a heterogeneous population of vesicles. Alternatively, a binding agent may be used on a biological sample comprising vesicles without a prior isolation or concentration step. For example, a binding agent is used to isolate or detect a vesicle with a specific biosignature directly from a biological sample.

A binding agent can be a nucleic acid, protein, or other molecule that can bind to a component of a vesicle. The binding agent can comprise DNA, RNA, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), lectins, synthetic or naturally occurring chemical compounds (including but not limited to drugs, labeling reagents), dendrimers, or a combination thereof. For example, the binding agent can be a capture antibody. In embodiments of the invention, the binding agent comprises a membrane protein labeling agent. See, e.g., the membrane protein labeling agents disclosed in Alroy et al., US. Patent Publication US 2005/0158708. In some embodiments, vesicles are isolated or captured as described herein, and one or more membrane protein labeling agent is used to detect the vesicles.

In some instances, a single binding agent can be employed to isolate or detect a vesicle. In other instances, a combination of different binding agents may be employed to isolate or detect a vesicle. For example, at least 2, 3, 4, 5, 6, 7, 8, 9, 10, 11, 12, 13, 14, 15, 16, 17, 18, 19, 20, 25, 50, 75 or 100 different binding agents may be used to isolate or detect a vesicle from a biological sample. Furthermore, the one or more different binding agents for a vesicle can form a biosignature of a vesicle, as further described below.

Different binding agents can also be used for multiplexing. For example, isolation or detection of more than one population of vesicles can be performed by isolating or detecting each vesicle population with a different binding agent. Different binding agents can be bound to different particles, wherein the different particles are labeled. In some embodiments, an array comprising different binding agents can be used for multiplex analysis, wherein the different binding agents are differentially labeled or can be ascertained based on the location of the binding agent on the array. Multiplexing can be accomplished up to the resolution capability of the labels or detection method, such as described below. The binding agents can be used to detect the vesicles, such as for detecting cell-of-origin specific vesicles. A binding agent or multiple binding agents can themselves form a binding agent profile that provides a biosignature for a vesicle. One or more binding agents can be selected from FIG. 2 of International Patent Publication No. WO/2011/127219, entitled “Circulating Biomarkers for Disease” and filed Apr. 6, 2011, which application is incorporated by reference in its entirety herein. For example, if a vesicle population is detected or isolated using two, three, four or more binding agents in a differential detection or isolation of a vesicle from a heterogeneous population of vesicles, the particular binding agent profile for the vesicle population provides a biosignature for the particular vesicle population. The vesicle can be detected using any number of binding agents in a multiplex fashion. Thus, the binding agent can also be used to form a biosignature for a vesicle. The biosignature can be used to characterize a phenotype.

The binding agent can be a lectin. Lectins are proteins that bind selectively to polysaccharides and glycoproteins and are widely distributed in plants and animals. For example, lectins such as those derived from Galanthus nivalis in the form of Galanthus nivalis agglutinin (“GNA”), Narcissus pseudonarcissus in the form of Narcissus pseudonarcissus agglutinin (“NPA”) and the blue green algae Nostoc ellipsosporum called “cyanovirin” (Boyd et al. Antimicrob Agents Chemother 41(7): 1521 1530, 1997; Hammar et al. Ann N Y Acad Sci 724: 166169, 1994; Kaku et al. Arch Biochem Biophys 279(2): 298 304, 1990) can be used to isolate a vesicle. These lectins can bind to glycoproteins having a high mannose content (Chervenak et al. Biochemistry 34(16): 5685 5695, 1995). High mannose glycoprotein refers to glycoproteins having mannose-mannose linkages in the form of α-1→3 or α-1→6 mannose-mannose linkages.

The binding agent can be an agent that binds one or more lectins. Lectin capture can be applied to the isolation of the biomarker cathepsin D since it is a glycosylated protein capable of binding the lectins Galanthus nivalis agglutinin (GNA) and concanavalin A (ConA).

Methods and devices for using lectins to capture vesicles are described in International Patent Publications WO/2011/066589, entitled “METHODS AND SYSTEMS FOR ISOLATING, STORING, AND ANALYZING VESICLES” and filed Nov. 30, 2010; WO/2010/065765, entitled “AFFINITY CAPTURE OF CIRCULATING BIOMARKERS” and filed Dec. 3, 2009; WO/2010/141862, entitled “METHODS AND MATERIALS FOR ISOLATING EXOSOMES” and filed Jun. 4, 2010; and WO/2007/103572, entitled “EXTRACORPOREAL REMOVAL OF MICROVESICULAR PARTICLES” and filed Mar. 9, 2007, each of which applications is incorporated by reference herein in its entirety.

The binding agent can be an antibody. For example, a vesicle may be isolated using one or more antibodies specific for one or more antigens present on the vesicle. For example, a vesicle can have CD63 on its surface, and an antibody, or capture antibody, for CD63 can be used to isolate the vesicle. Alternatively, a vesicle derived from a tumor cell can express EpCam, the vesicle can be isolated using an antibody for EpCam and CD63. Other antibodies for isolating vesicles can include an antibody, or capture antibody, to CD9, PSCA, TNFR, CD63, B7H3, MFG-E8, EpCam, Rab, CD81, STEAP, PCSA, PSMA, or 5T4. Other antibodies for isolating vesicles can include an antibody, or capture antibody, to DR3, STEAP, epha2, TMEM211, MFG-E8, Tissue Factor (TF), unc93A, A33, CD24, NGAL, EpCam, MUC17, TROP2, or TETS.

In some embodiments, the capture agent is an antibody to CD9, CD63, CD81, PSMA, PCSA, B7H3, EpCam, PSCA, ICAM, STEAP, or EGFR. The capture agent can also be used to identify a biomarker of a vesicle. For example, a capture agent such as an antibody to CD9 would identify CD9 as a biomarker of the vesicle. In some embodiments, a plurality of capture agents can be used, such as in multiplex analysis. The plurality of captures agents can comprise binding agents to one or more of: CD9, CD63, CD81, PSMA, PCSA, B7H3, EpCam, PSCA, ICAM, STEAP, and EGFR. In some embodiments, the plurality of capture agents comprise binding agents to CD9, CD63, CD81, PSMA, PCSA, B7H3, MFG-E8, and/or EpCam. In yet other embodiments, the plurality of capture agents comprises binding agents to CD9, CD63, CD81, PSMA, PCSA, B7H3, EpCam, PSCA, ICAM, STEAP, and/or EGFR. The plurality of capture agents comprises binding agents to TMEM211, MFG-E8, Tissue Factor (TF), and/or CD24.

The antibodies referenced herein can be immunoglobulin molecules or immunologically active portions of immunoglobulin molecules, i.e., molecules that contain an antigen binding site that specifically binds an antigen and synthetic antibodies. The immunoglobulin molecules can be of any class (e.g., IgG, IgE, IgM, IgD or IgA) or subclass of immunoglobulin molecule. Antibodies include, but are not limited to, polyclonal, monoclonal, bispecific, synthetic, humanized and chimeric antibodies, single chain antibodies, Fab fragments and F(ab′)₂ fragments, Fv or Fv′ portions, fragments produced by a Fab expression library, anti-idiotypic (anti-Id) antibodies, or epitope-binding fragments of any of the above. An antibody, or generally any molecule, “binds specifically” to an antigen (or other molecule) if the antibody binds preferentially to the antigen, and, e.g., has less than about 30%, 20%, 10%, 5% or 1% cross-reactivity with another molecule.

The binding agent can also be a polypeptide or peptide. Polypeptide is used in its broadest sense and may include a sequence of subunit amino acids, amino acid analogs, or peptidomimetics. The subunits may be linked by peptide bonds. The polypeptides may be naturally occurring, processed forms of naturally occurring polypeptides (such as by enzymatic digestion), chemically synthesized or recombinantly expressed. The polypeptides for use in the methods of the present invention may be chemically synthesized using standard techniques. The polypeptides may comprise D-amino acids (which are resistant to L-amino acid-specific proteases), a combination of D- and L-amino acids, β amino acids, or various other designer or non-naturally occurring amino acids (e.g., β-methyl amino acids, Cα-methyl amino acids, and Na-methyl amino acids, etc.) to convey special properties. Synthetic amino acids may include ornithine for lysine, and norleucine for leucine or isoleucine. In addition, the polypeptides can have peptidomimetic bonds, such as ester bonds, to prepare polypeptides with novel properties. For example, a polypeptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo. Polypeptides can also include peptoids (N-substituted glycines), in which the side chains are appended to nitrogen atoms along the molecule's backbone, rather than to the a-carbons, as in amino acids. Polypeptides and peptides are intended to be used interchangeably throughout this application, i.e. where the term peptide is used, it may also include polypeptides and where the term polypeptides is used, it may also include peptides. The term “protein” is also intended to be used interchangeably throughout this application with the terms “polypeptides” and “peptides” unless otherwise specified.

A vesicle may be isolated, captured or detected using a binding agent. The binding agent can be an agent that binds a vesicle “housekeeping protein,” or general vesicle biomarker. The biomarker can be CD63, CD9, CD81, CD82, CD37, CD53, Rab-5b, Annexin V, MFG-E8 or other commonly observed vesicle markers include those listed in Table 3. Furthermore, any of the markers disclosed herein or in Table 3 can be selected in identifying a candidate biosignature for a disease or condition, where the one or more selected biomarkers have a direct or indirect role or function in mechanisms involved in the disease or condition.

The binding agent can also be an agent that binds to a vesicle derived from a specific cell type, such as a tumor cell (e.g. binding agent for Tissue factor, EpCam, B7H3, RAGE or CD24) or a specific cell-of-origin. The binding agent used to isolate or detect a vesicle can be a binding agent for an antigen selected from FIG. 1 of International Patent Publication No. WO/2011/127219, entitled “Circulating Biomarkers for Disease” and filed Apr. 6, 2011, which application is incorporated by reference in its entirety herein. The binding agent for a vesicle can also be selected from those listed in FIG. 2 of International Patent Publication No. WO/2011/127219. The binding agent can be for an antigen such as a tetraspanin, MFG-E8, Annexin V, 5T4, B7H3, caveolin, CD63, CD9, E-Cadherin, Tissue factor, MFG-E8, TMEM211, CD24, PSCA, PCSA, PSMA, Rab-5B, STEAP, TNFR1, CD81, EpCam, CD59, CD81, ICAM, EGFR, or CD66. A binding agent for a platelet can be a glycoprotein such as GpIa-IIa, GpIIb-IIIa, GpIIIb, GpIb, or GpIX. A binding agent can be for an antigen comprising one or more of CD9, Erb2, Erb4, CD81, Erb3, MUC16, CD63, DLL4, HLA-Drpe, B7H3, IFNAR, 5T4, PCSA, MICB, PSMA, MFG-E8, Muc1, PSA, Muc2, Unc93a, VEGFR2, EpCAM, VEGF A, TMPRSS2, RAGE, PSCA, CD40, Muc17, IL-17-RA, and CD80. For example, the binding agent can be one or more of CD9, CD63, CD81, B7H3, PCSA, MFG-E8, MUC2, EpCam, RAGE and Muc17. One or more binding agents, such as one or more binding agents for two or more of the antigens, can be used for isolating or detecting a vesicle. The binding agent used can be selected based on the desire of isolating or detecting a vesicle derived from a particular cell type or cell-of-origin specific vesicle. The binding agent can be to one or more vesicle marker in Table 6 as desired.

A binding agent can also be linked directly or indirectly to a solid surface or substrate. A solid surface or substrate can be any physically separable solid to which a binding agent can be directly or indirectly attached including, but not limited to, surfaces provided by microarrays and wells, particles such as beads, columns, optical fibers, wipes, glass and modified or functionalized glass, quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polypropylene, polyethylene, polybutylene, polyurethanes, polytetrafluoroethylene (PTFE, Teflon®), etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In addition, as is known the art, the substrate may be coated using passive or chemically-derivatized coatings with any number of materials, including polymers, such as dextrans, acrylamides, gelatins or agarose. Such coatings can facilitate the use of the array with a biological sample.

For example, an antibody used to isolate a vesicle can be bound to a solid substrate such as a well, such as commercially available plates (e.g. from Nunc, Milan Italy). Each well can be coated with the antibody. In some embodiments, the antibody used to isolate a vesicle is bound to a solid substrate such as an array. The array can have a predetermined spatial arrangement of molecule interactions, binding islands, biomolecules, zones, domains or spatial arrangements of binding islands or binding agents deposited within discrete boundaries. Further, the term array may be used herein to refer to multiple arrays arranged on a surface, such as would be the case where a surface bore multiple copies of an array. Such surfaces bearing multiple arrays may also be referred to as multiple arrays or repeating arrays.

Arrays typically contain addressable moieties that can detect the presense of an entity, e.g., a vesicle in the sample via a binding event. An array may be referred to as a microarray. Arrays or microarrays include without limitation DNA microarrays, such as cDNA microarrays, oligonucleotide microarrays and SNP microarrays, microRNA arrays, protein microarrays, antibody microarrays, tissue microarrays, cellular microarrays (also called transfection microarrays), chemical compound microarrays, and carbohydrate arrays (glycoarrays). DNA arrays typically comprise addressable nucleotide sequences that can bind to sequences present in a sample. MicroRNA arrays, e.g., the MMChips array from the University of Louisville or commercial systems from Agilent, can be used to detect microRNAs. Protein microarrays can be used to identify protein—protein interactions, including without limitation identifying substrates of protein kinases, transcription factor protein-activation, or to identify the targets of biologically active small molecules. Protein arrays may comprise an array of different protein molecules, commonly antibodies, or nucleotide sequences that bind to proteins of interest. In a non-limiting example, a protein array can be used to detect vesicles having certain proteins on their surface. Antibody arrays comprise antibodies spotted onto the protein chip that are used as capture molecules to detect proteins or other biological materials from a sample, e.g., from cell or tissue lysate solutions. For example, antibody arrays can be used to detect vesicle-associated biomarkers from bodily fluids, e.g., serum or urine. Tissue microarrays comprise separate tissue cores assembled in array fashion to allow multiplex histological analysis. Cellular microarrays, also called transfection microarrays, comprise various capture agents, such as antibodies, proteins, or lipids, which can interact with cells to facilitate their capture on addressable locations. Cellular arrays can also be used to capture vesicles due to the similarity between a vesicle and cellular membrane. Chemical compound microarrays comprise arrays of chemical compounds and can be used to detect protein or other biological materials that bind the compounds. Carbohydrate arrays (glycoarrays) comprise arrays of carbohydrates and can detect, e.g., protein that bind sugar moieties. One of skill will appreciate that similar technologies or improvements can be used according to the methods of the invention.

A binding agent can also be bound to particles such as beads or microspheres. For example, an antibody specific for a component of a vesicle can be bound to a particle, and the antibody-bound particle is used to isolate a vesicle from a biological sample. In some embodiments, the microspheres may be magnetic or fluorescently labeled. In addition, a binding agent for isolating vesicles can be a solid substrate itself. For example, latex beads, such as aldehyde/sulfate beads (Interfacial Dynamics, Portland, Oreg.) can be used.

A binding agent bound to a magnetic bead can also be used to isolate a vesicle. For example, a biological sample such as serum from a patient can be collected for colon cancer screening. The sample can be incubated with anti-CCSA-3 (Colon Cancer-Specific Antigen) coupled to magnetic microbeads. A low-density microcolumn can be placed in the magnetic field of a MACS Separator and the column is then washed with a buffer solution such as Tris-buffered saline. The magnetic immune complexes can then be applied to the column and unbound, non-specific material can be discarded. The CCSA-3 selected vesicle can be recovered by removing the column from the separator and placing it on a collection tube. A buffer can be added to the column and the magnetically labeled vesicle can be released by applying the plunger supplied with the column. The isolated vesicle can be diluted in IgG elution buffer and the complex can then be centrifuged to separate the microbeads from the vesicle. The pelleted isolated cell-of-origin specific vesicle can be resuspended in buffer such as phosphate-buffered saline and quantitated. Alternatively, due to the strong adhesion force between the antibody captured cell-of-origin specific vesicle and the magnetic microbeads, a proteolytic enzyme such as trypsin can be used for the release of captured vesicles without the need for centrifugation. The proteolytic enzyme can be incubated with the antibody captured cell-of-origin specific vesicles for at least a time sufficient to release the vesicles.

A binding agent, such as an antibody, for isolating vesicles is preferably contacted with the biological sample comprising the vesicles of interest for at least a time sufficient for the binding agent to bind to a component of the vesicle. For example, an antibody may be contacted with a biological sample for various intervals ranging from seconds days, including but not limited to, about 10 minutes, 30 minutes, 1 hour, 3 hours, 5 hours, 7 hours, 10 hours, 15 hours, 1 day, 3 days, 7 days or 10 days.

A binding agent, such as an antibody specific to an antigen listed in FIG. 1 of International Patent Publication No. WO/2011/127219, entitled “Circulating Biomarkers for Disease” and filed Apr. 6, 2011, which application is incorporated by reference in its entirety herein, or a binding agent listed in FIG. 2 of International Patent Publication No. WO/2011/127219, can be labeled to facilitate detection. Appropriate labels include without limitation a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles, fluorophores, quantum dots, or radioactive labels. Various protein, radioactive, fluorescent, enzymatic, and other labels are described further above.

A binding agent can be directly or indirectly labeled, e.g., the label is attached to the antibody through biotin-streptavidin. Alternatively, an antibody is not labeled, but is later contacted with a second antibody that is labeled after the first antibody is bound to an antigen of interest.

Depending on the method of isolation or detection used, the binding agent may be linked to a solid surface or substrate, such as arrays, particles, wells and other substrates described above. Methods for direct chemical coupling of antibodies, to the cell surface are known in the art, and may include, for example, coupling using glutaraldehyde or maleimide activated antibodies. Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Biotinylation can be accomplished by, for example, treating the cells with dithiothreitol followed by the addition of biotin maleimide.

Particle-Based Assays

As an alternative to planar arrays, assays using particles or microspheres, such as bead based assays, are capable of use with a binding agent. For example, antibodies or aptamers are easily conjugated with commercially available beads. See, e.g., Fan et al., Illumina universal bead arrays. Methods Enzymol. 2006 410:57-73; Srinivas et al. Anal. Chem. 2011 Oct. 21, Aptamer functionalized Microgel Particles for Protein Detection; See also, review article on aptamers as therapeutic and diagnostic agents, Brody and Gold, Rev. Mol. Biotech. 2000, 74:5-13.

Multiparametric assays or other high throughput detection assays using bead coatings with cognate ligands and reporter molecules with specific activities consistent with high sensitivity automation can be used. In a bead based assay system, a binding agent for a biomarker or vesicle, such as a capture agent (e.g. capture antibody), can be immobilized on an addressable microsphere. Each binding agent for each individual binding assay can be coupled to a distinct type of microsphere (i.e., microbead) and the assay reaction takes place on the surface of the microsphere, such as depicted in FIG. 1B. A binding agent for a vesicle can be a capture antibody or aptamer coupled to a bead. Dyed microspheres with discrete fluorescence intensities are loaded separately with their appropriate binding agent or capture probes. The different bead sets carrying different binding agents can be pooled as desired to generate custom bead arrays. Bead arrays are then incubated with the sample in a single reaction vessel to perform the assay.

Various particle/bead substrates and systems useful for the methods of the invention are described further above.

Flow Cytometry

In various embodiments of the invention, flow cytometry, which is described in further detail above, is used to assess a microvesicle population in a biological sample. If desired, the microvesicle population can be sorted from other particles (e.g., cell debris, protein aggregates, etc) in a sample by labeling the vesicles using one or more general vesicle marker. The general vesicle marker can be a marker in Table 3. Commonly used vesicle markers include tetraspanins such as CD9, CD63 and/or CD81. Vesicles comprising one or more tetraspanin are sometimes refereed to as “Tet+” herein to indicate that the vesicles are tetraspanin-positive. The sorted microvesicles can be further assessed using methodology described herein. E.g., surface antigens on the sorted microvesicles can be detected using flow or other methods. In some embodiments, payload within the sorted microvesicles is assessed. As an illustrative example, a population of microvesicles is contacted with a labeled binding agent to a surface antigen of interest, the contacted microvesicles are sorted using flow cytometry, and payload with the microvesicles is assessed. The payload may be polypeptides, nucleic acids (e.g., mRNA or microRNA) or other biological entities as desired. Such assessment is used to characterize a phenotype as described herein, e.g., to diagnose, prognose or theranose a cancer.

In some embodiments, flow sorting is used to distinguish microvesicle populations from other biological complexes. In a non-limiting example, Ago2+/Tet+ and Ago2+/Tet− particles are detected using flow methodology to separate Ago2+vesicles from vesicle-free Ago2+complexes, respectively.

Multiplexing

Multiplex experiments comprise experiments that can simultaneously measure multiple analytes in a single assay. Vesicles and associated biomarkers can be assessed in a multiplex fashion. Different binding agents can be used for multiplexing different circulating biomarkers, e.g., microRNA, protein, or vesicle populations. Different biomarkers, e.g., different vesicle populations, can be isolated or detected using different binding agents. Each population in a biological sample can be labeled with a different signaling label, such as a fluorophore, quantum dot, or radioactive label, such as described above. The label can be directly conjugated to a binding agent or indirectly used to detect a binding agent that binds a vesicle. The number of populations detected in a multiplexing assay is dependent on the resolution capability of the labels and the summation of signals, as more than two differentially labeled vesicle populations that bind two or more affinity elements can produce summed signals.

Multiplexing of at least 2, 3, 4, 5, 6, 7, 8, 9, 10, 11, 12, 13, 14, 15, 16, 17, 18, 19, 20, 25, 50, 75 or 100 different circulating biomarkers may be performed. For example, one population of vesicles specific to a cell-of-origin can be assayed along with a second population of vesicles specific to a different cell-of-origin, where each population is labeled with a different label. Alternatively, a population of vesicles with a particular biomarker or biosignature can be assayed along with a second population of vesicles with a different biomarker or biosignature. In some cases, hundreds or thousands of vesicles are assessed in a single assay.

In some embodiments, multiplex analysis is performed by applying a plurality of vesicles comprising more than one population of vesicles to a plurality of substrates, such as beads. Each bead is coupled to one or more capture agents. The plurality of beads is divided into subsets, where beads with the same capture agent or combination of capture agents form a subset of beads, such that each subset of beads has a different capture agent or combination of capture agents than another subset of beads. The beads can then be used to capture vesicles that comprise a component that binds to the capture agent. The different subsets can be used to capture different populations of vesicles. The captured vesicles can then be analyzed by detecting one or more biomarkers.

Flow cytometry can be used in combination with a particle-based or bead based assay. Multiparametric immunoassays or other high throughput detection assays using bead coatings with cognate ligands and reporter molecules with specific activities consistent with high sensitivity automation can be used. For example, beads in each subset can be differentially labeled from another subset. In a particle based assay system, a binding agent or capture agent for a vesicle, such as a capture antibody, can be immobilized on addressable beads or microspheres. Each binding agent for each individual binding assay (such as an immunoassay when the binding agent is an antibody) can be coupled to a distinct type of microsphere (i.e., microbead) and the binding assay reaction takes place on the surface of the microspheres. Microspheres can be distinguished by different labels, for example, a microsphere with a specific capture agent would have a different signaling label as compared to another microsphere with a different capture agent. For example, microspheres can be dyed with discrete fluorescence intensities such that the fluorescence intensity of a microsphere with a specific binding agent is different than that of another microsphere with a different binding agent. Biomarkers bound by different capture agents can be differentially detected using different labels.

A microsphere can be labeled or dyed with at least 2 different labels or dyes. In some embodiments, the microsphere is labeled with at least 3, 4, 5, 6, 7, 8, 9, or 10 different labels. Different microspheres in a plurality of microspheres can have more than one label or dye, wherein various subsets of the microspheres have various ratios and combinations of the labels or dyes permitting detection of different microspheres with different binding agents. For example, the various ratios and combinations of labels and dyes can permit different fluorescent intensities. Alternatively, the various ratios and combinations maybe used to generate different detection patters to identify the binding agent. The microspheres can be labeled or dyed externally or may have intrinsic fluorescence or signaling labels. Beads can be loaded separately with their appropriate binding agents and thus, different vesicle populations can be isolated based on the different binding agents on the differentially labeled microspheres to which the different binding agents are coupled.

In some embodiments, multiplex analysis can be performed using a planar substrate, wherein the substrate comprises a plurality of capture agents. The plurality of capture agents can capture one or more populations of vesicles, and one or more biomarkers of the captured vesicles detected. The planar substrate can be a microarray or other substrate as further described herein.

Binding Agents

A vesicle may be isolated or detected using a binding agent for a novel component of a vesicle, such as an antibody for a novel antigen specific to a vesicle of interest. Novel antigens that are specific to a vesicle of interest may be isolated or identified using different test compounds of known composition bound to a substrate, such as an array or a plurality of particles, which can allow a large amount of chemical/structural space to be adequately sampled using only a small fraction of the space. The novel antigen identified can also serve as a biomarker for the vesicle. For example, a novel antigen identified for a cell-of-origin specific vesicle can be a useful biomarker.

The term “agent” or “reagent” as used in respect to contacting a sample can mean any entity designed to bind, hybridize, associate with or otherwise detect or facilitate detection of a target molecule, including target polypeptides, peptides, nucleic acid molecules, leptins, lipids, or any other biological entity that can be detected as described herein or as known in the art. Examples of such agents/reagents are well known in the art, and include but are not limited to universal or specific nucleic acid primers, nucleic acid probes, antibodies, aptamers, peptoid, peptide nucleic acid, locked nucleic acid, lectin, dendrimer, chemical compound, or other entities described herein or known in the art.

A binding agent can be identified by screening either a homogeneous or heterogeneous vesicle population against test compounds. Since the composition of each test compound on the substrate surface is known, this constitutes a screen for affinity elements. For example, a test compound array comprises test compounds at specific locations on the substrate addressable locations, and can be used to identify one or more binding agents for a vesicle. The test compounds can all be unrelated or related based on minor variations of a core sequence or structure. The different test compounds may include variants of a given test compound (such as polypeptide isoforms), test compounds that are structurally or compositionally unrelated, or a combination thereof.

A test compound can be a peptoid, polysaccharide, organic compound, inorganic compound, polymer, lipids, nucleic acid, polypeptide, antibody, protein, polysaccharide, or other compound. The test compound can be natural or synthetic. The test compound can comprise or consist of linear or branched heteropolymeric compounds based on any of a number of linkages or combinations of linkages (e.g., amide, ester, ether, thiol, radical additions, metal coordination, etc.), dendritic structures, circular structures, cavity structures or other structures with multiple nearby sites of attachment that serve as scaffolds upon which specific additions are made. These test compound can be spotted on a substrate or synthesized in situ, using standard methods in the art. In addition, the test compound can be spotted or synthesized in situ in combinations in order to detect useful interactions, such as cooperative binding.

The test compound can be a polypeptide with known amino acid sequence, thus, detection of a test compound binding with a vesicle can lead to identification of a polypeptide of known amino sequence that can be used as a binding agent. For example, a homogenous population of vesicles can be applied to a spotted array on a slide containing between a few and 1,000,000 test polypeptides having a length of variable amino acids. The polypeptides can be attached to the surface through the C-terminus. The sequence of the polypeptides can be generated randomly from 19 amino acids, excluding cysteine. The binding reaction can include a non-specific competitor, such as excess bacterial proteins labeled with another dye such that the specificity ratio for each polypeptide binding target can be determined. The polypeptides with the highest specificity and binding can be selected. The identity of the polypeptide on each spot is known, and thus can be readily identified. Once the novel antigens specific to the homogeneous vesicle population, such as a cell-of-origin specific vesicle is identified, such cell-of-origin specific vesicles may subsequently be isolated using such antigens in methods described hereafter.

An array can also be used for identifying an antibody as a binding agent for a vesicle. Test antibodies can be attached to an array and screened against a heterogeneous population of vesicles to identify antibodies that can be used to isolate or identify a vesicle. A homogeneous population of vesicles such as cell-of-origin specific vesicles can also be screened with an antibody array. Other than identifying antibodies to isolate or detect a homogeneous population of vesicles, one or more protein biomarkers specific to the homogenous population can be identified. Commercially available platforms with test antibodies pre-selected or custom selection of test antibodies attached to the array can be used. For example, an antibody array from Full Moon Biosystems can be screened using prostate cancer cell derived vesicles identifying antibodies to Bcl-XL, ERCC1, Keratin 15, CD81/TAPA-1, CD9, Epithelial Specific Antigen (ESA), and Mast Cell Chymase as binding agents, and the proteins identified can be used as biomarkers for the vesicles. The biomarker can be present or absent, underexpressed or overexpressed, mutated, or modified in or on a vesicle and used in characterizing a condition.

An antibody or synthetic antibody to be used as a binding agent can also be identified through a peptide array. Another method is the use of synthetic antibody generation through antibody phage display. M13 bacteriophage libraries of antibodies (e.g. Fabs) are displayed on the surfaces of phage particles as fusions to a coat protein. Each phage particle displays a unique antibody and also encapsulates a vector that contains the encoding DNA. Highly diverse libraries can be constructed and represented as phage pools, which can be used in antibody selection for binding to immobilized antigens. Antigen-binding phages are retained by the immobilized antigen, and the nonbinding phages are removed by washing. The retained phage pool can be amplified by infection of an Escherichia coli host and the amplified pool can be used for additional rounds of selection to eventually obtain a population that is dominated by antigen-binding clones. At this stage, individual phase clones can be isolated and subjected to DNA sequencing to decode the sequences of the displayed antibodies. Through the use of phase display and other methods known in the art, high affinity designer antibodies for vesicles can be generated.

Bead-based assays can also be used to identify novel binding agents to isolate or detect a vesicle. A test antibody or peptide can be conjugated to a particle. For example, a bead can be conjugated to an antibody or peptide and used to detect and quantify the proteins expressed on the surface of a population of vesicles in order to discover and specifically select for novel antibodies that can target vesicles from specific tissue or tumor types. Any molecule of organic origin can be successfully conjugated to a polystyrene bead through use of a commercially available kit according to manufacturer's instructions. Each bead set can be colored a certain detectable wavelength and each can be linked to a known antibody or peptide which can be used to specifically measure which beads are linked to exosomal proteins matching the epitope of previously conjugated antibodies or peptides. The beads can be dyed with discrete fluorescence intensities such that each bead with a different intensity has a different binding agent as described above.

For example, a purified vesicle preparation can be diluted in assay buffer to an appropriate concentration according to empirically determined dynamic range of assay. A sufficient volume of coupled beads can be prepared and approximately 1 μl of the antibody-coupled beads can be aliqouted into a well and adjusted to a final volume of approximately 50 μl. Once the antibody-conjugated beads have been added to a vacuum compatible plate, the beads can be washed to ensure proper binding conditions. An appropriate volume of vesicle preparation can then be added to each well being tested and the mixture incubated, such as for 15-18 hours. A sufficient volume of detection antibodies using detection antibody diluent solution can be prepared and incubated with the mixture for 1 hour or more. The beads can then be washed before the addition of detection antibody (biotin expressing) mixture composed of streptavidin phycoereythin. The beads can then be washed and vacuum aspirated several times before analysis on a suspension array system using software provided with an instrument. The identity of antigens that can be used to selectively extract the vesicles can then be elucidated from the analysis.

Assays using imaging systems can be used to detect and quantify proteins expressed on the surface of a vesicle in order to discover and specifically select for and enrich vesicles from specific tissue, cell or tumor types. Antibodies, peptides or cells conjugated to multiple well multiplex carbon coated plates can be used. Simultaneous measurement of many analytes in a well can be achieved through the use of capture antibodies arrayed on the patterned carbon working surface. Analytes can then be detected with antibodies labeled with reagents in electrode wells with an enhanced electro-chemiluminescent plate. Any molecule of organic origin can be successfully conjugated to the carbon coated plate. Proteins expressed on the surface of vesicles can be identified from this assay and can be used as targets to specifically select for and enrich vesicles from specific tissue or tumor types.

The binding agent can also be an aptamer to a specific target. The term “specific” as used herein in regards to a binding agent can mean that an agent has a greater affinity for its target than other targets, typically with a much great affinity, but does not require that the binding agent is absolutely specific for its target.

Microfluidics

The methods for isolating or identifying vesicles can be used in combination with microfluidic devices. The methods of isolating or detecting a vesicle, such as described herien, can be performed using a microfluidic device. Microfluidic devices, which may also be referred to as “lab-on-a-chip” systems, biomedical micro-electro-mechanical systems (bioMEMs), or multicomponent integrated systems, can be used for isolating and analyzing a vesicle. Such systems miniaturize and compartmentalize processes that allow for binding of vesicles, detection of biosignatures, and other processes.

A microfluidic device can also be used for isolation of a vesicle through size differential or affinity selection. For example, a microfluidic device can use one more channels for isolating a vesicle from a biological sample based on size or by using one or more binding agents for isolating a vesicle from a biological sample. A biological sample can be introduced into one or more microfluidic channels, which selectively allows the passage of a vesicle. The selection can be based on a property of the vesicle, such as the size, shape, deformability, or biosignature of the vesicle.

In some embodiments, a heterogeneous population of vesicles can be introduced into a microfluidic device, and one or more different homogeneous populations of vesicles can be obtained. For example, different channels can have different size selections or binding agents to select for different vesicle populations. Thus, a microfluidic device can isolate a plurality of vesicles wherein at least a subset of the plurality of vesicles comprises a different biosignature from another subset of the plurality of vesicles. For example, the microfluidic device can isolate at least 2, 3, 4, 5, 6, 7, 8, 9, 10, 15, 20, 25, 30, 40, 50, 60, 70, 80, 90, or 100 different subsets of vesicles, wherein each subset of vesicles comprises a different biosignature.

In some embodiments, the microfluidic device can comprise one or more channels that permit further enrichment or selection of a vesicle. A population of vesicles that has been enriched after passage through a first channel can be introduced into a second channel, which allows the passage of the desired vesicle or vesicle population to be further enriched, such as through one or more binding agents present in the second channel.

Array-based assays and bead-based assays can be used with microfluidic device. For example, the binding agent can be coupled to beads and the binding reaction between the beads and vesicle can be performed in a microfluidic device. Multiplexing can also be performed using a microfluidic device. Different compartments can comprise different binding agents for different populations of vesicles, where each population is of a different cell-of-origin specific vesicle population. In some embodiments, each population has a different biosignature. The hybridization reaction between the microsphere and vesicle can be performed in a microfluidic device and the reaction mixture can be delivered to a detection device. The detection device, such as a dual or multiple laser detection system can be part of the microfluidic system and can use a laser to identify each bead or microsphere by its color-coding, and another laser can detect the hybridization signal associated with each bead.

Various microfluidic devices and methods are described above.

Combined Isolation Methodology

One of skill will appreciate that various methods of sample treatment and isolating and concentrating circulating biomarkers such as vesicles can be combined as desired. For example, a biological sample can be treated to prevent aggregation, remove undesired particulate and/or deplete highly abundant proteins. The steps used can be chosen to optimize downstream analysis steps. Next, biomarkers such as vesicles can be isolated, e.g., by chromotography, centrifugation, density gradient, filtration, precipitation, or affinity techniques. Any number of the later steps can be combined, e.g., a sample could be subjected to one or more of chromotography, centrifugation, density gradient, filtration and precipitation in order to isolate or concentrate all or most microvesicles. In a subsequent step, affinity techniques, e.g., using binding agents to one or more target of interest, can be used to isolate or identify microvesicles carrying desired biomarker profiles. Microfluidic systems can be employed to perform some or all of these steps.

An exemplary yet non-limiting isolation scheme for isolating and analysis of microvesicles includes the following: Plasma or serum collection->highly abundant protein removal->ultrafiltration->nanomembrane concentration->flow cytometry or particle-based assay.

Using the methods disclosed herein or known in the art, circulating biomarkers such as vesicles can be isolated or concentrated by at least about 2-fold, 3-fold, 1-fold, 2-fold, 3-fold, 4-fold, 5-fold, 6-fold, 7-fold, 8-fold, 9-fold, 10-fold, 12-fold, 15-fold, 20-fold, 25-fold, 30-fold, 35-fold, 40-fold, 45-fold, 50-fold, 55-fold, 60-fold, 65-fold, 70-fold, 75-fold, 80-fold, 90-fold, 95-fold, 100-fold, 110-fold, 120-fold, 125-fold, 130-fold, 140-fold, 150-fold, 160-fold, 170-fold, 175-fold, 180-fold, 190-fold, 200-fold, 205-fold, 250-fold, 275-fold, 300-fold, 325-fold, 350-fold, 375-fold, 400-fold, 425-fold, 450-fold, 475-fold, 500-fold, 525-fold, 550-fold, 575-fold, 600-fold, 625-fold, 650-fold, 675-fold, 700-fold, 725-fold, 750-fold, 775-fold, 800-fold, 825-fold, 850-fold, 875-fold, 900-fold, 925-fold, 950-fold, 975-fold, 1000-fold, 1500-fold, 2000-fold, 2500-fold, 3000-fold, 4000-fold, 5000-fold, 6000-fold, 7000-fold, 8000-fold, 9000-fold, or at least 10,000-fold. In some embodiments, the vesicles are isolated or concentrated concentrated by at least 1 order of magnitude, 2 orders of magnitude, 3 orders of magnitude, 4 orders of magnitude, 5 orders of magnitude, 6 orders of magnitude, 7 orders of magnitude, 8 orders of magnitude, 9 orders of magnitude, or 10 orders of magnitude or more.

Once concentrated or isolated, the circulating biomarkers can be assessed, e.g., in order to characterize a phenotype as described herein. In some embodiments, the concentration or isolation steps themselves shed light on the phenotype of interest. For example, affinity methods can detect the presence or level of specific biomarkers of interest.

The various isolation and detection systems described herein can be used to isolate or detect circulating biomarkers such as vesicles that are informative for diagnosis, prognosis, disease stratification, theranosis, prediction of responder/non-responder status, disease monitoring, treatment monitoring and the like as related to such diseases and disorders. Combinations of the isolation techniques are within the scope of the invention. In a non-limiting example, a sample can be run through a chromatography column to isolate vesicles based on a property such as size of electrophoretic motility, and the vesicles can then be passed through a microfluidic device. Binding agents can be used before, during or after these steps.

The methods and compositions of the invention can be used with microvesicles isolated or detected using such methods as described herein. In various non-limiting examples: an aptamer is used as a capture and/or detector agent for a biomarker such as a protein or microvesicle; a sample such as a bodily fluid can be contacted with an oligonucleotide probe library before microvesicles in the sample are isolated using the methods provided herein. See, e.g., Example 3. Alternately, the microvesicles can be isolated prior to contact with the aptamer library. Additional isolation steps may be performed as desired, e.g., one or more technique described herein (e.g., chromatography, centrifugation, flow cytometry, filtration, affinity isolation, polymer precipitation, etc). Contaminants such as highly abundant proteins can be removed in whole or in part at any appropriate step in such processes. These and various other useful iterations of such techniques for assessment of microvesicles and other biomarkers are contemplated by the invention.

### Biomarkers

As described herein, the methods and compositions of the invention can be used in assays to detect the presence or level of one or more biomarker of interest. The biomarker can be any useful biomarker disclosed herein or known to those of skill in the art. In some embodiments, the biomarker comprises a protein or polypeptide. As used herein, “protein,” “polypeptide” and “peptide” are used interchangeably unless stated otherwise. The biomarker can be a nucleic acid, including DNA, RNA, and various subspecies of any thereof as disclosed herein or known in the art. The biomarker can comprise a lipid. The biomarker can comprise a carbohydrate. The biomarker can also be a complex, e.g., a complex comprising protein, nucleic acids, lipids and/or carbohydrates. In some embodiments, the biomarker comprises a microvesicle. In some embodiments, the invention provides a method wherein a pool of aptamers is used to assess the presence and/or level of a population of microvesicles of interest without knowing the precise microvesicle antigen targeted by each member of the pool. See, e.g., FIGS. 2E-F. In other cases, biomarkers associated with microvesicles isolated using the methods provided herein are assessed. See, e.g., FIGS. 1A-E; FIG. 2D.

A biosignature comprising more than one biomarker can comprise one type of biomarker or multiple types of biomarkers. As a non-limiting example, a biosignature can comprise multiple proteins, multiple nucleic acids, multiple lipids, multiple carbohydrates, multiple biomarker complexes, multiple microvesicles, or a combination of any thereof. For example, the biosignature may comprise one or more microvesicle, one or more protein, and one or more microRNA, wherein the one or more protein and/or one or more microRNA is optionally in association with the microvesicle as a surface antigen and/or payload, as appropriate.

In some embodiments, vesicles are detected using vesicle surface antigens. A commonly expressed vesicle surface antigen can be referred to as a “housekeeping protein,” or general vesicle biomarker. The biomarker can be CD63, CD9, CD81, CD82, CD37, CD53, Rab-5b, Annexin V or MFG-E8. Tetraspanins, a family of membrane proteins with four transmembrane domains, can be used as general vesicle biomarkers. The tetraspanins include CD151, CD53, CD37, CD82, CD81, CD9 and CD63. There have been over 30 tetraspanins identified in mammals, including the TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231, TALLA-1, A15), TSPAN8 (CO-029), TSPAN9 (NET-5), TSPAN10 (Oculospanin), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UP1b, UPK1B), TSPAN21 (UPla, UPK1A), TSPAN22 (RDS, PRPH2), TSPAN23 (ROM1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31 (SAS), TSPAN32 (TSSC6), TSPAN33, and TSPAN34. Other commonly observed vesicle markers include those listed in Table 3. One or more of these proteins can be useful biomarkers for the characterizing a phenotype using the subject methods and compositions.

TABLE 3
Proteins Observed in Vesicles from Multiple Cell Types

| Class | Protein |
|---|---|
| Antigen Presentation | MHC class I, MHC class II, Integrins, Alpha 4 beta 1, Alpha M beta 2, |
| | Beta 2 |
| Immunoglobulin family | ICAM1/CD54, P-selection |
| Cell-surface peptidases | Dipeptidylpeptidase IV/CD26, Aminopeptidase n/CD13 |
| Tetraspanins | CD151, CD53, CD37, CD82, CD81, CD9 and CD63 |
| Heat-shock proteins | Hsp70, Hsp84/90 |
| Cytoskeletal proteins | Actin, Actin-binding proteins, Tubulin |
| Membrane transport | Annexin I, Annexin II, Annexin IV, Annexin V, Annexin VI, |
| and fusion | RAB7/RAP1B/RADGDI |
| Signal transduction | Gi2alpha/14-3-3, CBL/LCK |
| Abundant membrane | CD63, GAPDH, CD9, CD81, ANXA2, ENO1, SDCBP, MSN, MFGE8, |
| proteins | EZR, GK, ANXA1, LAMP2, DPP4, TSG101, HSPA1A, GDI2, CLTC, |
| | LAMP1, Cd86, ANPEP, TFRC, SLC3A2, RDX, RAP1B, RAB5C, |
| | RAB5B, MYH9, ICAM1, FN1, RAB11B, PIGR, LGALS3, ITGB1, |
| | EHD1, CLIC1, ATP1A1, ARF1, RAP1A, P4HB, MUC1, KRT10, HLA- |
| | A, FLOT1, CD59, C1orf58, BASP1, TACSTD1, STOM |
| Other Transmembrane | Cadherins: CDH1, CDH2, CDH12, CDH3, Deomoglein, DSG1, DSG2, |
| Proteins | DSG3, DSG4, Desmocollin, DSC1, DSC2, DSC3, Protocadherins, |
| | PCDH1, PCDH10, PCDH11x, PCDH11y, PCDH12, FAT, FAT2, FAT4, |
| | PCDH15, PCDH17, PCDH18, PCDH19; PCDH20; PCDH7, PCDH8, |
| | PCDH9, PCDHA1, PCDHA10, PCDHA11, PCDHA12, PCDHA13, |
| | PCDHA2, PCDHA3, PCDHA4, PCDHA5, PCDHA6, PCDHA7, |
| | PCDHA8, PCDHA9, PCDHAC1, PCDHAC2, PCDHB1, PCDHB10, |
| | PCDHB11, PCDHB12, PCDHB13, PCDHB14, PCDHB15, PCDHB16, |
| | PCDHB17, PCDHB18, PCDHB2, PCDHB3, PCDHB4, PCDHB5, |
| | PCDHB6, PCDHB7, PCDHB8, PCDHB9, PCDHGA1, PCDHGA10, |
| | PCDHGA11, PCDHGA12, PCDHGA2; PCDHGA3, PCDHGA4, |
| | PCDHGA5, PCDHGA6, PCDHGA7, PCDHGA8, PCDHGA9, |
| | PCDHGB1, PCDHGB2, PCDHGB3, PCDHGB4, PCDHGB5, |
| | PCDHGB6, PCDHGB7, PCDHGC3, PCDHGC4, PCDHGC5, CDH9 |
| | (cadherin 9, type 2 (T1-cadherin)), CDH10 (cadherin 10, type 2 (T2- |
| | cadherin)), CDH5 (VE-cadherin (vascular endothelial)), CDH6 (K- |
| | cadherin (kidney)), CDH7 (cadherin 7, type 2), CDH8 (cadherin 8, type |
| | 2), CDH11 (OB-cadherin (osteoblast)), CDH13 (T-cadherin - H-cadherin |
| | (heart)), CDH15 (M-cadherin (myotubule)), CDH16 (KSP-cadherin), |
| | CDH17 (LI cadherin (liver-intestine)), CDH18 (cadherin 18, type 2), |
| | CDH19 (cadherin 19, type 2), CDH20 (cadherin 20, type 2), CDH23 |
| | (cadherin 23, (neurosensory epithelium)), CDH10, CDH11, CDH13, |
| | CDH15, CDH16, CDH17, CDH18, CDH19, CDH22, CDH23, CDH24, |
| | CDH26, CDH28, CDH4, CDH5, CDH6, CDH7, CDH8, CDH9, |
| | CELSR1, CELSR2, CELSR3, CLSTN1, CLSTN2, CLSTN3, DCHS1, |
| | DCHS2, LOC389118, PCLKC, RESDA1, RET |

Any of the types of biomarkers or specific biomarkers described herein can be used and/or assessed via the subject methods and compositions, e.g., to identify a useful biosignature. Exemplary biomarkers include without limitation those in Table 6. The markers can be detected as protein, RNA or DNA as appropriate, which can be circulating freely or in a complex with other biological molecules. As appropriate, the chosen can also be used for capture and/or detection of vesicles for characterizing phenotypes as disclosed herein. In some cases, multiple capture and/or detectors are used to enhance the characterization. The markers can be detected as protein or as mRNA, which can be circulating freely or in a complex with other biological molecules. See, e.g., FIGS. 1C-E. The markers can be detected as vesicle surface antigens and/or vesicle payload. The “Illustrative Class” indicates indications for which the markers are known markers. Those of skill will appreciate that the markers can also be used in alternate settings in certain instances. For example, a marker which can be used to characterize one type disease may also be used to characterize another disease as appropriate. Consider a non-limiting example of a tumor marker which can be used as a biomarker for tumors from various lineages. The biomarker references in Tables 3 and Table 6 are those commonly used in the art. Gene aliases and descriptions can be found using a variety of online databases, including GeneCards® (www.genecards.org), HUGO Gene Nomenclature (www.genenames.org), Entrez Gene (www.ncbi.nlm.nih.gov/entrez/query.fcgi?db=gene), UniProtKB/Swiss-Prot (www.uniprot.org), UniProtKB/TrEMBL (www.uniprot.org), OMIM (www.ncbi.nlm.nih.gov/entrez/query.fcgi?db=OMIM), GeneLoc (genecards.weizmann.ac.il/geneloc/), and Ensembl (www.ensembl.org). Generally, gene symbols and names below correspond to those approved by HUGO, and protein names are those recommended by UniProtKB/Swiss-Prot.

Examples of additional biomarkers that can be incorporated into the methods and compositions of the invention include without limitation those disclosed in International Patent Application Nos. PCT/US09/06095, entitled “Methods and Systems of Using Exosomes for Determining Phenotypes” and filed Nov. 12, 2009; PCT/US2011/031479, entitled “Circulating Biomarkers for Disease” and filed Apr. 6, 2011; PCT/US2012/025741, filed Feb. 17, 2012; PCT/US2011/048327, filed Aug. 18, 2011; PCT/US2011/026750, filed Mar. 1, 2011; PCT/US2012/042519 (WO 2012/174282), filed Jun. 14, 2012; PCT/US2012/050030 (WO 2013/022995), filed Aug. 8, 2012; PCT/US2010/058461, entitled “Methods and Systems for Isolating, Storing, and Analyzing Vesicles” and filed Nov. 30, 2010; and PCT/US2011/021160, entitled “Detection of Gastrointestinal Disorders” and filed Jan. 13, 2011; each of which applications is incorporated by reference herein in its entirety.

In various embodiments of the invention, the biomarkers or biosignature used to detect or assess any of the conditions or diseases disclosed herein can comprise one or more biomarkers in one of several different categories of markers, wherein the categories include without limitation one or more of: 1) disease specific biomarkers; 2) cell- or tissue-specific biomarkers; 3) vesicle-specific markers (e.g., general vesicle biomarkers); 4 angiogenesis-specific biomarkers; and 5) immunomodulatory biomarkers. Examples of all such markers are disclosed herein and known to a person having ordinary skill in the art. Furthermore, a biomarker known in the art that is characterized to have a role in a particular disease or condition can be adapted for use as a target in compositions and methods of the invention. In further embodiments, such biomarkers that are associated with vesicles can be all vesicle surface markers, or a combination of vesicle surface markers and vesicle payload markers (i.e., molecules enclosed by a vesicle). The biomarkers assessed can be from a combination of sources. For example, a disease or disorder may be detected or characterized by assessing a combination of proteins, nucleic acids, vesicles, circulating biomarkers, biomarkers from a tissue sample, and the like. In addition, as noted herein, the biological sample assessed can be any biological fluid, or can comprise individual components present within such biological fluid (e.g., vesicles, nucleic acids, proteins, or complexes thereof).

EpCAM is a pan-epithelial differentiation antigen that is expressed on many tumor cells. It is intricately linked with the Cadherin-Catenin pathway and hence the fundamental WNT pathway responsible for intracellular signalling and polarity. It has been used as an immunotherapeutic target in the treatment of gastrointestinal, urological and other carcinomas. (Chaudry M A, Sales K, Ruf P, Lindhofer H, Winslet M C (April 2007). Br. J. Cancer 96 (7): 1013-9.). It is expressed in undifferentiated pluripotent stem cells. EpCAM is a member of a family that includes at least two type I membrane proteins and functions as a homotypic calcium-independent cell adhesion molecule. Mutations in this gene result in congenital tufting enteropathy. EpCAM has been observed on the surface of microvesicles derived from cancer cell of various lineages. EpCAM is used as an exemplary surface antigen in various examples herein. One of skill will appreciate that various embodiments and examples using EpCAM can be applied to other microvesicle surface antigens as well.

### Aptamer Library Detection Methods

Aptamers are oligomeric nucleic acid molecules having specific binding affinity to molecules, which may be through interactions other than classic Watson-Crick base pairing. Unless otherwise specified, an “aptamer” as the term is used herein can refer to nucleic acid molecules that can be used to characterize a phenotype, regardless of manner of target recognition. Unless other specified, the terms “aptamer,” “oligonucleotide,” “polynucleotide,” or the like may be used interchangeably herein.

Aptamers, like peptides generated by phage display or monoclonal antibodies (“mAbs”), are capable of specifically binding to selected targets and modulating the target's activity, e.g., through binding aptamers may block their target's ability to function. Created by an in vitro selection process from pools of random sequence oligonucleotides, aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., aptamers will typically not bind other proteins from the same gene family). A series of structural studies have shown that aptamers are capable of using the same types of binding interactions (e.g., hydrogen bonding, electrostatic complementarity, hydrophobic contacts, steric exclusion) that drive affinity and specificity in antibody-antigen complexes.

Extracellular vesicles provide an attractive vehicle to profile the biological complexity and diversity driven by many inter-related sources. There can be a great deal of heterogeneity between patient-to-patient microvesicle populations, or even in microvesicle populations from a single patient under different conditions (e.g., stress, diet, exercise, rest, disease, etc). Diversity of molecular phenotypes within microvesicle populations in various disease states, even after microvesicle isolation and sorting by vesicle biomarkers, can present challenges identifying surface binding ligands. This situation is further complicated by vesicle surface-membrane protein complexes. The oligonucleotide probe library can be used to address such challenges and allow for characterization of biological phenotypes. The approach combines the power of diverse oligonucleotide libraries and high throughput (next-generation) sequencing technologies to probe the complexity of extracellular microvesicles.

Aptamer library profiling may provide quantitative measurements of dynamic events in addition to detection of presence/absence of various biomarkers in a sample. For example, the binding probes may detect protein complexes or other post-translation modifications, allowing for differentiation of samples with the same proteins but in different biological configurations. Such configuration is illustrated in FIG. 2A. In FIG. 2A, microvesicles with various surface markers are shown from an example microvesicle sample population: Sample Population A. The indicated Bound Probing Oligonucleotide aptamers 2001 are contacted to two surface markers 2002 and 2003 in a given special relationship. Here, probes unique to these functional complexes and spatial relationships may be retained. In contrast, in another microvesicle population, the two surface markers 2002 and 2003 may be found in disparate spacial relationship. In such case, probes 2001 are not bound due to absence of the spatial relationship of the interacting components 2002 and 2003.

An illustrative approach 2010 for using aptamer library profiling to assess a sample is shown in FIG. 2B. The probing library 2011 is mixed with sample 2012. The sample can be as described herein, e.g., a bodily fluid from a subject having or suspected of having a disease. The probes are allowed to bind the sample 2020 and the microvesicles are pelleted 2015. The microvesicles may be isolated using the methods provided herein before or after contact with the probes. The supernatant 2014 comprising unbound oligonucleotides is discarded. Oligonucleotide probes bound to the pellet 2015 are eluted 2016 and sequenced 2017. The profile 2018 generated by the bound oligonucleotide probes as determined by the sequencing 2017 is used to characterize the sample 2012. For example, the profile 2018 can be compared to a reference, e.g., to determine if the profile is similar or different from a reference profile indicative of a disease or healthy state, or other phenotypic characterization of interest. The comparison may indicate the presence of a disease, provide a diagnosis, prognosis or theranosis, or otherwise characterize a phenotype associated with the sample 2012. FIG. 2C illustrates another schematic for using aptamer library profiling to characterize a phenotype. A patient sample such as a bodily fluid disclosed herein is collected 2021. The sample is contacted with the aptamer library pool 2022. Microvesicles (MVs) are isolated from the contacted sample 2023, e.g., using ultracentrifugation, filtration, polymer precipitation or other appropriate technique or combination of techniques disclosed herein. Oligonucleotides that bound the isolated microvesicles are collected and identity is determined 2024. The identity of the bound oligonucleotides can be determined by any useful technique such as sequencing, high throughput sequencing (e.g., NGS), amplification including without limitation qPCR, or hybridization such as to a planar or particle based array. The identity of the bound oligonucleotides is used to characterize the sample, e.g., as containing disease related microvesicles.

FIG. 2D is a schematic 2030 showing an assay configuration that can be used to detect and/or quantify a target of interest using one or more aptamer of the invention. Capture aptamer 2032 is attached to substrate 2031. The substrate can be a planar substrate, well, microbead, or other useful substrate as disclosed herein or known in the art. Target of interest 2033, e.g., a microvesicle, is bound by capture aptamer 2032. The target of interest can be any appropriate entity that can be detected when recognized by an aptamer or other binding agent. The target of interest may comprise a protein or polypeptide, a nucleic acid, including DNA, RNA, and various subspecies thereof, a lipid, a carbohydrate, a complex, e.g., a complex comprising protein, nucleic acids, lipids and/or carbohydrates. In some embodiments, the target of interest comprises a microvesicle. The target of interest can be a microvesicle surface antigen. The target of interest may be a biomarker, including a vesicle associated biomarker, in Table 3 or Table 6. The microvesicle input can be isolated from a sample using the methods provided herein, solely or in combination with other sample processing techniques such chromatography, filtration, centrifugation, flow cytometry, affinity capture (e.g., to a planar surface, column or bead), and/or using microfluidics. Detection aptamer 2034 is also bound to target of interest 2033. Detection aptamer 2034 carries label 2035 which can be detected to identify target captured to substrate 2031 via capture aptamer 2032. The label can be a fluorescent, radiolabel, enzyme, or other detectable label as disclosed herein. Either capture aptamer 2032 or detection aptamer 2034 can be substituted with another binding agent, e.g., an antibody. For example, the target may be captured with an antibody and detected with an aptamer, or vice versa. When the target of interest comprises a complex, the capture and detection agents (aptamer, antibody, etc) can recognize the same or different targets. For example, when the target is a microvesicle, the capture agent may recognize one microvesicle surface antigen while the detection agent recognizes another microvesicle surface antigen. Alternately, the capture and detection agents can recognize the same surface antigen.

FIG. 2E is a schematic 2040 showing use of an aptamer library to characterize a phenotype of a sample, such as microvesicles isolated as provided herein. A pool of oligonucleotide aptamers to a target of interest is provided 2041. For example, the pool of oligonucleotides can be enriched to target one or more microvesicle. The members of the pool may bind different targets (e.g., a microvesicle surface antigen) or different epitopes of the same target present on the one or more microvesicle. The pool is contacted with a test sample to be characterized 2042. For example, the test sample may be a biological sample from an individual having or suspected of having a given disease or disorder. The mixture is washed to remove unbound oligonucleotides. The remaining oligonucleotides are eluted or otherwise disassociated from the sample and collected 2043. The collected oligonucleotides are identified, e.g., by sequencing or hybridization 2044. The presence and/or copy number of the identified is used to characterize the phenotype 2045. For example, the pool of oligonucleotides may be chosen as oligonucleotides that preferentially recognize microvesicles shed from cancer cells. The method can be employed to detect whether the sample retains oligonucleotides that bind the cancer-related microvesicles, thereby allowing the sample to be characterized as cancerous or not.

FIG. 2F is a schematic 2050 showing an implementation of the method in FIG. 2E. A pool of oligonucleotides identified as binding a microvesicle population is provided 2051. The input sample comprises a test sample comprising microvesicles 2052. For example, the test sample may be a biological sample from an individual having or suspected of having a given disease or disorder. The pool is contacted with the isolated microvesicles to be characterized 2053. The microvesicle population can be isolated before or after the contacting 2053 from the sample using the methods provided herein, alone or in addition to alternate isolation techniques such as, e.g., chromatography, filtration, ultrafiltration, centrifugation, ultracentrifugation, flow cytometry, affinity capture (e.g., to a planar surface, column or bead), PEG precipitation, and/or using microfluidics. The mixture is washed to remove unbound oligonucleotides and the remaining oligonucleotides are eluted or otherwise disassociated from the sample and collected 2054. The collected oligonucleotides are identified 2055 and the presence and/or copy number of the retained oligonucleotides is used to characterize the phenotype 2056 as above.

As noted, in embodiment of FIG. 2F, the pool of oligonucleotides 2051 is directly contacted with a biological sample that comprises or is expected to comprise microvesicles 2052. Microvesicles are thereafter isolated from the sample and the mixture is washed to remove unbound oligonucleotides and the remaining oligonucleotides are disassociated and collected 2054. The following steps are performed as above. As an example of this alternate configuration, a biological sample, e.g., a blood, serum or plasma sample, is directly contacted with the pool of oligonucleotides. Microvesicles are then isolated by various techniques as desired, including without limitation the methods provided herein. The methods of invention may be used in combination with other techniques if desired, e.g., ultracentrifugation, ultrafiltration, flow cytometry, affinity isolation, polymer precipitation, chromatography, various combinations thereof, or the like. Remaining oligonucleotides are then identified, e.g., by sequencing, hybridization or amplification.

The methods of isolating microvesicles provided herein can be used to provide such microvesicles for use as targets of such aptamer libraries/oligonucleotide pools. See Domenyuk, V. et al. Plasma Exosome Profiling of Cancer Patients by a Next Generation Systems Biology Approach. Sci Rep 7, 42741, doi:10.1038/srep42741 (2017); Domenyuk, V. et al. Poly-ligand profiling differentiates trastuzumab-treated breast cancer patients according to their outcomes. Nat. Commun. 9, 1219, doi:10.1038/s41467-018-03631-z (2018); Int'l Patent Publications WO/2014/068408, published May 8, 2014 (based on Int'l Patent Appl. PCT/IB2013/003092, filed Oct. 23, 2013); WO/2015/031694, published Mar. 5, 2015 (based on Int'l Patent Appl. PCT/US14/53306, filed Aug. 28, 2014); WO/2016/081941, published May 26, 2016 (based on Int'l Patent Appl. PCT/US15/62184, filed Nov. 23, 2015); WO/2016/145128, published Sep. 15, 2016 (based on Int'l Patent Appl. PCT/US16/21632, filed Mar. 9, 2016); WO/2017/161357, published Sep. 21, 2017 (based on Int'l Patent Appl. PCT/US17/23108, filed Mar. 18, 2017); and WO/2017/205686, published Nov. 30, 2017 (based on Int'l Patent Appl. PCT/US17/34567, filed May 25, 2017); all of which references are incorporated by reference herein in their entirety.

### Kits

The invention also provides a kit comprising one or more reagent to carry out the methods provided herein. For example, the one or more reagent can be the one or more binding agent, a buffer, blocker, enzyme, or combination thereof. The one or more reagent may comprise any useful reagents for carrying out the subject methods, including without limitation pluronic block co-polymer, e.g., F68 or F127. If desired, the one or more reagent can be a device to facilite additional isolation steps (e.g., via chromatography, filtration, ultrafiltration, centrifugation, ultracentrifugation, flow cytometry, affinity capture (e.g., to a planar surface, column or bead), polymer precipitation, and/or using microfluidics). In some embodiments, the kit contains reagents for analysis or characterization of the microvesicles. For example, aptamers directed to specific targets, aptamer pools that facilitate detection of a biomarker/microvesicle population, reagents such as primers for nucleic acid sequencing or amplification, arrays for nucleic acid hybridization, detectable labels, solvents or buffers and the like, various linkers, various assay components, blockers, and the like. The one or more reagent can comprise a substrate, such as a planar substrate, column or bead. The kit can contain instructions to carry out various assays using the one or more reagent.

In some embodiments, the kit comprises an oligonucleotide probe or composition provided herein. For example, the kit can include an aptamer library, a substrate, or both an aptamer library and a substrate. The kit may include additional reagents useful for assessing members of the aptamer library that bind or not to vesicles isolated using the methods provided herein.

In some embodiments, the kit is configured to carry out an assay. For example, the kit can contain one or more reagent and instructions for detecting the presence or level of a biological entity, e.g., a microvesicle population, in a biological sample. In such cases, the kit can include one or more binding agent to a biological entity of interest. The one or more binding agent can be bound to a substrate.

In some embodiments, the kit comprises a set of oligonucleotides that provide a particular oligonucleotide profile for a biological sample. An oligonucleotide profile can include, without limitation, a profile that can be used to characterize a particular disease or disorder. For example, the disease or disorder can be a proliferative disease or disorder, including without limitation a cancer. In some embodiments, the cancer comprises a breast cancer.

### Examples

### Example 1: Aptamer Based Precipitation of Microvesicles in Plasma

Aptamers can specifically recognize target proteins with nanomolar affinity and have some potential advantages compared to antibodies due to their chemical stability, ease of synthesis and overall reproducibility. Microvesicles were isolated from a rat glioma cell line model that expresses or does not express the human EGFR protein in order to optimize aptamer-microvesicle complex formation and isolation in plasma. Microvesicles spiked into human plasma were precipitated with the positive EGFR DNA or RNA aptamer but not with a reverse complement control sequence. There was no observed binding of the positive or negative aptamer to microvesicles that do not express EGFR. Binding of aptamers to microvesicles was confirmed with aptamer based ELISAs, EMSA assays, and flow cytometry. These results demonstrate aptamer-based precipitation of microvesicles from a complex biological sample.

### Example 2: Quantitative Proteomics of EGFR, EGFRviii, and EGFR Negative Microvesicles from a Rat Glioma Cell Line

In this Example, we performed quantitative proteomics on microvesicles isolated from a rat glioma cell line F98 (parental line), F98(EGFR) and F98(EGFRviii). Different lots of microvesicles purified from each cell line type were considered biological replicates and each biological replicate was analyzed in four technical replicates by tandem mass tag 6-plex (TMT) peptide labeling and LC-MS/MS. The general microvesicle markers CD81, CD9, CD63, Tsg101, and Alix were identified in all technical replicates. Multiple proteins were found to be up or down-regulated including a 7.5-fold increase in EGFR and a 2.6-fold increase in the EGFR binding partner HMCN2 in the F98 [EGFR] compared with the parental line. These results indicate that expression of a single biomarker may affect global proteomic changes in microvesicles.

### Example 3: Profiling Extracellular Vesicle by Pluronic Block-Copolymer Based Enrichment

Extracellular vesicle (EV; also referred to as microvesicles, exosomes, etc.) are found in bodily fluids and provide a source material to examine biomarker profiles, e.g., for use in liquid biopsies or other diagnostics purposes. Development of a fast and reliable preparation protocol to enrich such small particles could accelerate the discovery of informative, disease-related biomarkers. Though multiple EV enrichment protocols are available, in terms of efficiency, reproducibility and simplicity, precipitation based methods are advantageous for laboratory testing or for studies with large numbers of subjects. For example, such methods may be more rapid and less equipment intensive than classical ultracentrifugation (UC) based methods. However, the selectivity of the precipitation process can vary. In this Example, we present a plasma EV enrichment protocol based on pluronic block copolymer. The enriched plasma EV was verified by multiple platforms. Our results showed that the particles enriched from plasma by the copolymer were EV size vesicles with membrane structure. Proteomic profiling of the enriched vesicles showed that EV related proteins were significantly enriched, while high abundant plasma proteins were significantly reduced in comparison to other precipitation based enrichment methods. Next generation sequencing (NGS) confirmed the existence of various RNA species that have been observed in EVs from previous studies. Small RNA sequencing showed enriched species compared to the corresponding plasma. Moreover, plasma EVs enriched from 20 advanced breast cancer patients and 20 age-matched non-cancer controls were profiled by semi-quantitative mass spectrometry. Protein features were further screened by EV proteomic profiles generated from four breast cancer cell lines, and then selected in cross-validation models. Sixty protein features that highly contributed in model prediction were identified. A portion of these features were associated with breast cancer aggression, metastasis and/or invasion, which findings are consistent to the advanced clinical stage of the patients. In summary, we have developed a plasma EV enrichment method with improved precipitation selectivity and suitable for larger scale studies.

Introduction

Extracellular vesicles (EVs), or exosomes, are small, spherical vesicles with a diameter of ˜40-100 nm that are secreted into the extracellular milieu by many cell types upon fusing the membrane of multi-vesicular bodies (MVBs) with the plasma membrane. The larger class of micro-particles (MPs), including EVs, micro-vesicles (MVs), apoptotic bodies and apoptotic micro-particles, are typically small particles ranging from 50 nm to 3 μm in diameter. MPs released from the surface of plasma membranes may be referred to as microvesicles, membrane particles, as well as apoptotic vesicles [1]. EV release may be a highly regulated process instead of a random process for the removing unwanted internal cellular debris [2]. EVs located in biological fluids attract enormous interest because of their potential use as a source of protein and nucleic acid biomarkers, or as a prospective delivery system for various therapeutic purposes [3]. An efficient method for the enrichment of EV from complex biofluids, such as plasma samples, that addresses the many pitfalls of currently used methods would be useful in the research, clinical, and commercial setting.

MPs are found in various bodily fluids. Such bodily fluids, including without limitation blood samples, frequently contain large quantities of soluble proteins, aggregates and contaminants from other organelles that restrict the accuracy of EV analysis. Limitations of current methods in enriching EVs from complex biological fluids include sample volume requirements, carryover of undesired high abundance proteins, and interference of downstream analysis due to leftover of method-based reagents. Differential ultracentrifugation (UC) has been the gold standard to separate EVs [4], and facilitates the removal of most of the plasma contaminants. However, time consuming steps and large sample volume requirement leads to poor reproducibility and restrict its utilities for large scale studies. Immuno-precipitation (IP) of MPs is another option. Although providing faster processing than UC, IP is restricted to certain EV subpopulation with known surface proteins, which inter alia, limits its value to probe entire populations of MPs and/or identify novel biomarkers. More recently, polymer based enrichment techniques by, for example PEG8000, has been used to enrich EVs [5].

Previous studies have compared the reproducibility and efficiency in enriching plasma-derived EVs between UC and PEG-based methods [6, 7, 8]. Though results might vary from study to study, the conclusion in general was that UC may obtain EV with relatively better purity but with very low efficiency. In contrast, PEG-based method offers higher efficiency and better reproducibility, but in the cost of contamination of high abundant plasma proteins. High viscosity and carryover for PEG based method also might affect the compatibility to the downstream applications, such as electro-microscopy (EM), mass spectrometry (MS) and flow cytometry (FL). Procedures that used for the removal of the polymer are often tedious and incomplete.

Pluronic block copolymers (also referred to as poloxamers) consist of ethylene oxide (EO) and propylene oxide (PO) blocks arranged in a triblock structure: EOx-POy-EOx. This arrangement results in an amphiphilic copolymer, in which the hydrophilicity and hydrophobicity could be altered by varying the size of the hydrophilic EO(x) and hydrophobic PO(y) units [9]. Pluronic block copolymers F68 and related F127 have been reported to have broad utilities and biological properties. Copolymers with higher hydrophilic/lipophilic balance (HLB) (e.g., F-68, HLB0.80) were shown to be capable of being inserted into lipid bilayer membranes and able to restore the integrity of damaged membranes [10, 11, 12, 13]; F68 was also shown to be capable of inhibiting protein aggregation [14]. These biological properties ameliorate various issues with PEG precipitation, and offer the potential to increase the selectivity of EV precipitation when enriching from complex biofluids, such as plasma.

An ideal method of enriching EVs from might be a balance between high efficiency, low contamination and simplicity. We describe here an EV enrichment method based on the Pluronic block copolymer F68, which balances these three requirements. We used this process to enrich MPs from blood plasma samples. Compared to alternate methods, this enrichment method described is more selective for plasma EV fraction with high efficiency, while much less contamination from high abundant plasma protein. As an example of utility, plasma EVs from 20 high-grade (stage III and IV) breast cancer patients and age-matched non-cancer controls were enriched using the methods provided herein and profiled to identify features in the EVs that might be related to breast cancer aggression, metastasis and invasion.

### EXPERIMENTAL PROCEDURES

Cell Line EV Preparation

Cell lines used in this study: Vcap, MCF7, T47D, MDA-MB-231, MDA-MB-468. Cell culture: FBS (20% in DMEM) was depleted of bovine EV by centrifugation at 100,000 g for 16 h at 4° C. Vcap cells were cultured in DMEM, MCF7 cells were culture in EMEM, T47D cells were cultured in RMPI1640, MDA-MB-231/468 were cultured in L15 medium, all supplemented with 10% depleted FBS, 2 mM L-glutamine, 1 U/mL penicillin, and 1 μg/mL streptomycin at 37° C. and 5% CO2. Cell line generated EVs were isolated from cell cultures supernatant by sucrose density centrifugation. Briefly, supernatant was cleared of cells and cellular debris by sequential centrifugation at 400 g for 10 min and 2000 g for 20 min at 4° C. Cleared supernatant was concentrated by centrifugal filtration (Centricon Plus-70, 100 kDa NMWL), layered on a 30% sucrose cushion and centrifuged at 100,000 g for 75 min at 4° C. Supernatant was removed and discarded without disrupting the cushion interface, which was then collected, diluted 6-fold with PBS and centrifuged at 100,000 g for 70 min at 4° C. The resulting EV pellet was re-suspended in PBS by pipetting and incubation overnight at 4° C., and then stored at −80° C.

Breast Cancer/Non-Cancer Plasma Samples and Processing

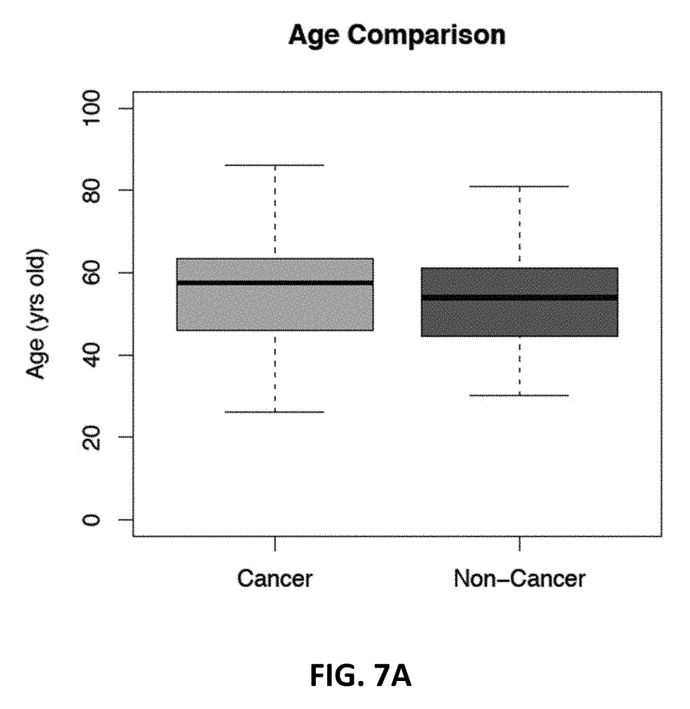

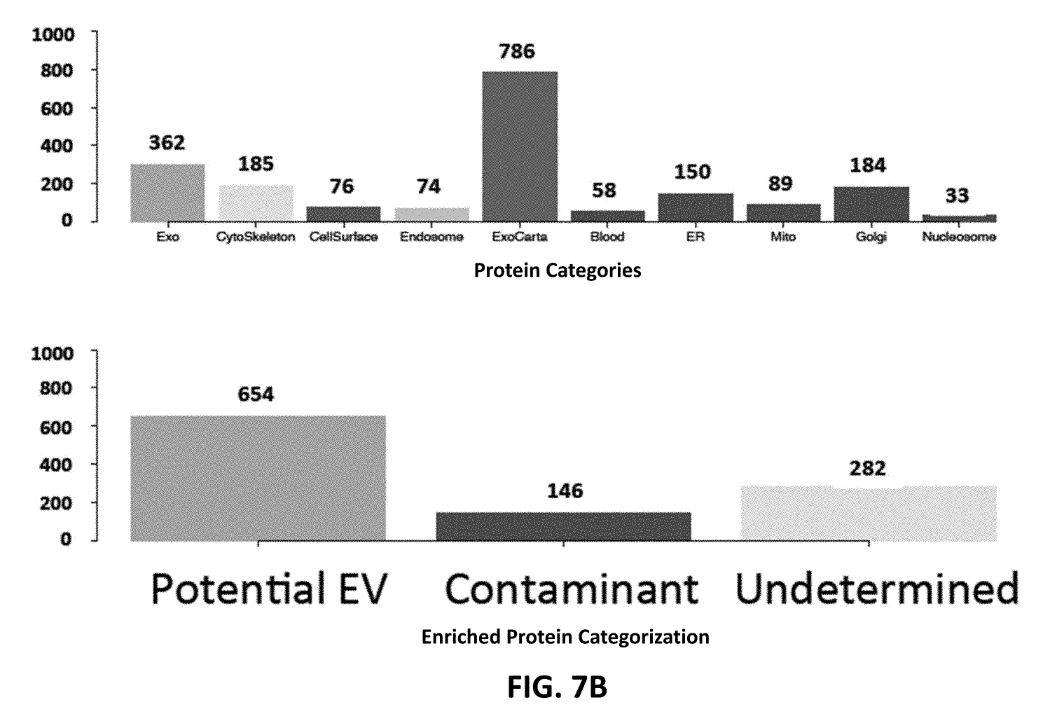

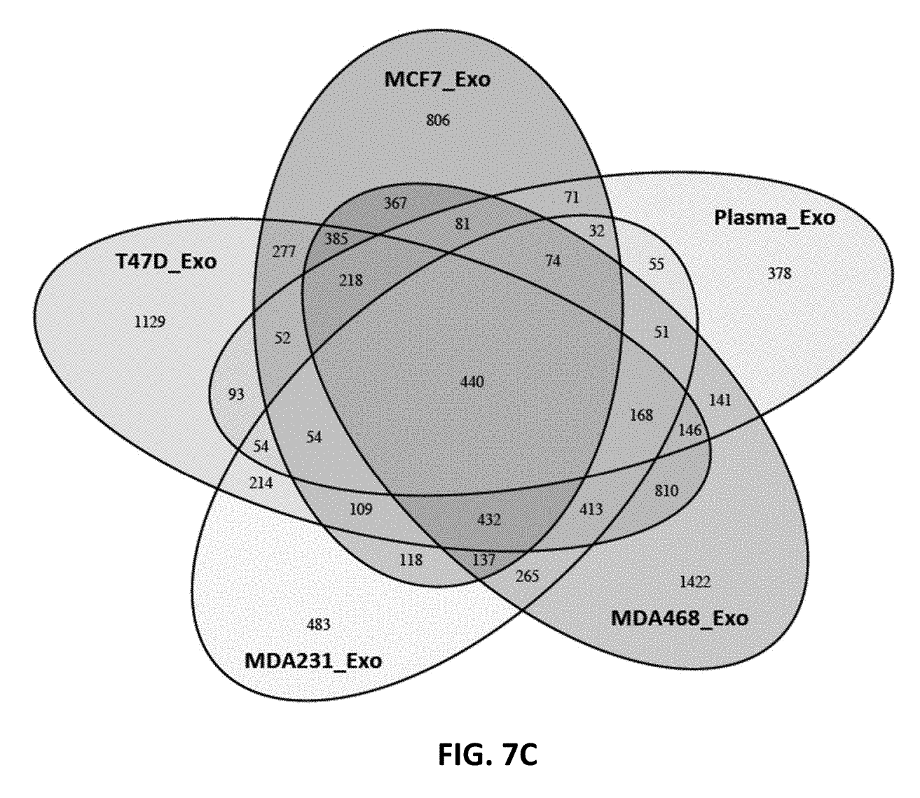

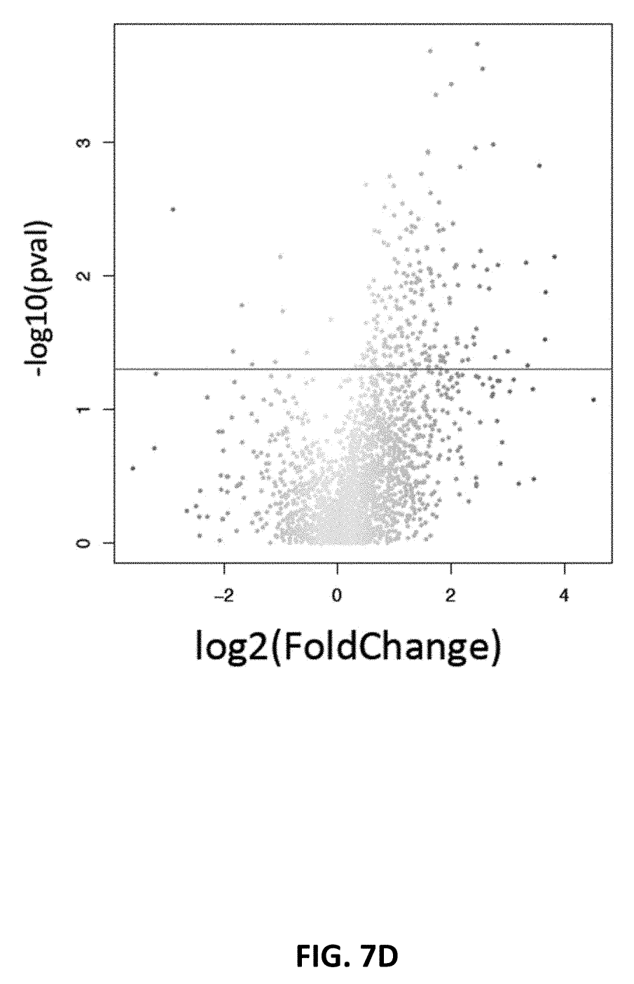

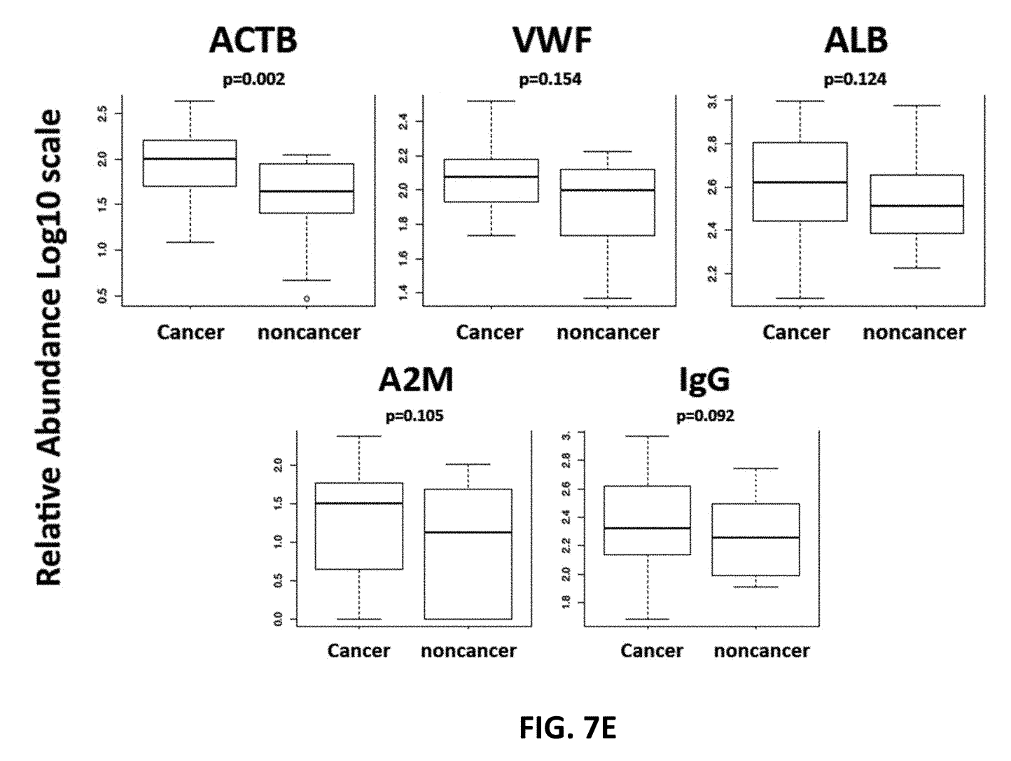

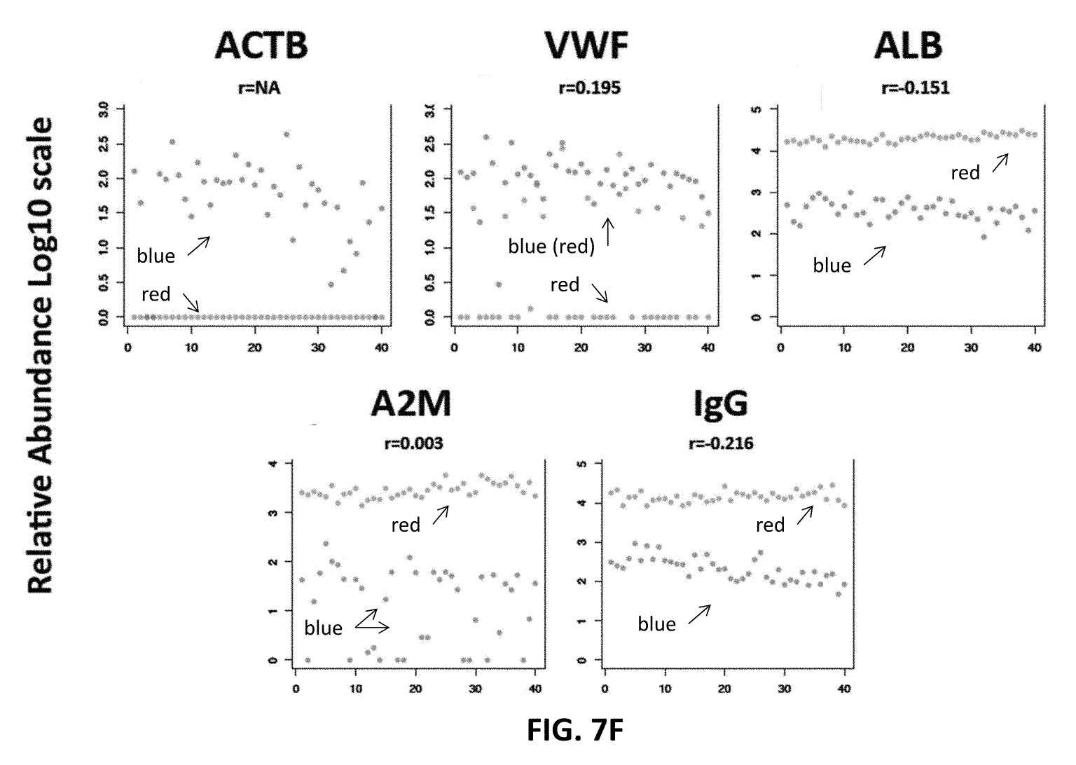

Breast cancer and non-cancer samples comprised age-matched samples (average of 55 years old) (FIG. 7A). Breast cancer samples were all from advanced clinical stage III and IV. Age and stage of the patients are listed in Table 5. Bio-specimens were obtained under an IRB-approved Biorepository Protocol. All subjects were consented with an IRB approved consent form and per 21 CFR 50.20 guidelines. Blood was collected using standard venipuncture to EDTA tubes and plasma was collected by standardized. Blood was spun in a Labofuge 200 for 10 minutes at 5300 RPM to remove cell debris. The clear layer of the plasma was aliquoted and transferred into cryovials and store at −80° C. Before EV enrichment described below, the plasma was quickly thawed in water at room temperature (RT) and centrifuged at 4000 g for another 15 min at RT to further remove any potential protein aggregation/cell debris. The plasma supernatant was used for the EV enrichment. Plasma aliquots for the EV enrichment below were all freeze-thawed for only one-time.

TABLE 5
Patient Characteristics

| Patient | Status | Stage | Stage M |
|---|---|---|---|
| 1 | Cancer | III | M0 |
| 2 | Cancer | III | M0 |
| 3 | Cancer | IV | M1 |
| 4 | Cancer | III | MX |
| 5 | Cancer | III | M0 |
| 6 | Cancer | III | M0 |
| 7 | Cancer | III | M0 |
| 8 | Cancer | III | M0 |
| 9 | Cancer | IV | M1 |
| 10 | Cancer | III | MX |
| 11 | Cancer | III | MX |
| 12 | Cancer | III | M0 |
| 13 | Cancer | III | MX |
| 14 | Cancer | III | M0 |
| 15 | Cancer | III | M0 |
| 16 | Cancer | III | M0 |
| 17 | Cancer | III | M0 |
| 18 | Cancer | III | M0 |
| 19 | Cancer | III | M0 |
| 20 | Cancer | III | M0 |
| 21 | Non-cancer | Unknown | Unknown |
| 22 | Non-cancer | Unknown | Unknown |
| 23 | Non-cancer | Unknown | Unknown |
| 24 | Non-cancer | Unknown | Unknown |
| 25 | Non-cancer | Unknown | Unknown |
| 26 | Non-cancer | Unknown | Unknown |
| 27 | Non-cancer | Unknown | Unknown |
| 28 | Non-cancer | Unknown | Unknown |
| 29 | Non-cancer | Unknown | Unknown |
| 30 | Non-cancer | Unknown | Unknown |
| 31 | Non-cancer | Unknown | Unknown |
| 32 | Non-cancer | Unknown | Unknown |
| 33 | Non-cancer | Unknown | Unknown |
| 34 | Non-cancer | Unknown | Unknown |
| 35 | Non-cancer | Unknown | Unknown |
| 36 | Non-cancer | Unknown | Unknown |
| 37 | Non-cancer | Unknown | Unknown |
| 38 | Non-cancer | Unknown | Unknown |
| 39 | Non-cancer | Unknown | Unknown |
| 40 | Non-cancer | Unknown | Unknown |

EV Enrichment from Plasma

Pluronic block copolymer F68 was from VWR Amresco in a powder format (VWR 95034-960; VWR International Ltd., Dublin, Ireland). The block copolymer was dissolved in phosphate buffered saline (PBS) to make a 15% stock solution. During use, the stock solution was further diluted in PBS such that when adding 100 μl of plasma to 900 μl of such solution, the final F68 concentration for enrichment is 2%. The mixture was incubated at room temperature for 30 min followed by centrifugation at 20000 g for 30 min at 4° C. The pellet was then washed with 1% F68 by vortexing following by centrifugation for 10 min at 4° C. at 20000 g. The supernatant was then removed and appropriate amount of PBS were added to re-suspend the fraction. For cell line EV spiked-in sample, 1 μg of the Vcap generated EV prepared as shown above was spiked into 100 μl plasma. Plasma EV extraction by Exoquick (System Biosciences, Inc., Palo Alto, Calif.) and Total Exosome Isolation kit (TEI, Thermo Fisher Scientific, Waltham, Mass.), instructions for 100 μl of plasma were followed. Protein quantification was performed by Micro BCA Protein Assay kit (Thermo Fisher Scientific) according to the instruction from the product.

DLS, ELISA and Western Blot

Particle size distributions was measured with DynaPro Plate Reader II (Wyatt Technology Corporation, Santa Barbara, Calif.), in three replicates, 5 acquisitions of 5 seconds each at 25° C. PBS buffer and UC-isolated cell line EV were used as control. CD9 ELISA was performed as follow: 1 μg of the protein from neat plasma (i.e., unmixed plasma), plasma with cell line EV (Vcap) precipitated by F68 and plasma alone precipitated by F68 were coated on the 96 well ELISA plate overnight at 4° C. The plates were blocked by 1% BSA in PBS for 1 hour. Biotinylated anti-CD9 monoclonal antibody (Abcam, ab34161; Cambridge, UK) were added as detection antibody followed by streptavidin-HRP; 100 μl of Ultra ELISA Substrate (Thermo Scientific Pierce Protein Biology) were used and OD₄₅₀ were recorded by the BioTek plate reader. For Western blot, protein samples were separated on 4-12% Bis Tris protein gel and transferred to a PDVF membrane. Primary antibodies included mouse anti-CD9 (Abcam), mouse anti-β actin antibody (Abcam), mouse anti-HSP70 antibody (Abcam), rabbit anti-Von Willebrand Factor antibody (Abcam), rabbit anti-human serum albumin antibody (Abcam), mouse anti-alpha2 macroglobulin antibody (Abcam) and rabbit anti human IgG antibody (Abcam). Chemiluminescent signals were captured by imager (PXi, Syngene USA; Frederick, Md.).

RNA Analysis, RNA Sequencing, Small RNA (smRNA) Sequencing and miR Assay

To extract total RNA and small RNAs (smRNA), Trizol LS reagent was used (Thermo Fisher). RNA, including total RNA and smRNA was purified through the miRNeasy column (Qiagen; Venlo, Netherlands) according to the instruction from manufactory. All samples were eluted in 20 μl of RNase-free water. 1 μl of these samples were used for bioanalyzer analysis (Agilent Technologies; Santa Clara, Calif.). Density analysis of the small RNA bands was performed in ImageJ software package [15]. The SMARTer Stranded Total RNA-Seq Kit (Clontech Laboratories; Mountain View, Calif.) was used to prepare RNA for Next Gen Sequencing (NGS). RNA was sequenced on the Illumina Miseq instrument according to the instruction from the manufacturer (Illumina, Inc.; San Diego, Calif.). The data were analyzed by a customized Tophat/cufflinks pipeline [16] against the Ensemble-GRCh38 human genome and further categorization according to their biotypes from Ensemble GRCh38.85 RNA annotation. For smRNA sequencing, the library preparation was performed according to the instruction of Clean-Tag smRNA kit from Trilink Biotech (San Diego, Calif.). Data was analyzed using a customized smRNA pipeline. Reads were normalized to RPM (reads per million mapped read) for comparison. smRNA recovery wasalso examined by let-7a Taqman probe assay (ThermoFisher) using the same amount of eluted sample. When comparing the recovery rate of let-7a from cell line EV-spiked plasma and cell line EV alone, Ct was normalized to the input sample column (all samples were processed the same way, eluted by same volume). To compare enrichment of let-7a from plasma enriched EVs to plasma alone, relative abundance was analyzed by ΔΔCt method with miR-451a as reference miR (one of the most abundant miR found in plasma [17]).

Transmission Electron Microscopy (TEM)

TEM and immuno-TEM analysis of EVs were performed as described previously [4] with modifications. In more detail, vesicle were re-suspended in 1×PBS and deposited onto a formvar coated slide of copper mesh EM grids (FF300-Cu-50, Electron Microscopy Sciences; Hatfield, Pa.). The vesicle-coated grids were washed, stained with 1% UO₂(CH₃COO)₂ and then viewed by the TEM using a Philips CM12 with Gatan model 791 camera. For the immuno-gold labelling with antibodies, blocked grids coated with EVs were briefly fixed by ice-cold 1:1 Ethanol/Methanol for 5 min followed by washing with PBS. The grids were transferred to a drop of the anti-CD9 antibody (Abcam) in PBS with 1% BSA, 0.01% Tween20 and incubated for 30 min. The grids were then washed, incubated with gold-labeled secondary antibody (Sigma-Aldrich; St. Louis, Mo.). The grids were stained with 0.5% UO₂(CH₃COO)₂ and then imaged by TEM.

Flow Cytometry Analysis

Isolated EVs were labelled with a cocktail of mouse anti-CD9-PE (BD Biosciences; San Jose, Calif.), anti-CD63-PE (BD Biosciences) and mouse anti-CD81 PE (BD Biosciences) or Mouse IgG1 kappa PE as isotype antibody control (eBioscience|Thermo Fisher Scientific). PBS, antibody isotype control, anti-tetraspanin cocktail diluent as well as samples without staining were processed as controls. PE fluorescence intensity associated with particle events was measured by flow cytometry (A50-Micro, Apogee Flow Systems; Hertfordshire, UK); Data analysis was performed in R3.2.2 environment with customized script, detail provided in Supplementary Methods.

Shotgun Proteomics

In-solution trypsin digestion was performed according to the manufactory instruction (ThermoFisher). Samples were analyzed by nanoflow reverse phase liquid chromatography using a Dionex Ultimate 3000 RSLCnano System (ThermoFisher) coupled in-line to a Q Exactive HF mass spectrometer (ThermoFisher). The nano LC system included an Acclaim PepMap 100 C18 5 μm 100A 300 μm×5 mm trap column and an EASY-Spray C18 2 μm 100A 50 μm×150 mm analytical column (ThermoFisher). Peptide samples were eluted with a two-step gradient of 2% to 30% B in 28 min then 30% to 45% B in 5 min, where B consisted of acetonitrile containing 0.1% formic acid. Blank samples consisting of 0.1% formic acid in water were injected between each sample and eluted with the same gradient profile and times as the samples. The LC system was interfaced with the mass spectrometry using an EASY-Spray electrospray ion source (ThermoFisher) and the samples were analyzed using positive ion spray voltage set to 2 kV, S-lens RF level at 65, and heated capillary at 285° C. The Q Exactive HF was operated in the data-dependent acquisition mode for fragmentation. MS1 survey scans (m/z 400-1400) were acquired in the Orbitrap analyzer with a resolution of 120,000 at m/z 200, an accumulation target of 3×10⁶, and maximum fill time of 50 ms. MS2 scans were collected using a resolution of 30,000 at m/z 200, an accumulation target of 1×105, and maximum fill time of 100 ms, with an isolation window of 1.5 m/z, normalized collision energy of 28, and charged state recognition between 2 and 7.

ProteoWizard [18] was used for peak-picking, filtering out peaks with intensity less the 100 and converting the file to mzML format. Protein search and identifications were performed using MS-GF+ [19] search engine on Homo sapiens (Uniprot TaxID=9606). For semi-quantification analysis, algorithm was establish according to the previous normalized spectra index (SIN) algorithm [20, 21] with modifications: sum of Intensity from each peptide MS2 scan were normalized by the parent peptide charge, such normalized intensity for the same peptide from different MS2 scans were then sum together; sum of the normalized intensity for the same peptide is then further normalized by the experimental molecular weight of the peptide, as a unit of normalized intensity per Dalton (mass). Normalized intensity per mass between different peptides of the same protein detected in the same sample was then averaged to obtain the final spectrum index (SI). For relative abundance, the SI was further normalized by the average total MS2 intensity among all samples.

Protein Categorization

To categorize the proteins for their relevance to EVs, individual GO terms were query for GO definition of extracellular exosome (0070062), cytoskeleton (0005856), cell surface receptor (0007166), endosome (0005768), Golgi apparatus (0044431), blood microparticles (0072562), endoplasmic reticulum (0044432), mitochondrion (0044429) and nucleosome (0000788). The ExoCarta database version 5 was obtained from www.exocarta.org. For the second categorization, proteins that were identified with GO terms of extracellular exosome, cytoskeleton, cell surface receptor, endosome as well as those in the ExoCarta database were regarded as “Potential EV” proteins; proteins with GO terms of blood microparticles, endoplasmic reticulum, mitochondrion as well as nucleosome were regarded as “Potential Contaminant.” Proteins that matched both potential EV and potential contaminant terms were regarded as “Undetermined.”

Statistical Models

Statistical modeling was performed using the R 3.2.2 environment. Leave-one-out (LOO) cross-validation model was built based on random forest. Briefly, to predict each test sample (1), the rest of the samples (39) were used as training set to build the model. In order to select the most important features to maximizing the removal of trivial features, a second layer of LOO models was built by within the 39 training set samples. In the second layer LOO models, features occurs in at least 90% of the individual second layer models were extracted and used to build the final prediction model for that training set, this model (from 39 samples) were then used to predict the test sample. The predicted probabilities for individual samples from the LOO cross-validation model were then used to plot the AUC curve. The final set of selected features were then collected by combining all the features retained in all the individual training models for the un-supervised cluster Heatmap.

Permutation predictions were performed by permuting the cancer/non-cancer sample label 10,000 times and the models as described above was applied to calculate the AUC for each permutation. Statistically significance p value was calculated by the percentage of permutation AUC that are higher or equal to the AUC performance from the correct label.

Other statistical analysis, including mean, standard deviation (SD), t test, and graphing, were calculated or performed in Excel or the R environment.

Results

Pluronic Block Copolymer F68 as a Reagent to Enrich EVs from Plasma

To examine whether the pluronic copolymer could be used as a reagent to enrich EVs from plasma, we first examined if the enriched fraction contains EV size particles. Plasma was precipitated by 2% of F68. The same protocol was also applied to UC purified cell line EVs diluted with or without plasma as positive controls. Dynamic Light Scatter (DLS) and transmission electron microscopy (TEM) was used to determine the size distributions and morphology of the recovered particles, respectively.

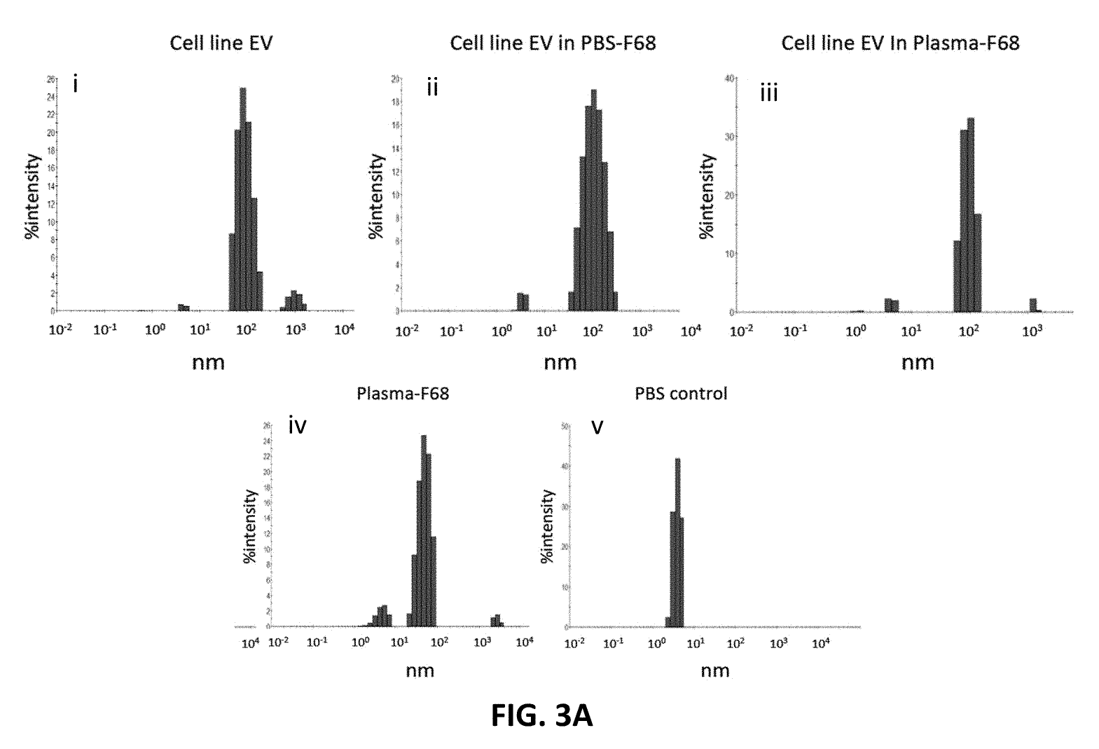

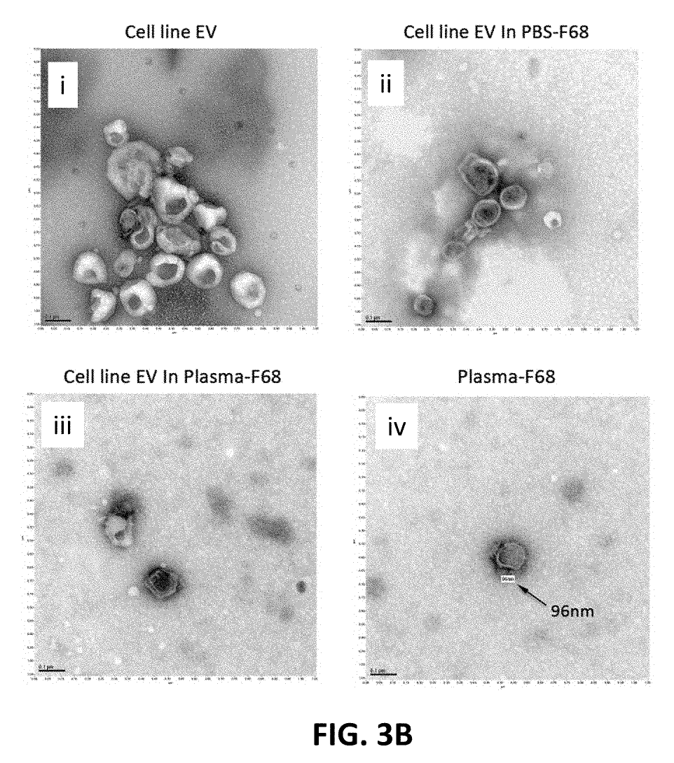

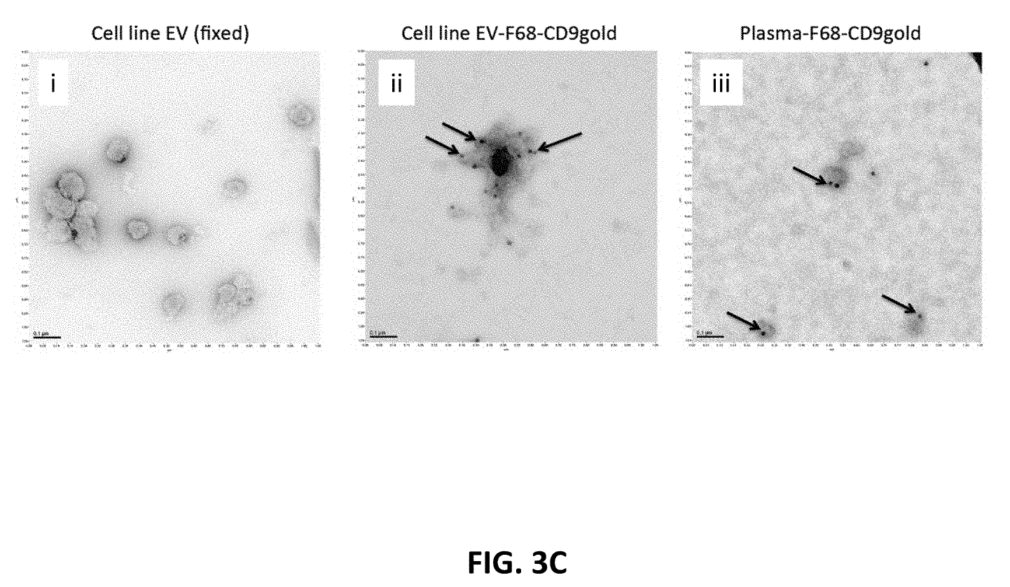

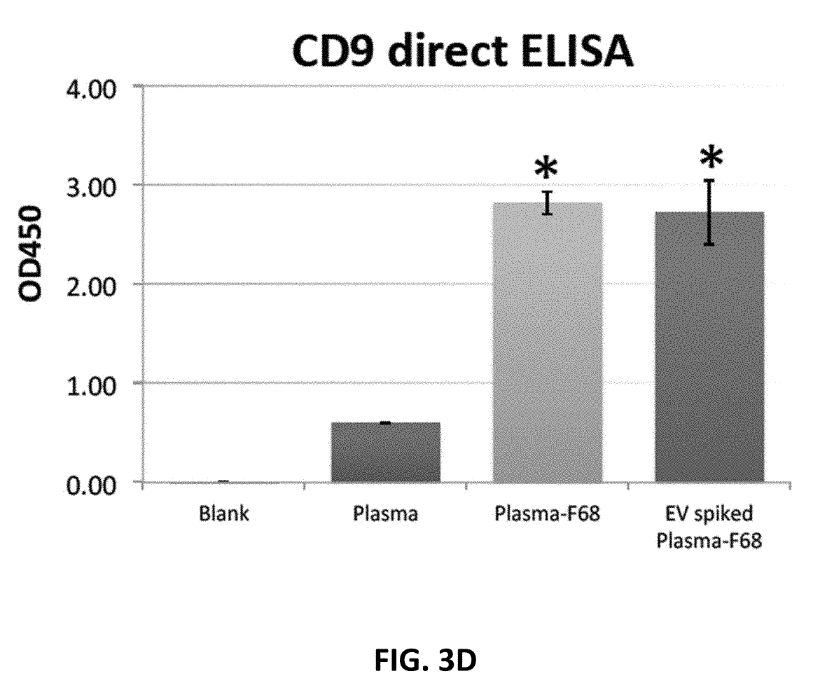

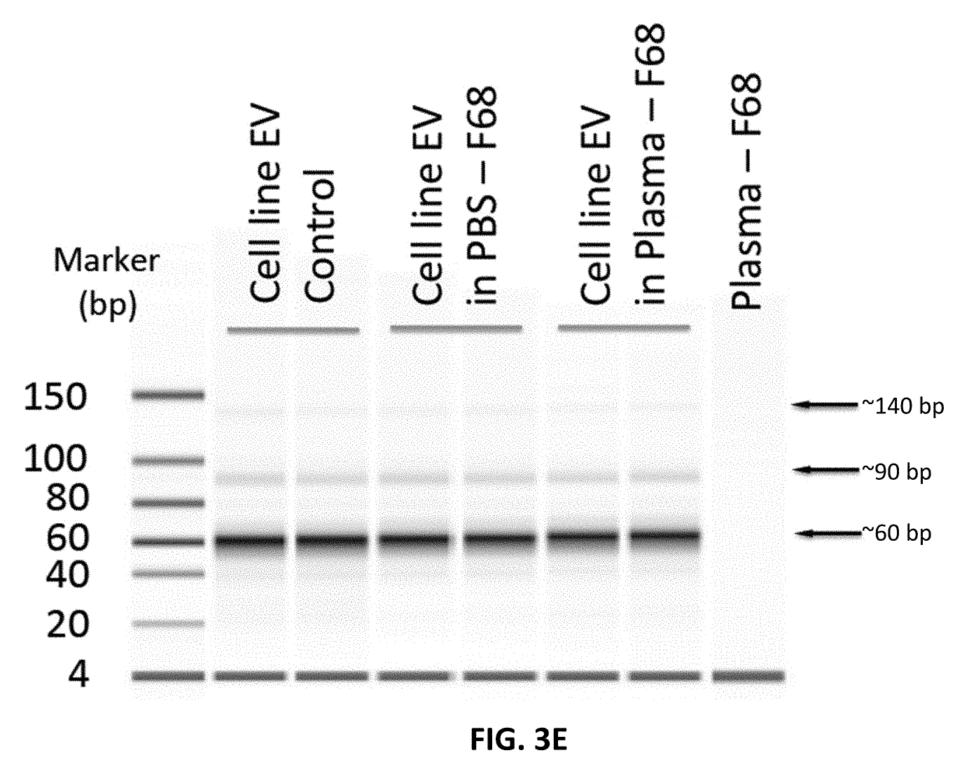

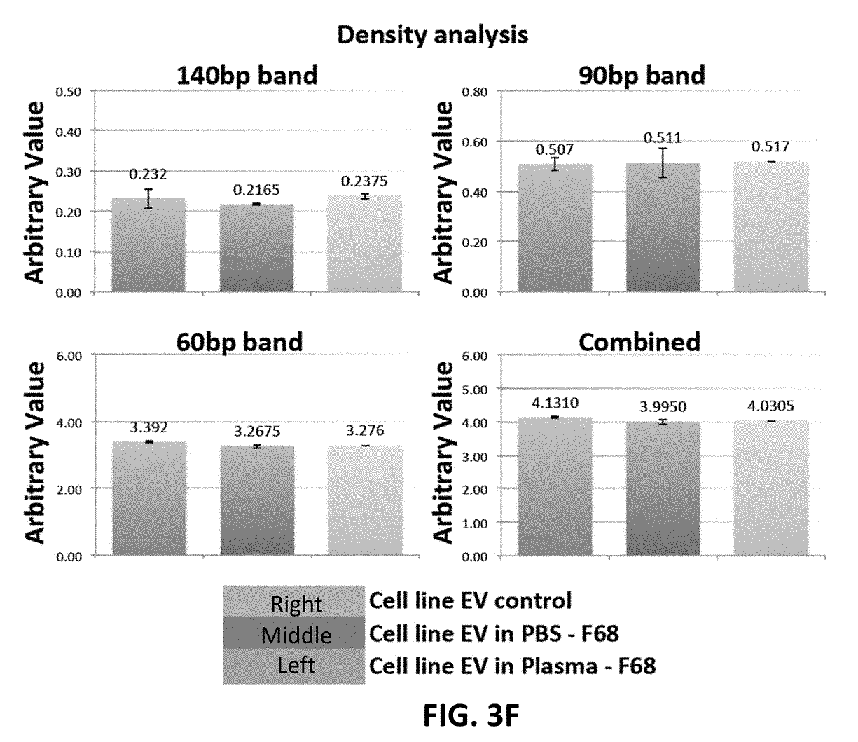

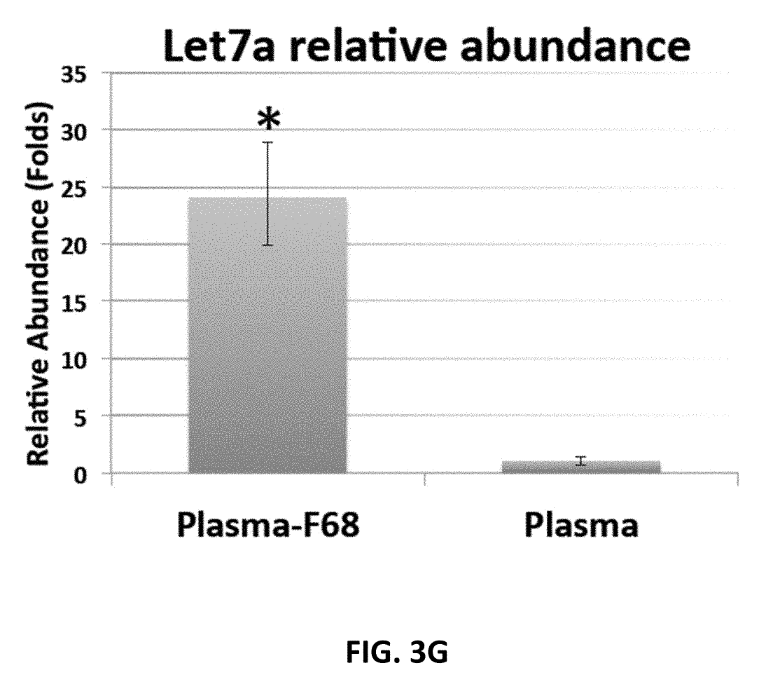

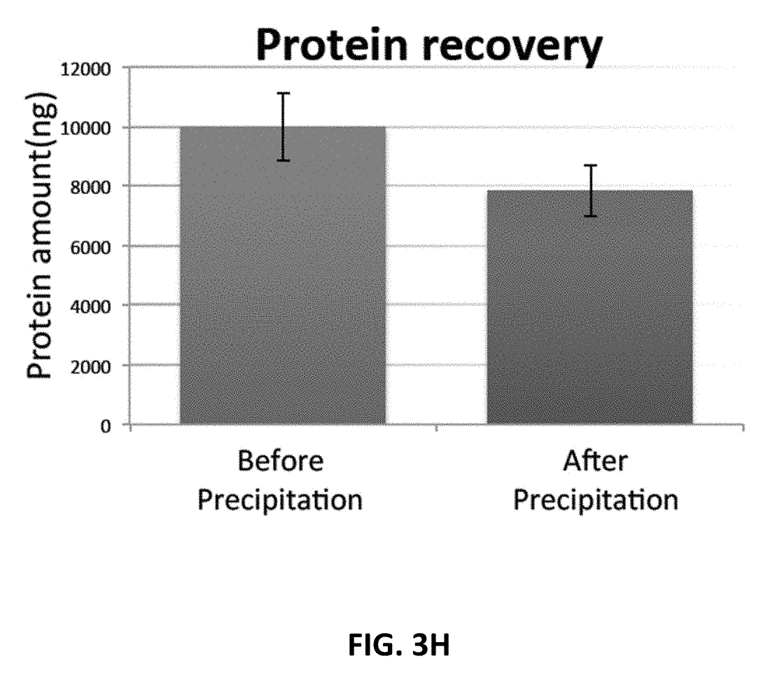

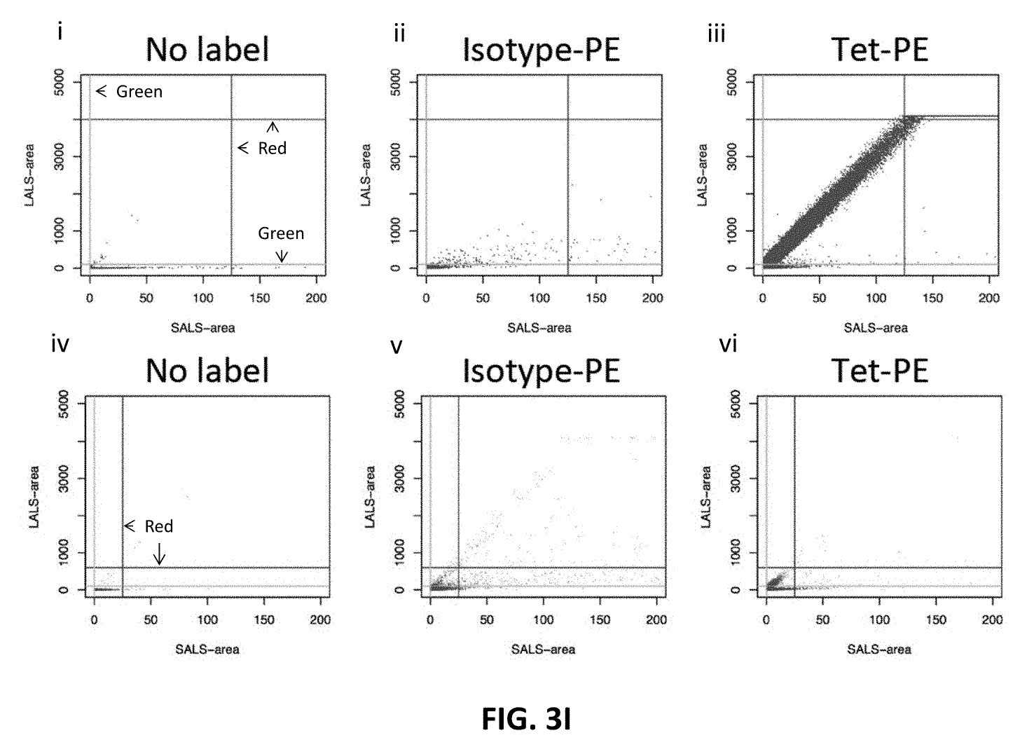

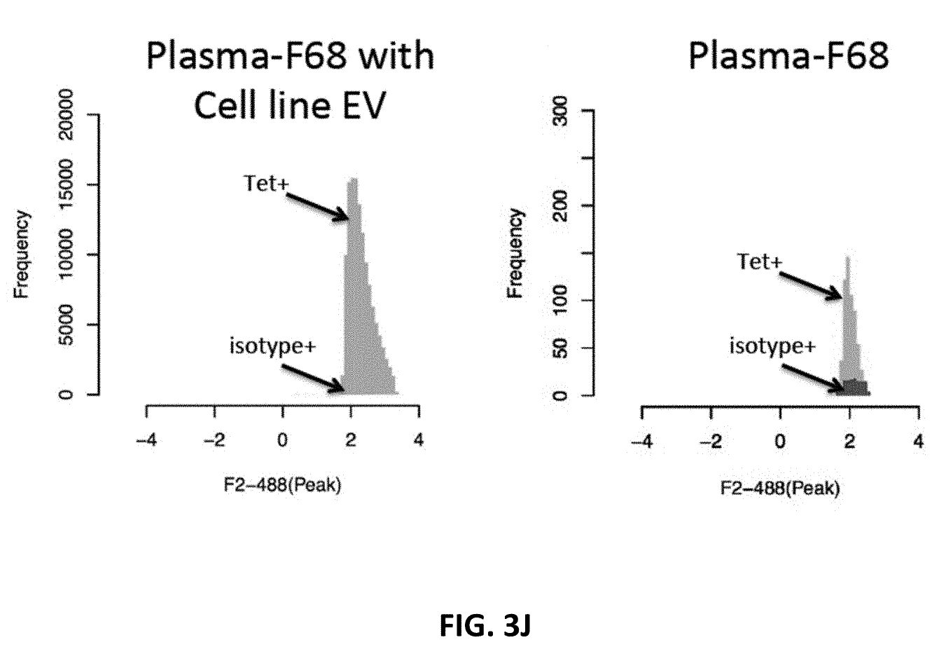

As shown in FIG. 3Ai, cell line EVs purified by UC ranged between about 30 nm to 300 nm, centered around 100 nm, which accords with EV sizes reported previously [2]. Distribution in the cell line EVs precipitated by F68 in the absence or presence of plasma showed a similar pattern to the cell line EV alone (compare FIG. 3ii-iii to FIG. 3Ai). Size distributions for plasma EV enriched by F68 ranged between 20 nm-100 nm (FIG. 3Aiv), which corresponds to the size of EV described previously [2]. In contrast, the PBS buffer control only showed particles below 10 nm (FIG. 3Av), far below the expected EV size range, indicating the particles in the buffer solution would not interfere with the analysis. We also observed small subpopulations of particles with a size distribution around ˜1000 nm in both cell line EVs enriched by UC and the plasma particles precipitated by F68 (FIG. 3Ai-iv, values in ii were too low to be seen in the plot). Without being bound by theory, this small portion of this subpopulation may consist of particles made up of microvesicles, apoptotic bodies, or from potential EV aggregates.

Transmission electron microscopy (TEM) was used to capture morphological detail of the enriched particles. UC purified cell line EVs, alone or recovered by F68 precipitation (in presence or absence of plasma) showed intact round shape vesicles with membrane structure (FIG. 3Bi-iii), similar to the shape of EV as described in previous studies [2]. The native plasma particles recovered by F68 precipitation also show similar shape with membrane structure and size (around 100 nm) (FIG. 3iv), which is consistent with the DLS results above.

To determine whether the enriched particles carried tetraspanins, a common EV indicator, enriched plasma EVs were labeled with immune-gold by anti-CD9 antibody [22]. For these scans, a modified protocol with ethanol/methanol fixation procedure was adopted. As a result, the shapes of EVs appeared to be more condensed and with lighter contrast compared to the regular protocol above. TEM scans showed cell line EVs alone (FIG. 3Ci) or precipitated by F68 were labeled with gold particles against the tetraspanin CD9 (FIG. 3Cii), indicating the presence of CD9 on their surface. Native plasma EVs enriched by F68 were also able to be labeled with anti-CD9 gold particles (FIG. 3Ciii), indicating the tetraspanin CD9 was also associated with the native plasma EVs enriched by F68.

The enriched plasma EV fraction recovered by F68 was further examined by a direct CD9 ELISA as shown in FIG. 3D. The cell-line EV-spiked plasma, after enrichment by F68, showed significantly higher signals compared to the same amount of plasma protein without enrichment (p<0.05). Similarly, plasma EVs enriched by the copolymer also showed significantly higher CD9 signals compared to the plasma control (p<0.05). These results further indicate that the EV marker CD9 was enriched in the F68 precipitated fraction.

The efficiency of recovery of small RNA species from the EVs precipitated from plasma samples was compared with that of cell line generated EVs. Three distinct smRNA patterns (˜140 bp, ˜90 bp and ˜60 bp) could be detected from Vcap cell line generated EVs (FIG. 3E). Such smRNA signature could be used to evaluate the recovery rate of EVs by densometry analysis. Without being bound by theory, we hypothesized that if the precipitate reagent is harmful to the EVs, for example disrupting the membrane structure, internal material including the RNAs, could be degraded or removed during the washes and reflecting on the intensities of the smRNA signature bands. As shown in FIG. 3E, the patterns and intensity of the cell line EV in PBS precipitated by F68 are comparable to the direct RNA extraction from same amount of cell line EVs. EV enriched from plasma sample with cell line EVs by F68 also show similar smRNA pattern as the cell line EV alone. These results indicate that the F68 enrichment did not result in substantial loss (if any) of EV payload smRNAs.

Densometry analysis was performed for all three individual smRNA bands to further evaluate the cell line EV smRNA recovery rate by F68 precipitation. Results are summarized in FIG. 3F. As shown, no significant difference was observed from analyzing respective smRNA bands between cell line EV, cell line EV precipitated by F68 and cell line EV in plasma precipitated by F68 samples (p>0.05), nor by the combined density of all three signature bands between those three samples (p>0.05). The recovery rate of each individual band for cell line EVs in PBS precipitated with F68 ranged from 93% to 100% with final combined rate of 97% in reference to the cell line EV control. Recovery rate of each individual band for cell line EVs in plasma precipitated with F68 also ranged from 97% to 102% with final combined rate of 98%.

MicroRNA species let-7a has been reported as payload inside EVs [23, 24, 25] and is abundant in Vcap cell line generated EVs. We examined the let-7a recovery rate from Vcap EVs by qPCR. No significant difference was observed in let-7a level between cell-line EV spiked plasma precipitated by F68 to same amount of cell line EV alone (Ct 22.3±0.08 vs Ct 22.8±0.06, p>0.05), indicating the level of let-7a recovered by F68 was comparable to the level from same amount of original cell line EVs, consistent to the smRNA analysis that show high recovery rate of EV smRNA from the F68 enrichment.

Let-7a level from native plasma EVs precipitated by F68 were also compared to the let-7a level from whole plasma extraction. There was no significant difference in recovered let-7a level in the plasma EVs enriched by F68 compared to same amount of plasma RNA extraction (Ct 31.6±0.3 vs Ct 31.4±0.7, p>0.05), indicating the precipitation recovered most of the circulating plasma let-7a microRNAs. Relative enrichment of let-7a in comparison to the original plasma was also evaluated by AACt analysis by normalizing let-7a levels to miR-451a, an abundant plasma miR [17]. As show in FIG. 3G, let-7a level in the plasma EVs fraction precipitated by F68 (left bar) enriched more than 20-fold than from the corresponding neat plasma (right bar), which may be due to reduction in recovery of plasma miR-451a (Ct 26.4±0.5 vs Ct 22±0.7, p<0.05). These results indicate that EV related let-7a was enriched by the F68 precipitation, whereas plasma related miR was significantly reduced. Taken together, these results suggest that efficient recovery of microRNA is plasma EVs could be obtained by the copolymer precipitation while avoiding substantial contamination of non-EV microRNA species.

In addition, EV recovery efficiency was analyzed in term of protein recovery rate. Cell line EVs in PBS were precipitated by F68 according to the above protocol. Protein before and after precipitation were measured. As shown in FIG. 3H, about 80% of the cell line EV proteins (before: total protein amount is 9991±1156 ng, after: 7852±852 ng, 4 replicates for each sample) were recovered after the precipitation. The result further supports the results above demonstrating >90% recovery of EVs by smRNA densometry analysis and let-7a qPCR analysis.

Flow cytometry (FL) analysis was used to further verify the EV identity of the particles recovered from F68 precipitation and compatibility of the resulting sample to downstream applications. EVs enriched by F68 precipitation were labeled with a fluorescence labeled anti-tetraspanin (CD9/CD63/CD81) antibody cocktail. FIG. 3Ii-iii shows FL analysis of the EVs enriched by F68 from plasma sample with cell line EVs. When the enriched particles were not labeled, the majority of the particles distributed horizontally along the SALS-area axis with narrow LALS-area range (FIG. 3Ii). We observed a similar pattern for the same particles labeled with isotype antibody control (FIG. 3Iii). However, when the particles were labeled with the anti-tetraspanin antibody cocktail, a dense population appeared on the plot (FIG. 3Iiii). In addition, a population appeared on the horizontal that was distinct from the noise particles found in sample labeled with isotype antibody as well as non-labeled particles.

Native plasma EVs enriched by F68 copolymer were also analyzed by the same FL scheme (FIG. 3Iiv-vi) with similar results to the cell line EVs. Ntive EVs enriched from plasma, when labeled with tetraspanin antibody, show two distinct populations (FIG. 3Iiv), one aligned to the distribution from non-staining controls (FIG. 3Iv) as well as the sample stained with isotype antibody control (FIG. 3Ivi), and the other population show signal range similar to the positive control in FIG. 3Iiii.

Based on such finding on the light scatter plot, minimal and maximal threshold were determined manually (FIG. 3Iiv-vi, green and red lines, two-step gating as described in Zhong et al., JOURNAL OF EXTRACELLULAR VESICLES, 2018, VOL. 7, 1458574, which reference is incorporate herein by reference in its entirety) to select the particles events for fluorescence signal analysis. The selected populations were summarized in distribution histogram according to their PE fluorescence signals. EVs enriched from plasma sample with cell-line EVs showed that majority of the selected populations were tetraspanin positive (lighter shading) while the none-specific events that associated with isotype-antibody-PE labeling in the selected population was minimal (darker shading, almost not visible) (FIG. 3J, left). In the native plasma EVs enriched by F68, similar pattern was also shown with more tetraspanin positive events compared to the non-specific signal from isotype-PE labeling (FIG. 3J, right), while the PE signal range in the selected population are very similar to that from cell line EV spiked plasma positive control showed in FIG. 3J, left. This result further confirms that there is a subpopulation from native plasma EVs enriched by F68 could be identified with similar tetraspanin expression pattern compared to the cell line generated EVs. Further details of the FL analysis are provided in Zhong et al.

Proteomic Profiling of Plasma EVs Enriched by Pluronic Copolymer

Our results have shown that the plasma particles enriched by F68 precipitation were EV-size membrane structures, contained RNA/miRNA, and the majority displayed the EV marker tetraspanin proteins. Although tetraspanins (CD9/CD63/CD81) are frequently used as EV markers, they are not necessarily universal markers for all EV populations from all varieties of cell types [22], e.g., including all EVs shed into plasma. To evaluate potential EV-related proteins that could be detected and enriched by the copolymer method, shotgun semi-quantitative mass spectrometry (MS) analysis was performed. In addition, in order to evaluate the purity of the plasma EV recovered, two commercially available PEG-based EV enrichment methods (Exoquick and TEI) were also used as comparison.

EV fractions from each plasma sample were enriched from three different protocols: F68 as provided herein, and the commercially available Exoquick and TEI Kits. After enrichment according to the respective protocols, protein recoveries were measured and compared: total protein recovery was 0.46% for F68 protocol, 16% from Exoquick protocol and 4.6% for TEI kit. As >99% of the protein mass in plasma are from high abundant plasma proteins [26], higher yields may indicate contamination with highly abundant plasma proteins in addition to EVs.

Both high abundant plasma proteins (including serum albumin (ALB), Alpha-2-macroglobulin (A2M) and human plasma antibody (IgG) and the EV related proteins (including β-actin (ACTB), Heat shock 70 (HSP70) and von Willebrand factor (VWF)) were examined by semi-quantitative MS analysis. ALB and IgG are the two most abundant plasma proteins, and A2M is also one of the high ranking proteins in the plasma [26]. High amount of ALB not imply plasma protein contamination, and may reduce the sensitivity of the MS by narrowing its dynamic range. CD9, ACTB, HSP70 and VWF were used as indicators of plasma EV enrichment. ACTB is cytoskeleton protein whereas HSP70 is cytosolic proteins. Both have been reported to be enriched in EVs [27, 28, 29]. VWF has been reported to be one of the protein markers found in platelet derived EVs [28, 29], which accounts for 70%-90% of the EVs in the plasma. Without being bound by theory, protocols that could enrich plasma EV should likely to include this protein.

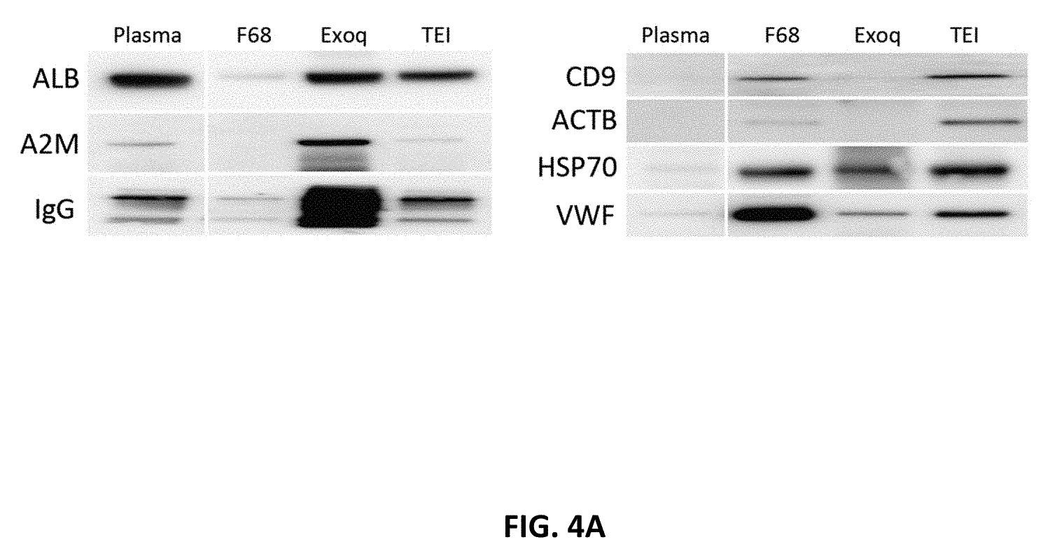

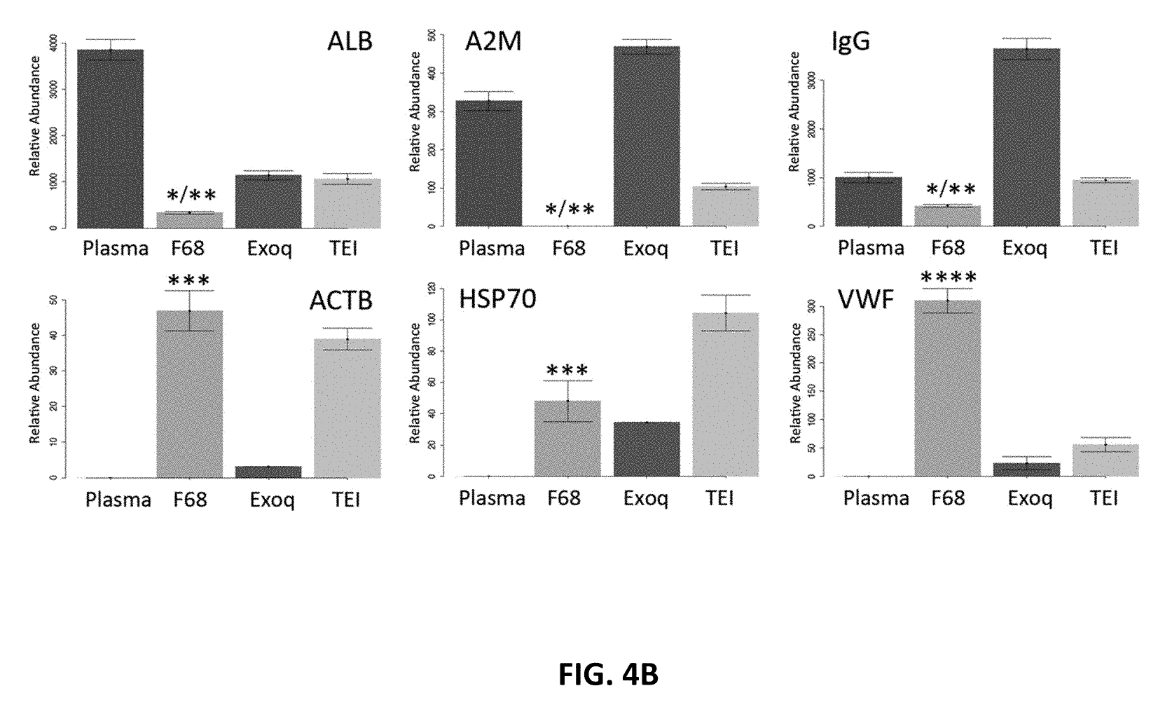

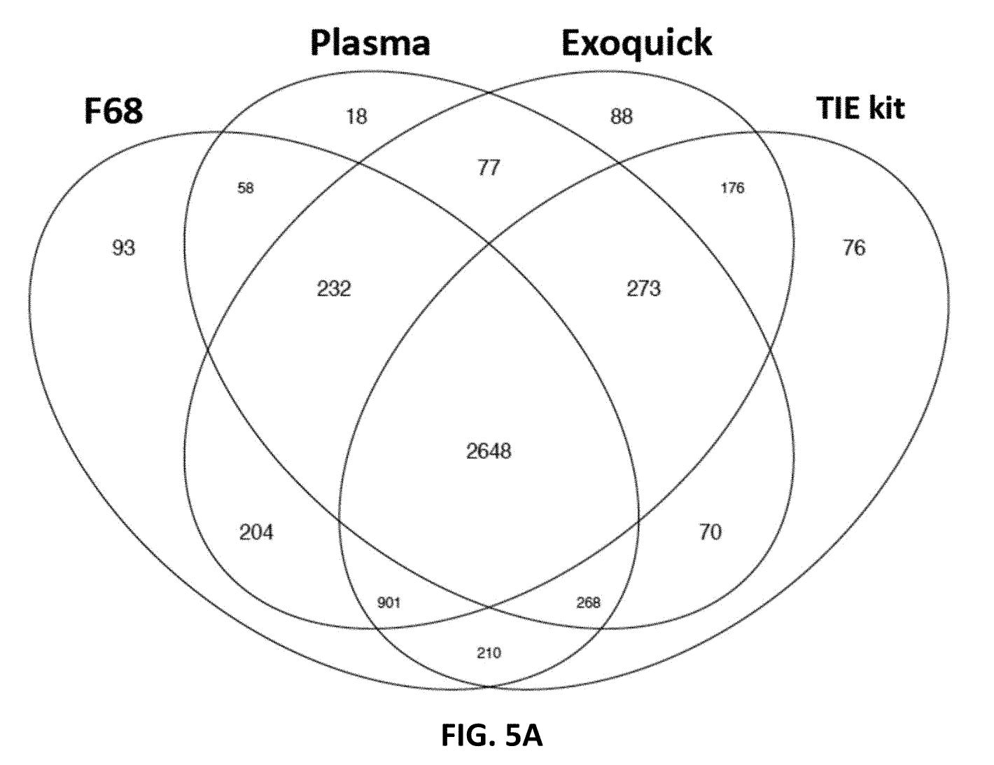

Western blots were first used to examine these protein indicators. See FIG. 4A. As shown in the figure, the relative abundance of ALB in the neat plasma was highest of all. The Exoquick and TEI kit samples retained high amounts of ALB. In contrast, the level of ALB in plasma EV enriched by F68 was greatly reduced. Similarly, EVs enriched by F68 methods consistently yielded the lowest contamination of A2M and IgG, whereas the Exoquick sample retained abundant A2M and IgG, while TEI kit showed slightly reduced in A2M but not IgG. On the other hand, EV markers CD9 and ACTB were only detected in plasma EVs enriched by F68 and TEI kit but not detected in neat plasma or EVs enriched by Exoquick. HSP70 was enriched in all three methods compared to the neat plasma. VWF was significantly enriched in plasma EVs enriched by F68, but less so in the samples enriched by Exoquick and TEI.

FIG. 4B shows semi-quantitative MS analysis of the same proteins as FIG. 4A. As expected, plasma contained the highest level of ALB. Plasma EVs enriched by Exoquick and TEI reduced ALB by about 70%, but both were still >400% higher than that from the F68 method provided herein. A2M protein was reduced by ˜65% in plasma EVs enriched by the TEI kit compared to neat plasma (p<0.05), whereas A2M was not detected in plasma EVs enriched by F68. In contrast, the Exoquick sample showed significant enrichment of A2M compared to others(p<0.05). Plasma enriched EVs by our F68 method significantly reduced the recovery of IgG proteins to about 35% compared to the neat plasma (p<0.05). Again, the plasma fraction enriched by Exoquick contained significantly more IgG than the other two methods (p<0.05). The EV marker ACTB was highest in plasma EV enriched with the F68 and TEI protocols, whereas the Exoquick sample yielded little ACTB. HSP70 was enriched by all three methods compared to neat plasma, with highest by TEI and comparable level enrichment between Exoquick and F68. Most significantly, VWF showed was highly enriched in the F68 EV fraction compared to others (p<0.05).

The results between Western blot (FIG. 4A) and the semi-quantitative MS analysis (FIG. 4B) were consistent. Though CD9 was readily detected on the Western blot, it and other tetraspanins were difficult to be detected in MS, which may be due to high hydrophobicity [30]. Nevertheless, the Western blot of CD9 confirmed enrichment of the tetraspanin protein in the plasma particles enriched by F68 precipitation, consistent to the result from the above ELISA, TEM and FL analysis.

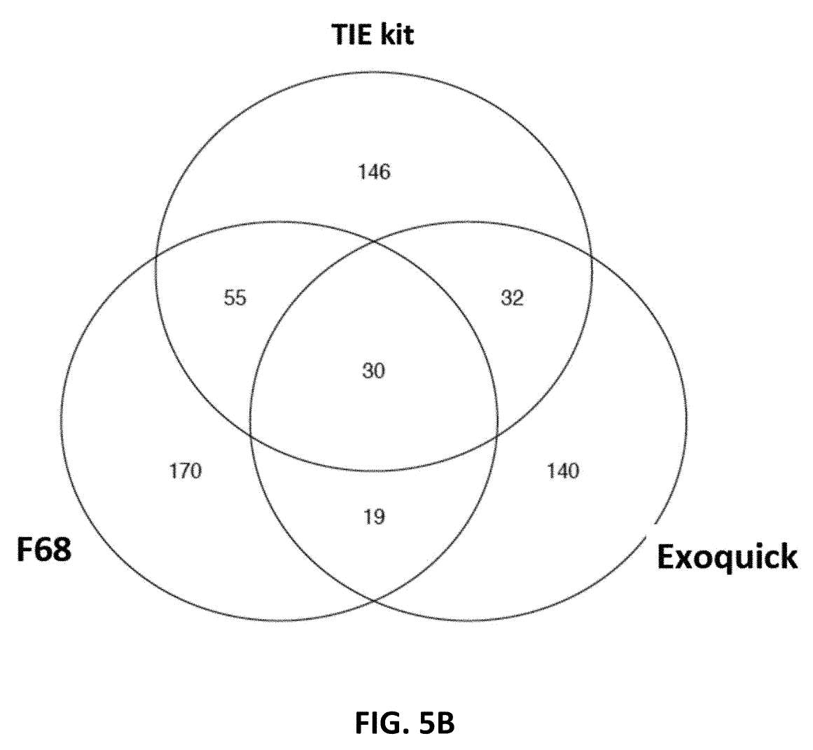

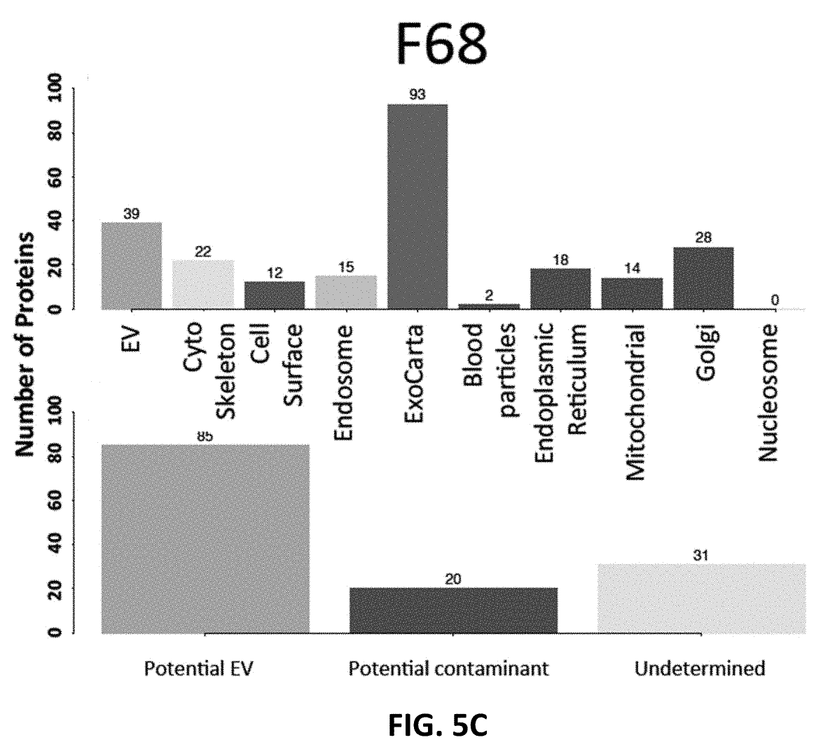

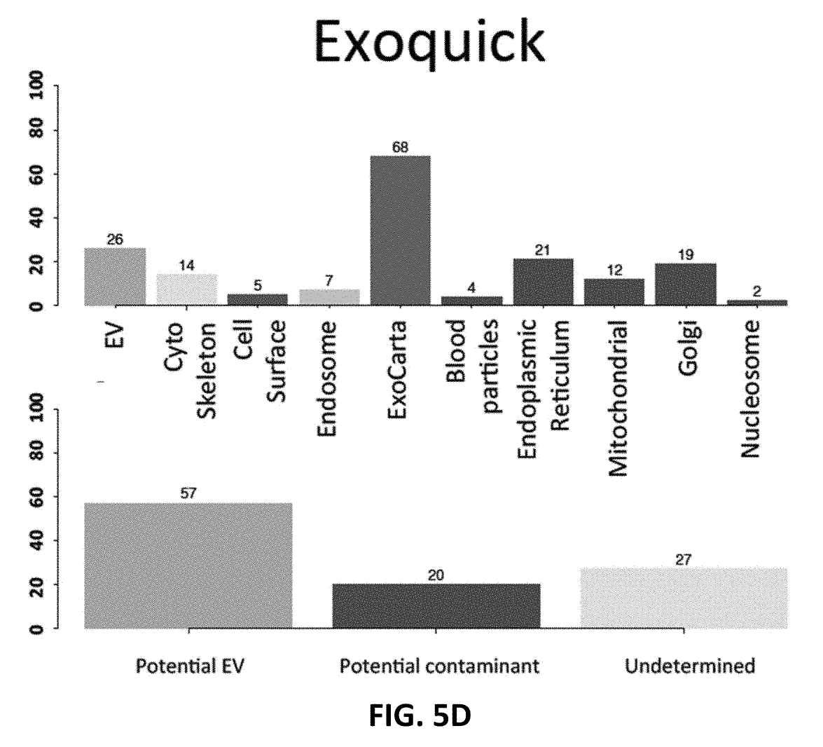

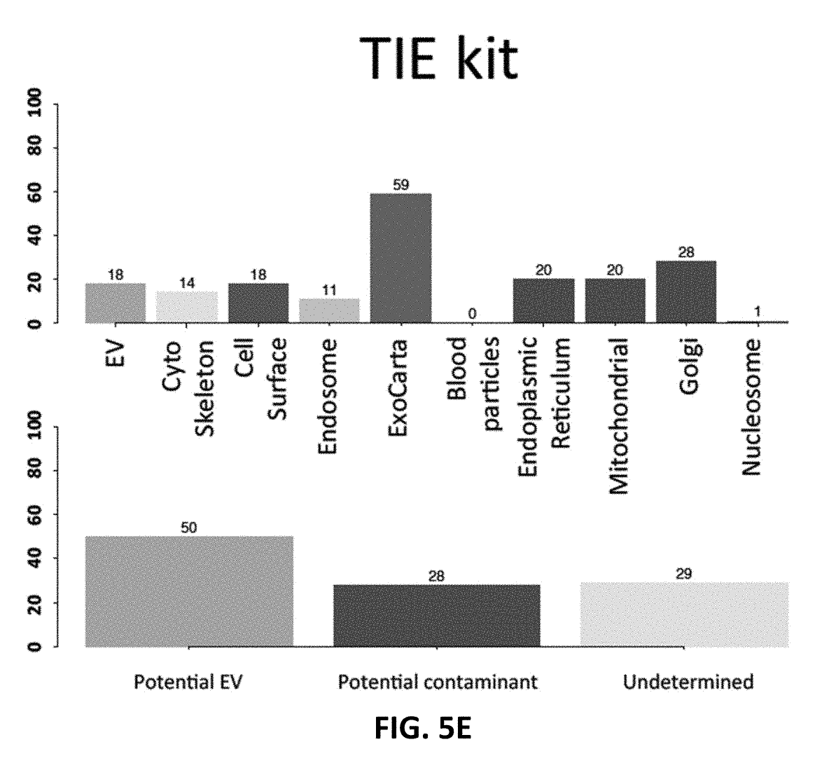

Shotgun proteomics analysis identified >5000 proteins from all three EV enrichment methods. In FIG. 5A, all detected features regardless of quantitation are accounted for in the Venn diagram. Some proteins were only identified in the sample enriched by each technique. Over half of those proteins (2648) were found in the samples enriched from all methods as well as in the corresponding plasma. 93 unique proteins were identified from EVs enriched by F68 compared to others, compared to 88 for Exoquick and 76 for TEI kit.

We further examined proteins that were enriched in the precipitates recovered by different enrichment methods. Features were first filtered by none-zero data points from individual methods as well as in the neat plasma. The average relative abundances of particular protein features from 4 technical replicates were used to determine fold change for each enrichment method over the neat plasma. T-tests were performed to calculate statistical significance. The highly enriched features were defined as the average relative abundance should be significantly higher (p<0.05 by t-test) than the corresponding neat plasma by equal or over 10 folds from respective methods. Therefore, sets of features with valid quantitative enrichment of equal or over 10 fold to neat plasma were obtained. These features from different methods were then used in the Venn diagram of FIG. 5B. Among the 592 shared protein features identified from all three EV enrichment methods with significant enrichment level from plasma, 170 are unique to F68 protocol, 140 proteins are unique to Exoquick protocol, and 146 proteins from TEI kit.

The identified proteins were then categorized according to their GO terms to explore cellular function. Proteins were first categorized by their association with extracellular exosome (Exo), cytoskeleton, cell surface, endosome, blood macromolecule, endoplasmic reticulum (ER), mitochondrial, Golgi apparatus and nucleosome (FIGS. 5C-E, upper). Cytoskeleton, cell surface and endosome related proteins are ubiquitous, and may be involved in the biogenesis of EV [22]. Proteins were also queried against the EV protein database ExoCarta [31]. These proteins were then categorized as “Potential EV” proteins. In contrast, blood macromolecules, ER, mitochondrial, Golgi and nucleosome related proteins are less likely to be present in EVs [22] due to their different biogenesis pathway. These were categorized as “Potential Contaminant.” Proteins not identified as “Potential EV” or “Potential contaminant” were regarded as “Undetermined” (FIGS. 5C-E, lower).

Among the highly enriched proteins from plasma EVs recovered by F68 (including the unique features for individual methods in both FIG. 5A and FIG. 5B), 40 proteins were identified as potential EV proteins, 22 as cytoskeleton proteins, 12 as cell surface proteins, 15 as endosome related proteins and 93 proteins were found matched in ExoCarta EV protein database (FIG. 5C, upper). When categorized only by the three groups (“Potential EV”, “Potential Contaminant”, and “Undetermined”), there were 85 EV-related proteins, 31 undetermined, and 20 potential contaminants (FIG. 5C, lower). The same analysis was applied to the samples from Exoquick and TEI kit preparations (FIGS. 5D-E). About 2-fold greater numbers of EV related proteins were found in the F68 samples than either Exoquick (40 to 26) or TEI kit (40 to 18). Similarly, the number of highly enriched “Potential EV” proteins in F68 protocol was about 50% more than corresponding Exoquick and TEI kit protocol (85 vs 57 and 60 respectively). Results above showed the F68 method significantly reduced the highly abundant plasma protein contamination in the enriched EVs fraction. Here, we further showed that the F68 enrichment method was able to enrich more “Potential EV” protein features compared to the other two precipitation-based protocol while greatly reducing contamination with the plasma high abundant protein.

NGS Profiling of Plasma EVs Enriched by Pluronic Copolymer

Let-7a qPCR above showed that the RNA inside plasma EVs enriched by F68 was well preserved. We performed total RNA sequencing and smRNA sequencing to further identify RNA species that exist in the plasma EVs enriched by F68.

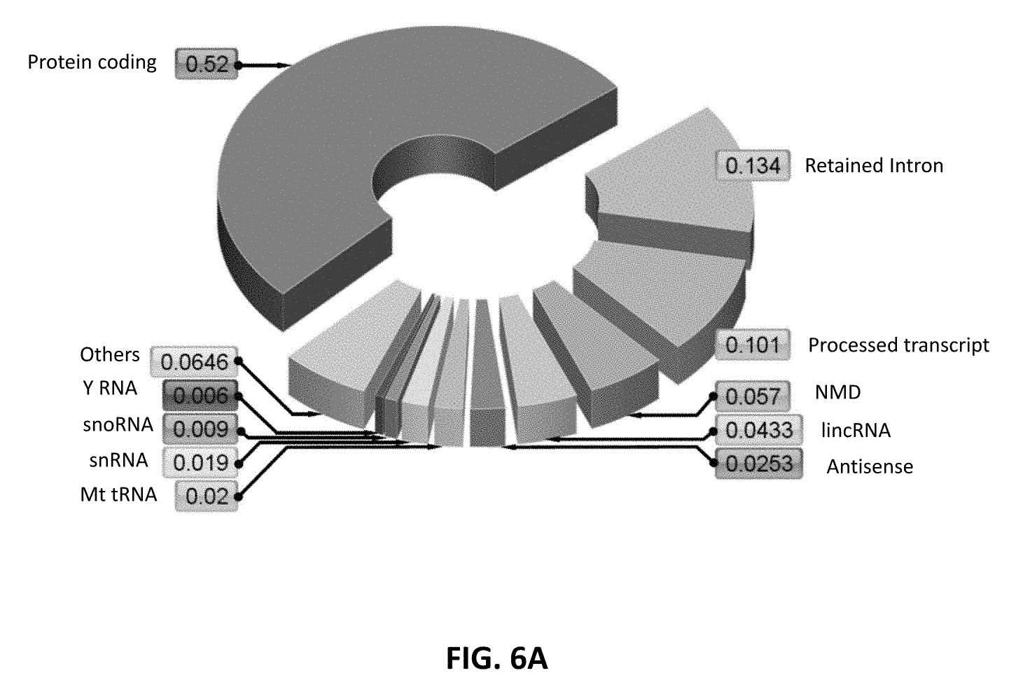

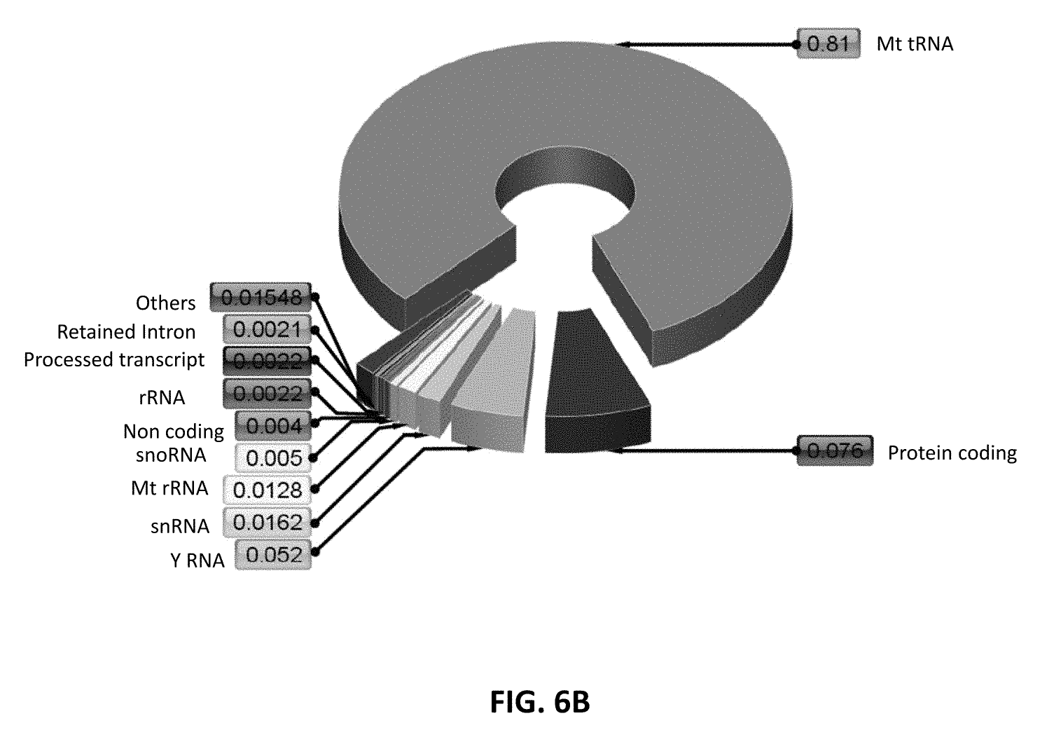

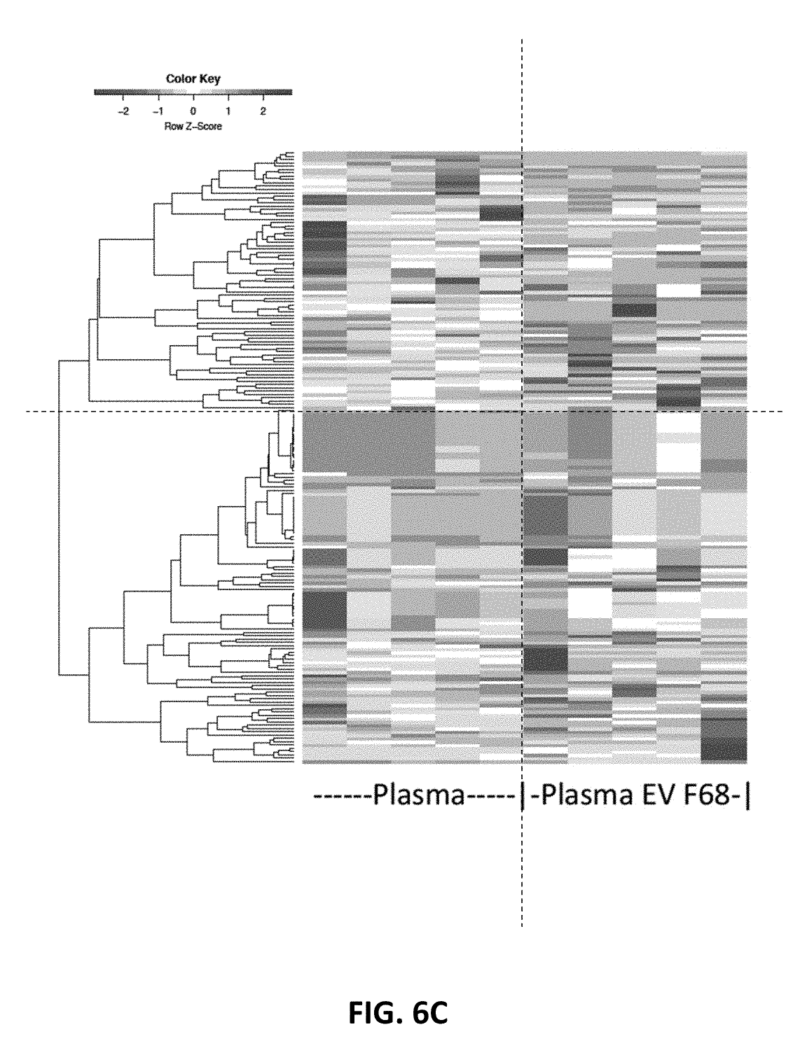

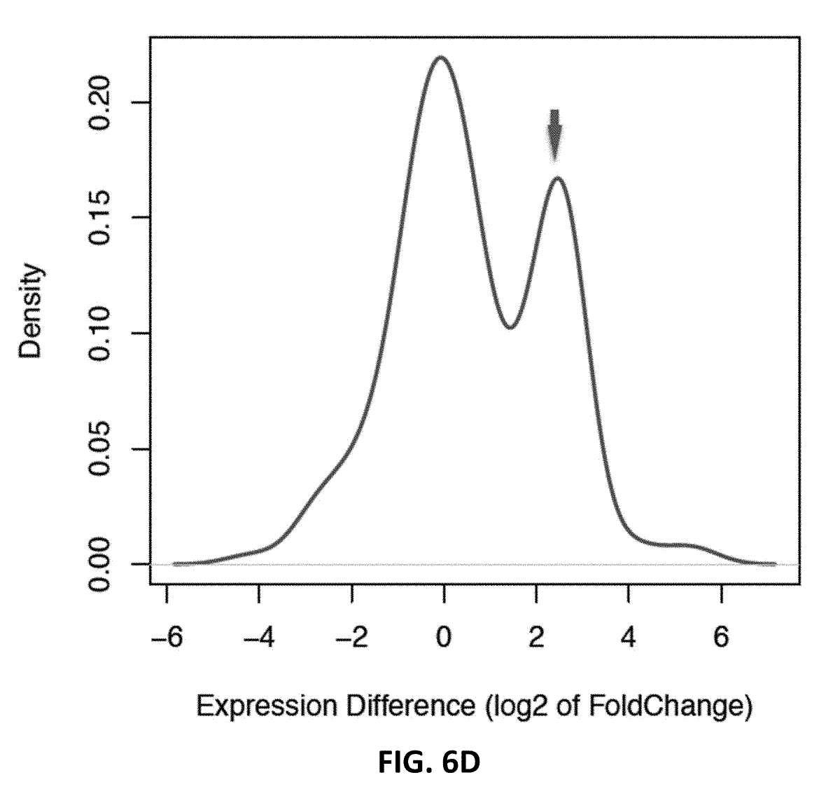

Total RNA profile is summarized in FIGS. 6A-D. The genes detected by NGS were categorized according to the biotype from Ensemble GRCh38.85 RNA annotation in terms of RNA species count (FIG. 6A) as well as relative abundance according to FPKM (Fragment Per Kilo base of transcript Per Million mapped reads, FIG. 6B). As shown in FIG. 6A, among all RNA species detected in the plasma EV enriched by F68, ˜58.5% are protein coding RNA species (including protein coding 52%, alternative splicing species(retained_intron) ˜6.5%), and about 5.7% of the transcripts match the non-sense mediated degradation category (NMD), consistent with a previous study showing RNA species subjected to RNA degradation could be found in EVs [32]. Other detected RNA species were different subtypes of non-coding RNAs, including 10% transcripts without valid ORF, 4.3% Long, intervening noncoding RNA (lincRNA), which also have been reported to be packaged into EVs [33, 34], and about 2% MtRNA, including mitochondria-origin tRNA and rRNA, which are also reported in previous researches[32]. Other non-coding RNA species, such as multiple antisense RNA, snRNA, snoRNA, Y_RNA that have been reported to be packaged into EVs [32, 33, 35] were also identified by NGS.

The relative abundance analysis (FIG. 6B) showed that Mt_tRNA was the most abundant RNA species from the plasma vesicle fraction precipitated by F68, accounting for more than 80% of the RNA, followed by protein_conding transcripts (7.6%). Though NMD RNA was identified above, their levels were relatively lower in the vesicle fraction, accounting for less than 0.08%. Y_RNA also had relative small amount of species number, but accounted for more than 5% abundance of the total RNAs, consistent to the previous report that considerable amount of Y_RNA was found in EVs [32].

Because the total RNA sequencing may not identify all short RNA fragments, smRNA sequencing was also performed by a ligation-based sequencing method [36], whose optimal templates are 5′ phosphorylated RNAs, such as miRNA and those smRNA derived from tRNA (tRFs) and Y_RNA (RNY) but act like miRNA [37, 38]. A variety of smRNA species were observed that mapped to miRNA, tRFs, RNY, piRNA and lincRNA with length range from 18 to 40 nucleotides. To evaluate if the smRNA profile could distinguish the enriched EVs from the corresponding plasma, a supervised-hierarchical clustering of the smRNA species was performed. See the heat map shown in FIG. 6C. Two smRNA clusters were formed. With respect to the dashed lines in the figure, the upper left quadrant was enriched in plasma compared to EVs (and vice versa), whereas the lower right quadrant was enriched in the F68 EVs compared to plasma (and vice versa). In addition, relative quantitative measurement showed a bimodal distribution of smRNA in reference to plasma, with one set of smRNA features enriched from the plasma (FIG. 6D).

Comparing Plasma EVs from Advanced Breast Cancer Non-Cancer Samples

The above results demonstrate that the pluronic copolymer method provided herein was able to enrich plasma EV with high efficiency with less contamination as compared to alternate methods. To further demonstrate use of the F68 method, plasma EVs from 20 breast cancers with advanced clinical stages (clinical stage III and IV) and 20 age-matched non-cancers were enriched using F68 precipitation. Semi-quantitative MS profiles were identified for each sample and compared to determine protein signatures related to breast cancer.

A total of 2108 proteins were detected in the cancer and non-cancer groups. Categorized by their respective GO term as described above, over 300 proteins related to EVs, 185 proteins related to cytoskeleton, 76 related to cell surface proteins, and 74 related to endosome. 786 were found in ExoCarta database (FIG. 7B, upper). Overall, 654 unique proteins were identified as potential EV protein features, 282 proteins as EV or free contaminant, and 146 proteins may be contamination from cellular organelles (FIG. 7B, lower). Over 4 times of the potential EV proteins were identified compared to the potential non-EV contaminant, which is consistent to result from the above methodology characterization.

Proteomic profiling was also performed on F68 enriched EVs from breast cancer cell-lines including MCF7, MDA-MB-468, MDA-MB-231 and T47D (FIG. 7C). 3653, 5550, 3099 and 4994 proteins were detected in the MCF7, MDA-MB-468, MDA-MB-231 and T47D cell lines generated EVs, respectively, versus the 2108 proteins detected in plasma as described above. 872 proteins were found in common all four cell line EVs.

The four breast cancer cell lines we used have each shown aggressive, metastasic and invasive behavior in varieties of studies. Without being bound by theory, we hypothesized that there would be common features related to such behavior. If such common factors exist in cell line EVs, they might also likely exist in the breast cancer cells from patients, and they might be detectable from the enriched plasma EVs. By comparing proteins from cell line EVs to the enriched plasma EVs from patients, 440 proteins was identified as common features between EVs generated by all four aggressive breast cancer cell lines and the enriched plasma EVs from patients. These data suggest that these 440 features were associated with breast cancer EVs in the plasma.

FIG. 7D shows a comparison between cancer and non-cancer groups of the 2108 protein features detected in the patient samples. 217 features show statistically significant differences between cancer and non-cancer groups (p<0.05). 197 features had relative abundance significantly higher in the cancer group compared to non-cancer group by greater than or equal to 1.5 fold (p<0.05), whereas 6 of features show relative abundance significantly lower in the cancer group compared to non-cancer group by equal or less than 0.5 fold (p<0.05).

Proteins found in plasma EVs and high abundant plasma proteins were compared between the cancer and non-cancer groups (FIG. 7E), as well as between plasma EV fraction and the neat plasma (FIG. 7F). The EV marker ACTB showed statistically significant different levels between cancer and non-cancer groups (FIG. 7E, p<0.05). In contrast, VWF showed no difference (FIG. 7E, p>0.05). Similarly, no statistically significant differences were observed between cancer and non-cancer group for abundant plasma proteins ALB, A2M and IgG (FIG. 7E, p>0.05). FIG. 7F shows that the EV markers ACTB and VWF were both significantly higher in the enriched EV fraction compared to the corresponding neat plasma (p<0.05). In contrast, highly abundant plasma proteins (ALB, A2M and IgG) showed magnitudes higher in neat plasma sample compared to the corresponding enriched EV fraction, consistent to the above result that the EVs from the patient plasma were enriched by the F68 copolymer (p<0.05). There was, however, little correlation in the high abundant plasma proteins in the EV fractions with those from the corresponding neat plasma.

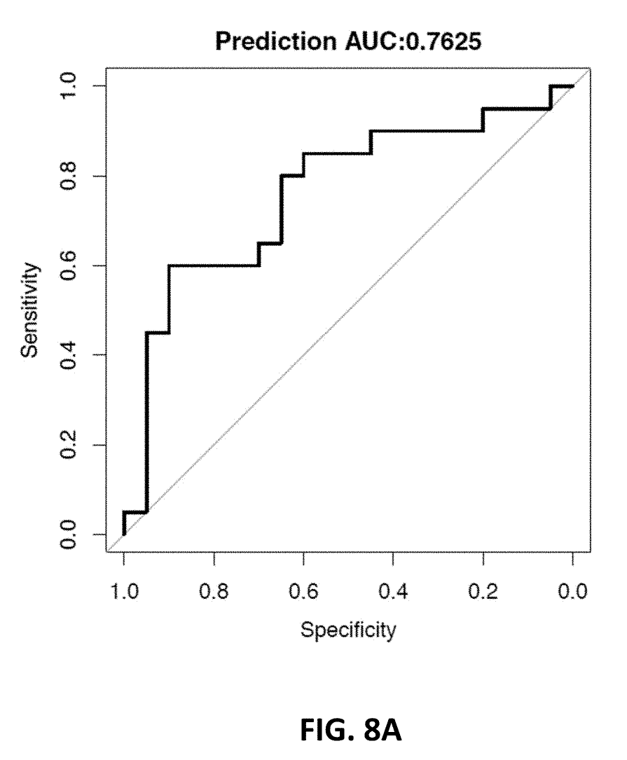

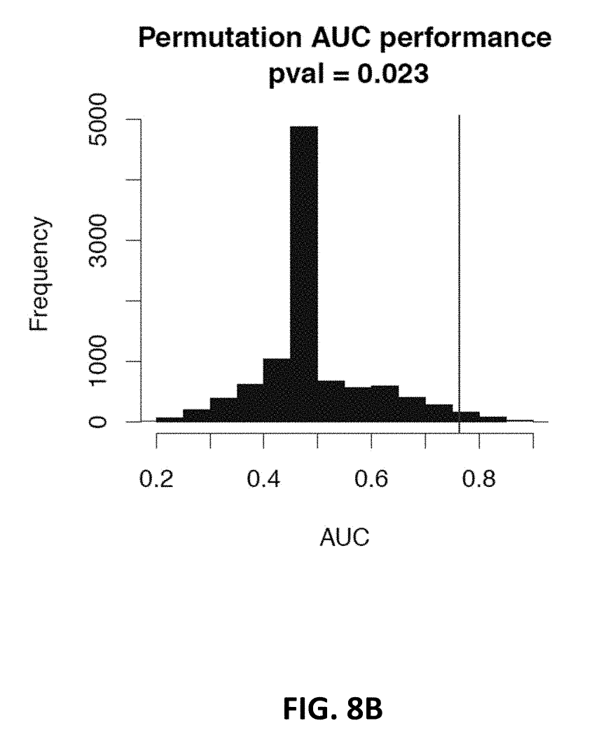

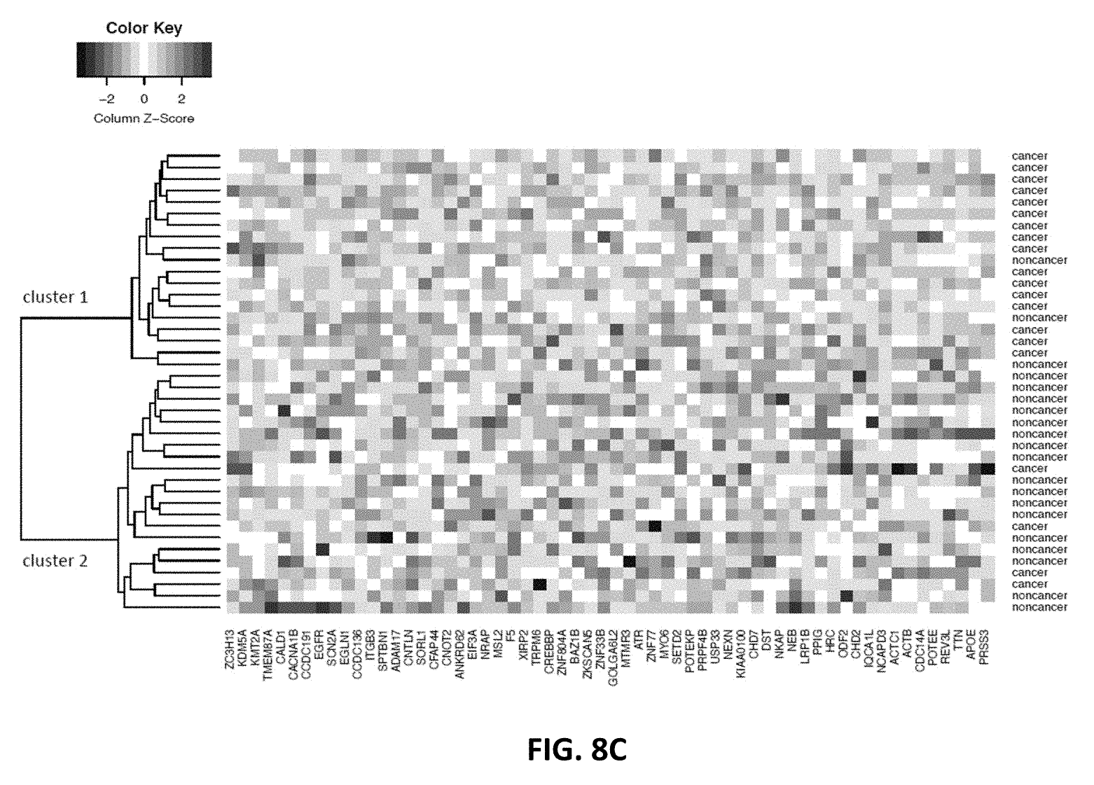

In order to assess the potential classification power by the plasma EV proteomic profiles, a leave-one-out (LOO) cross-validation model was built. The prediction performance in term of the area under curve (AUC) value was obtained. FIG. 8A show the overall AUC performance was 0.7625. One optimal point on the line had a specificity of 0.9 and sensitivity about 0.6. To examine whether such performance was due to a random chance, AUC was calculated from 10000 random permutations of the sample label. The distribution of AUC performance from the permutations is shown on FIG. 8B. Compared to the permutation AUC performance, the AUC 0.7625 obtained from the correct sample permutation is statistically significant (FIG. 8B, p<0.05), indicating the classification performance between the original cancer/non-cancer group was not due to a random chance. Rather, these data suggest that the protein features from enriched plasma EVs were capable of separating the advanced breast cancer patient from non-cancer controls.

60 features that were consistently contributed to the model in differentiating cancer from non-cancer sample were selected. See FIG. 8C and Table 6. An un-supervised hierarchical clustering was performed to classify the current patient samples. As shown in FIG. 8C, two clusters were formed, one with mostly cancer sample (“cluster 1,” 16 cancers vs 3 non-cancers) and the other is mostly non-cancer (“cluster 2,” 17 non-cancers vs 4 cancers). Thus the clustering separated the sample with sensitivity of 80% and specificity of 85%, further indicating that the above feature selection process was able to screen features that separates advanced cancer patients from non-cancer controls.

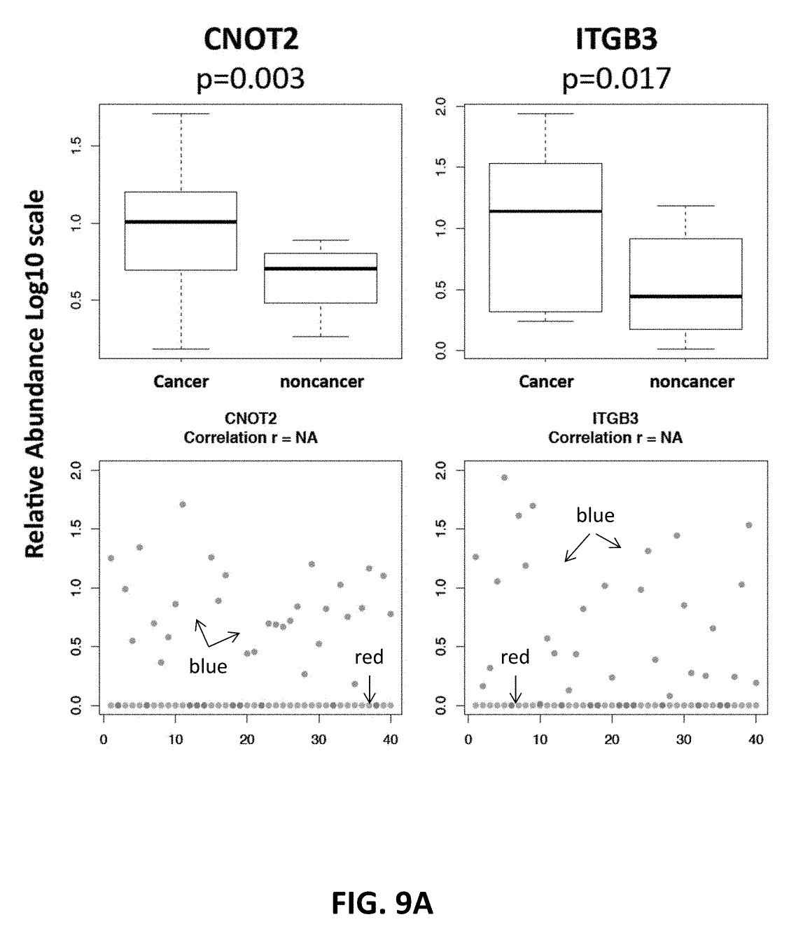

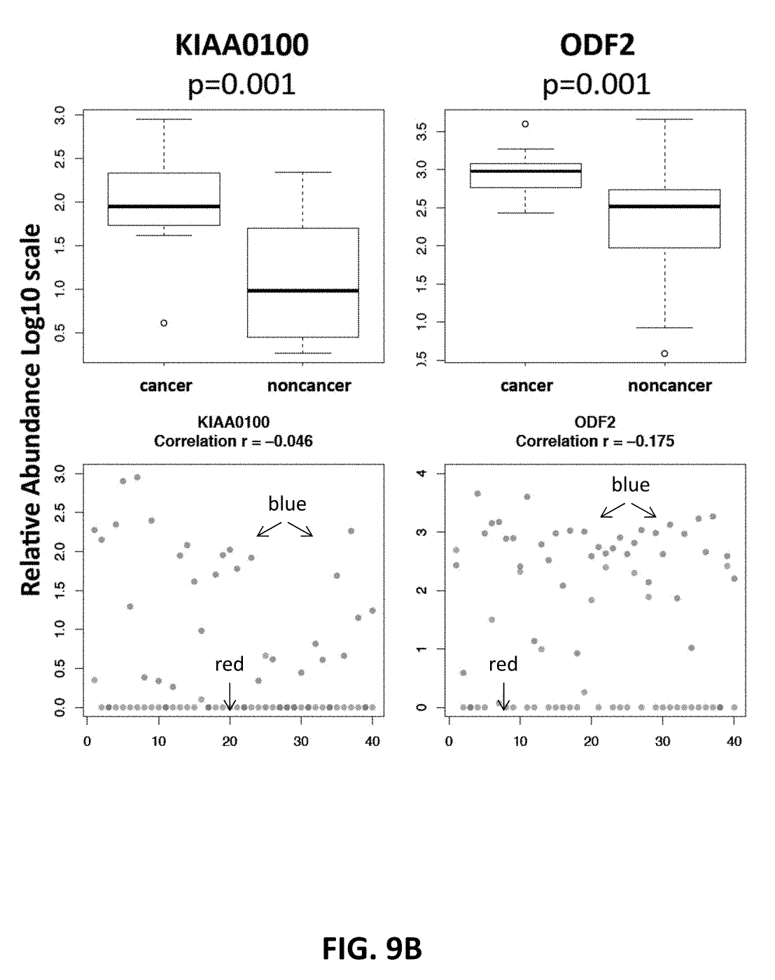

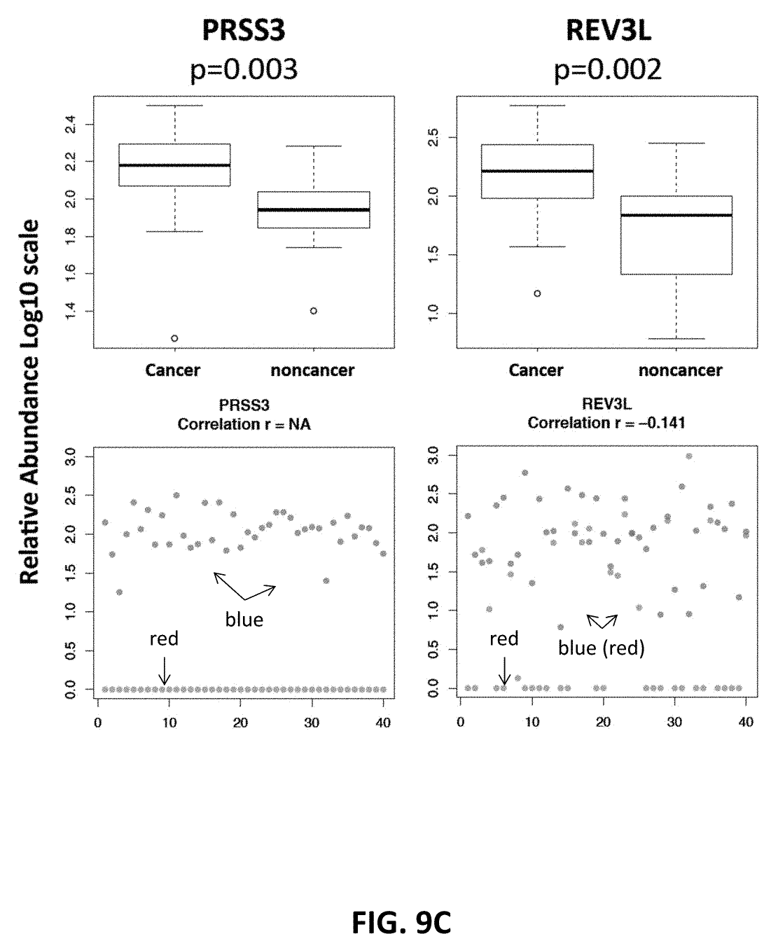

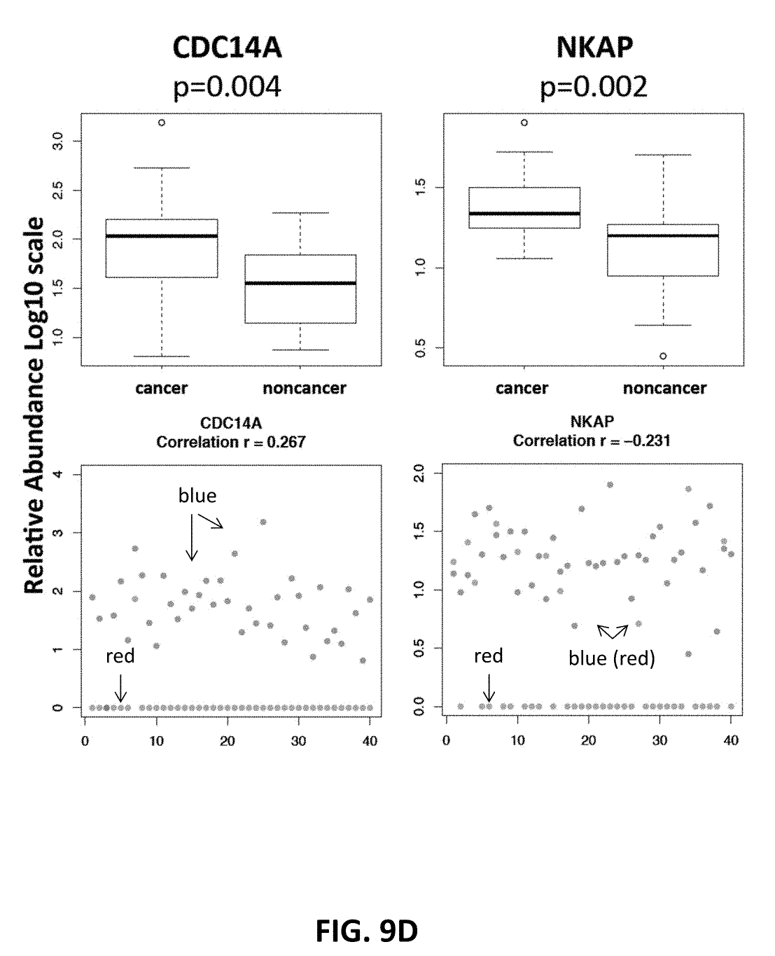

To illustrate that the signal detected in the plasma EVs for the selected features was not from the trace of leftover plasma, FIGS. 9A-D show some of those features with the most significant difference between advanced breast cancer and the non-cancer patients, as well as the comparison of their signals in plasma EVs to the corresponding neat plasma. Not only these features showed significantly higher relative abundance in advanced breast cancer group compared to the control group (p<0.05), but also they were mostly detected in the enriched plasma EVs but not the corresponding neat plasma. Little correlation was observed between the signals in enriched plasma EVs and the neat plasma, indicating the selected features were from the F68 enriched EVs instead from trace of the plasma leftover.

### DISCUSSION

Secreted EV encompasses a very rapidly growing scientific field in biology and medicine. To date, however, it is technically challenging to obtain a pure EV fraction free from non-vesicular components. It would be preferable to enrich the EV fraction with high efficiency while containing as less as possible of the plasma protein contaminations. Precipitation based EV isolation methods have advantages in efficiency, reproducibility and simplicity compared to classical UC or IP based methods. Precipitation methods also vary in such parameters. In this Example, we showed that the Pluronic copolymer F68 was able to enrich the plasma EVs ranging from 20 nm to 100 nm in size with intact membrane structure, and comprising a highly enriched set of EV-related proteins as well as varieties of RNA species. At the same time, the method provided herein more efficiently removed high abundant plasma proteins contaminations compared to prior art methods. The enriched EVs were shown to be compatible with a variety of downstream applications including TEM, FL and MS without requiring additional procedures to remove the copolymer. According to the minimal experimental requirements for definition of EVs, at least 3 proteins should be reported in at least semi-quantitative manner in any EV preparation [22]. Here we reported not only the higher enrichment of EV related protein, including CD9, ACTB, HSP70 and VWF, but also lower the potential high abundant protein contaminants (ALB, A2M, and IgGs) from plasma by two semi-quantitative methods. Shotgun proteome analysis further showed that our method enriched more potential EV proteins than alternate precipitation methods.

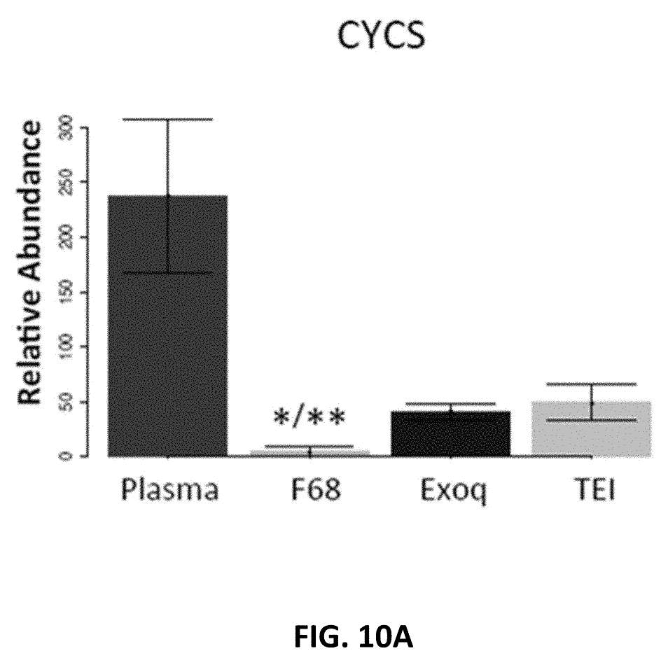

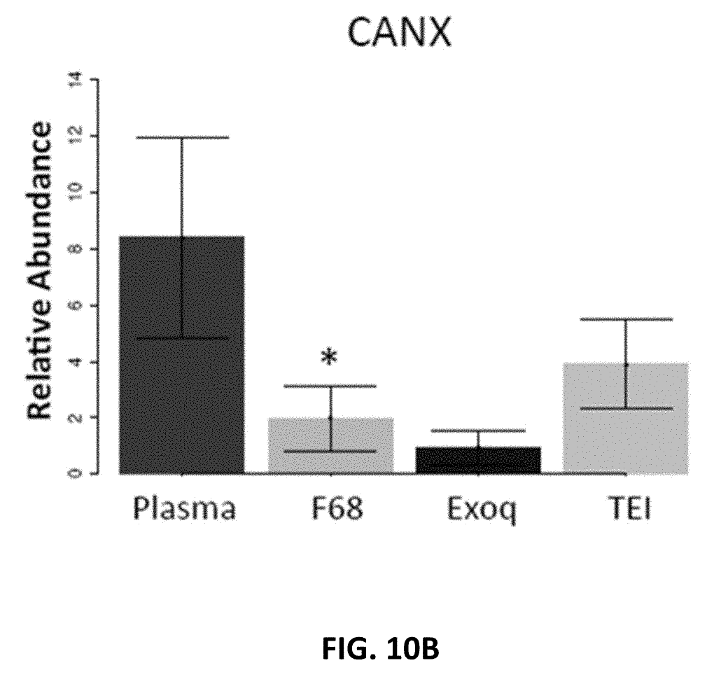

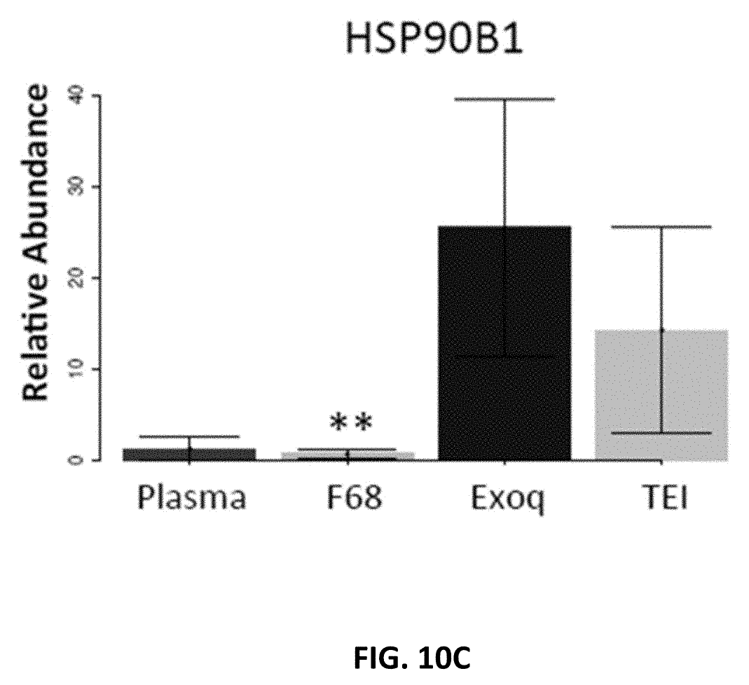

Levels of biomarkers that are believed to be absent in EVs can be used to evaluate the purity of the enriched EVs [22]. Semi-quantitative comparison for such markers were shown in FIGS. 10A-C, including cytochrome c (FIG. 10A; CYCS, mitochondria), calnexin (FIG. 10B; CANX, ER) and endoplasmin B1 (FIG. 10C; HSP90B1, ER). Compared to prior art methods, F68 precipitation was the only method that consistently showed significantly lower enrichment of these mitochondria and endoplasmic reticulum protein markers in both comparisons to neat plasma and to other methods.

Almost half of the proteins that were enriched by the F68 method (122 out of 263) were not matched to the categories described in FIGS. 5C-E. Most of these proteins belong to three sub-categories, including integral components of membrane, cytoplasm (cytosol), and nucleus proteins. The integral components of membrane includes those proteins that might involve in interaction with the membrane bound receptor signaling cascades, such as C—C chemokine receptor type 8 (CCR8), Cysteinyl leukotriene receptor 2 (CYSLTR2), Probable G-protein coupled receptor 151 (GPR151), ATP-binding cassette sub-family G member 5 (ABCGS). Several proteins were identified as cytoplasm/cytosol and nucleus proteins with RNA/DNA binding capability, including YTH domain-containing family protein 1 (YTHDF1), Cysteine/serine-rich nuclear protein 1 (CSRNP1), PHD finger protein 21A (PHF21A), RNA-binding protein 42 (RBM42), Zinc finger protein (ZFAT), Transducin-like enhancer protein 1 (TLE1), Homeobox-containing protein 1 (HMBOX1), Single-stranded DNA cytosine deaminase (AICDA), Krueppel-like factor 17 (KLF17), which are involved in varieties of functions from potential translation regulation, tumor suppressor to transcription factor activity. The finding is consistent to the study reporting that transcriptional regulator proteins were abundant in EVs [39]. These proteins are potential cargo payload within EVs.

Although a variety of RNA species have been found in EVs [32], profiling of RNA/smRNA from plasma EVs has been challenging. For example, the profile was highly dependent on the method used in preparing the EV fraction. This was further complicated by the fact that smRNAs are well-known to circulate in the bloodstream and other body fluids in a stable, cell-free status [40], resulting in RNA profiles that vary by different methods and may contains undistinguishable contaminants by various degrees. Our method described herein improves upon such studies by providing enriched EV related proteins with less contamination by highly abundant plasma proteins.

In a broader view, the RNA/smRNA profiles for plasma EVs enriched by F68 were consistent to other PEG-based enrichment methods from other studies (either from Exoquick or from TEI kit preparations) [32, 41]. miRNA, piRNA, tRNA, tRFs, Y_RNA, RNY, MtRNA, protein_coding RNA, rRNA, lincRNA as well as snRNA and snoRNA were all detectable. Interestingly, we detected high amount of Mt_RNA (FIG. 6B). Without being bound by theory, there might be two explanations: 1) small mitochondrial debris might still be co-precipitated with the copolymer, which is supported by the existence of small portion of particles with larger size, even in the UC cell line EV preparations; 2) alternatively, a recent study showed that mitochondria are also able to generate small vesicular carriers that transport mitochondrial contents to other intracellular organelles. One of the targets of such transportation from mitochondria was the late endosome, or multi-vesicular body (MVB), where the EVs were formed [42], suggesting communication between mitochondrial and endosome or MVB. Such communication might be a reason for high levels of mitochondrial RNA in the plasma EV fraction.

To explore the potential application for our enrichment method provided herein, proteomic profiling was performed to compare plasma EVs enriched from advanced breast cancer and non-cancer controls. The breast cancer plasma samples were all from advanced clinical stage (III and IV) based on two rational. First, according to the definition of breast cancer stages (www.cancer.net/cancer-types/breast-cancer/stages), a major character of clinical stage III and IV is that cancer of any size has spread to 4 to 9 axillary lymph nodes or has spread to the chest wall or caused swelling, or ulceration of the breast, as well as further spread to other organs. Such symptoms suggest cancer cells are actively spreading to distal tissue and maybe constantly in contact with the blood stream. Accordingly, cancer cell generated EVs may be more likely expelled into the blood stream. Second, EVs selected from the model were expected to be related to the cancer aggression, metastasis or invasion.

The protein feature selection process we employed also focused on those proteins in common between enriched plasma EVs and breast cancer cell line EVs. This weights the association of the observed features to EVs. In addition, EVs in plasma may come from variety of sources unrelated to breast cancer cells. By only using the features that could be detected from EVs generated by breast cancer cell lines, the chance of the selected features are associated with breast cancer is increased. Using such filters, the resulted features are more likely to associate with breast cancer.

Although genotypes, morphologies and growth rates may differ between breast cancer cell lines used in the Example, namely MCF7, MDA-MB-231, MDA-MB-468 and T47D [43], all of these cell lines are derived from cancer metastasis/invasion sites [44, 45, 46, 47] and all show aggression, metastasis and invasive behavior in different models [43, 48]. Without being bound by theory, common factors might be involved in such behavior. We set out to determine whether such common features could be identified in plasma EV and whether these features could be used to differentiate the cancer patient from the non-cancer patients.

In the LOO cross-validation model, 60 core protein features were selected (FIGS. 8A-C). These features were detected in both patient plasma EVs and the breast cancer cell lines generated EVs, and were able to separate the advanced cancer sample population from non-cancer patients using either the cross-validation model (FIG. 8B) or by un-supervised hierarchical cluster method (FIG. 8C). Interestingly, according to the information from The Human Protein Atlas database [49, 50, 51], as shown in Table 6, expression of over 70% of these features (43 out of 60) have been confirmed by immunohistochemistry on breast cancer tissues slides, consistent with our approach to identify common features from enriched plasma EVs with features from EVs generated by breast cancer cell lines.

TABLE 6
Summary of 60 Features selected from the LOO model

| | | IHC expression | References to | P value |
|---|---|---|---|---|
| Gene Name | Prognostic Value in Cancer | breast cancer tissue | Breast Cancer | (t-test) |
| ACTB | Renal, Head and Neck Cancer | Yes | [57] | 0.002 |
| ACTC1 | Head and Neck, Urothelial | Yes | [58] | 0.001 |
| | Cancer | | | |
| ANKRD62 | NA | NA | | 0.004 |
| APOE | NA | Yes | [59] | 0.019 |
| ATR | Pancreatic, Liver Cancer | NA | | 0.004 |
| CDC14A | NA | Yes | [52, 60, 61] | 0.004 |
| CFAP44 | Urothelial Cancer | NA | | 0.005 |
| CHD2 | NA | Yes | [62] | 0.009 |
| CHD7 | Endometrial Cancer | Yes | [63] | 0.02 |
| CNOT2 | Renal, Liver, Melanoma | Yes | [53, 64] | 0.003 |
| | Cancer | | | |
| EGFR | Urothelial Cancer | NA | [65] | 0.003 |
| HRC | NA | NA | | 0.003 |
| ITGB3 | NA | NA | [54, 55, 66, 67] | 0.017 |
| LRP1B | NA | Yes | | 0.002 |
| MTMR3 | NA | Yes | [68] | 0.009 |
| NKAP | Breast Cancer | Yes | [69, 70] | 0.002 |
| ODF2 | Liver, Prostate Cancer | Yes | | 0.001 |
| POTEE | NA | Yes | [71] | 0.008 |
| POTEKP | NA | NA | | 0.003 |
| PPIG | Renal Cancer | Yes | | 0.002 |
| PRSS3 | Endometrial Cancer | Yes | [72, 73, 74] | 0.003 |
| REV3L | NA | Yes | [75, 76] | 0.002 |
| SCN2A | NA | NA | [77] | 0.003 |
| SPTBN1 | Renal Cancer | Yes | [78, 79] | 0.003 |
| TMEM87A | NA | Yes | | 0.019 |
| XIRP2 | NA | Yes | | 0.013 |
| ZNF33B | NA | Yes | | 0.006 |
| ZNF804A | NA | Yes | | 0.002 |
| PRPF4B | Urothelial, Liver Cancer | Yes | | 0.006 |
| BAZ1B | NA | NA | | 0.004 |
| CCDC191 | Urothelial Cancer | Yes | | 0.013 |
| CNTLN | NA | Yes | | 0.011 |
| KIAA0100 | Liver Cancer | Yes | [56, 80] | 0.001 |
| NEB | NA | NA | | 0.024 |
| TRPM6 | NA | NA | | 0.017 |
| ZKSCAN5 | Liver, Thyroid Cancer | Yes | | 0.024 |
| ZC3H13 | renal Cancer | Yes | | 0.063 |
| DST | NA | Yes | [81, 82, 83] | 0.008 |
| EGLN1/PHD2 | Cervical Cancer | Yes | [84, 85, 86] | 0.026 |
| KDM5A | NA | NA | [87, 88, 89] | 0.02 |
| NCAPD3 | NA | NA | | 0.011 |
| NEXN | Colorectal Cancer | NA | | 0.011 |
| SETD2 | Renal, Melanoma Cancer | Yes | [90, 91] | 0.022 |
| ADAM17 | Urothelial Cancer | Yes | [92, 93, 94] | 0.043 |
| CACNA1B | NA | Yes | [95] | 0.035 |
| GOLGA6L2 | NA | NA | | 0.018 |
| IQCA1L | NA | NA | | 0.024 |
| TTN | NA | Yes | | 0.02 |
| F5 | Stomach Cancer | Yes | | 0.011 |
| EIF3A | NA | Yes | [96] | 0.019 |
| MYO6 | Renal Cancer | Yes | [97, 98] | 0.05 |
| CALD1 | Renal, Melanoma Cancer | Yes | [99] | 0.023 |
| NRAP | NA | Yes | | 0.058 |
| CREBBP | Renal Cancer | Yes | | 0.023 |
| CCDC136 | NA | NA | | 0.017 |
| MSL2 | Cervical Cancer | Yes | | 0.015 |
| KMT2A | NA | Yes | [100] | 0.056 |
| ZNF77 | Lung, Endometrial, Cervical | Yes | | 0.053 |
| | Cancer | | | |
| USP33 | NA | Yes | [101] | 0.044 |
| SORL1 | Renal Cancer | Yes | | 0.024 |

Also using The Human Protein Atlas database, the prognostic performance of these 60 features were also analyzed by respective RNAseq dataset generated by TCGA. The analysis was based on the FPKM value of each gene. Patients were classified into two expression groups and the correlation between expression level and patient survival was examined by Kaplan-Meier survival estimators, and the survival outcomes of the two groups were compared by log-rank tests (proteinatlas.org). Almost half of those protein (28 out of 60) features showed significant prognostic values on varieties of cancers respectively, implying their potential relevance to breast cancer. Thus, 28 out of 60 features show potential association to breast cancer aggression/metastasis/invasion (Table 6). For example, Dual specificity protein phosphatase (CDC14A) was shown to be recruited to the cell leading edge to regulate cell migration and adhesion in breast cancer cells [52]; CCR4-NOT transcription complex subunit 2 (CNOT2) was shown to promote proliferation and angiogenesis via VEGF signaling in breast cancer cells [53]; downregulation of Integrin beta-3 (ITGB3) modulates cell adhesion and invasion by interrupting Erk/Ets1 network in triple-negative breast cancer [54]; tumor but not stromal expression of ITGB3 is essential and required early bone-metastatic breast cancer [55]; and high levels of Antigen MLAA-22 (KIAA0100) expression were associated with poor prognosis in patients with invasive ductal carcinomas of the breast [56]. The results are consistent with our approach to identify protein features related to advanced tumors, including aggressive, metastatic and/or invasive characteristics.

### REFERENCES (NOTED BY BRACKETED NUMBERS ABOVE)

1. Orozco A F, Lewis D E. 2010; 77:502-14.
2. Stoorvogel W, et al. Traffic. 2002; 3:321-30.
3. Livshits M A, et al. Sci Rep. 2015; 5:17319.
4. Thery C, et al. Curr Protoc Cell Biol. 2006; Chapter 3:Unit 3 22.
5. U.S. Pat. No. 9,005,888.
6. Helwa I, et al. PLoS One. 2017; 12:e0170628.
7. Caradec J, et al. Clin Biochem. 2014; 47:1286-92.
8. Van Deun J, et al. 2014; 3: 24858-10.3402/jev.v3.24858.
9. Kabanov A V, et al. Adv Drug Deliv Rev. 2002; 54:223-33.
10. Shelat P B, et al. J Neurosci. 2013; 33:12287-99.
11. Firestone M A, et al. Biomacromolecules. 2003; 4:1539-49.
12. Lee R C, et al. Proc Natl Acad Sci USA. 1992; 89:4524-8.
13. Marks J D, et al. FASEB J. 2001; 15:1107-9.
14. Melnichenko A A, et al. 2012; 47:995-1000.
15. Schneider C A, et al. Nat Methods. 2012; 9:671-5.
16. Trapnell C, et al. Nat Biotechnol. 2010; 28:511-5.
17. Rekker K, et al. PLoS One. 2013; 8:e81166.
18. Chambers M C, et al. Nat Biotechnol. 2012; 30:918-20.
19. Kim S, Pevzner P A. Nat Commun. 2014; 5:5277.
20. Krey J F, et al. J Proteome Res. 2014; 13:1034-44.
21. Sardiu M E, Washburn M P. Nat Biotechnol. 2010; 28:40-2.
22. Lotvall J, et al. J Extracell Vesicles. 2014; 3:26913.
23. Ohshima K, et al. PLoS One. 2010; 5:e13247.
24. Hannafon B N, et al. Mol Cancer. 2015; 14:133.
25. Zhang J, et al. 2015; 13:17-24.
26. Hortin G L, et al. Clin Chem. 2008; 54:1608-16.
27. Thery C, et al. Nat Rev Immunol. 2002; 2:569-79.
28. Badimon L, et al. Front Pharmacol. 2016; 7:293.
29. Ge R, et al. Cancer Microenviron. 2012; 5:323-32.
30. Buschow S I, et al. Immunol Cell Biol. 2010; 88:851-6.
31. Keerthikumar S, et al. J Mol Biol. 2016; 428:688-92.
32. van Balkom B W, et al. J Extracell Vesicles. 2015; 4:26760.
33. Raposo G, Stoorvogel W. J Cell Biol. 2013; 200:373-83.
34. Hewson C, et al. Noncoding RNA Res. 2016; 1:3-11.
35. Khurana R, et al. RNA. 2017; 23:142-52.
36. Shore S, et al. PLoS One. 2016; 11:e0167009.
37. Venkatesh T, et al. Gene. 2016; 579:133-8.
38. Hizir Z, et al. Cell Death Dis. 2017; 8:e2530.
39. Ung T H, et al. Cancer Sci. 2014; 105:1384-92.
40. Liang H, et al. Wiley Interdiscip Rev RNA. 2014; 5:285-300.
41. Huang X, et al. BMC Genomics. 2013; 14:319.
42. Sugiura A, et al. EMBO J. 2014; 33:2142-56.
43. Holliday D L, Speirs V. Breast Cancer Res. 2011; 13:215.
44. Soule H D, et al. J Natl Cancer Inst. 1973; 51:1409-16.
45. Brinkley B R, et al. Cancer Res. 1980; 40:3118-29.
46. Cailleau R, et al. In Vitro. 1978; 14:911-5.
47. Keydar I, et al. Eur J Cancer. 1979; 15:659-70.
48. Fantozzi A, Christofori G. Breast Cancer Res. 2006; 8:212.
49. Uhlen M, et al. Proteomics. Science. 2015; 347:1260419.
50. Uhlen M, et al. Nat Biotechnol. 2010; 28:1248-50.
51. Uhlen M, et al. Mol Cell Proteomics. 2005; 4:1920-32.
52. Chen N P, et al. Proc Natl Acad Sci USA. 2016; 113:990-5.
53. Sohn E J, et al. Cancer Lett. 2017; 412:88-98.
54. Li W, et al. Int J Oncol. 2016; 48:1155-64.
55. Carter R Z, et al. J Pathol. 2015; 235:760-72.
56. Liu T, et al. PLoS One. 2014; 9:e86952.
57. Oliveira N C, et al. Genet Mol Res. 2016; 15.
58. Li Y, et al. Oncol Lett. 2017; 13:1856-60.
59. Liu Y L, et al. ThOnco Targets Ther. 2016; 9:1241-9.
60. Stevens K N, et al. Breast Cancer Res Treat. 2011; 129:617-22.
61. Lucci M A, et al. Cell Oncol. 2010; 32:361-72.
62. Russo J, Russo I H. Horm Mol Biol Clin Investig. 2012; 9:3-10.
63. Chu X, et al. Mol Oncol. 2017; 11:1348-60.
64. Faraji F, et al. Genome Res. 2014; 24:227-40.
65. Gonzalez-Conchas G A, et al. Cancer Treat Rev. 2017; 62:1-8.
66. Qian W, et al. J Vasc Res. 2015; 52:197-209.
67. Chi Y, et al. BMC Cancer. 2014; 14:577.
68. Gong Y, et al. Sci Rep. 2015; 5:11650.
69. Marcotte R, et al. Cell. 2016; 164:293-309.
70. Abdelkarim M, et al. PLoS One. 2011; 6:e23334.
71. Cine N, et al. Oncol Rep. 2014; 32:1078-86.
72. Qian L, et al. Oncotarget. 2017; 8:21444-53.
73. Ma R, et al. Gynecol Oncol. 2015; 137:546-52.
74. Hockla A, et al. Breast Cancer Res Treat. 2010; 124:27-38.
75. Yang L, et al. PLoS One. 2015; 10:e0120334.
76. Varadi V, et al. Breast Cancer Res Treat. 2011; 129:235-45.
77. Roger S, et al. Front Pharmacol. 2015; 6:152.
78. Gau D M, et al. Cell Cycle. 2015; 14:1884-92.
79. Gur-Dedeoglu B, et al. BMC Cancer. 2008; 8:396.
80. Song J, et al. Biochim Biophys Acta. 2006; 1760:62-9.
81. Schuetz C S, et al. Cancer Res. 2006; 66:5278-86.
82. Lee S, et al. Cancer Res. 2012; 72:4574-86.
83. Michael M, et al. J Invest Dermatol. 2014; 134:773-82.
84. Kuchnio A, et al. Oncotarget. 2015; 6:35149-50.
85. Bordoli M R, et al. Oncogene. 2011; 30:548-60.
86. Singh M, et al. Breast Cancer. 2016; 23:820-9.
87. Paolicchi E, et al. Crit Rev Oncol Hematol. 2013; 86:97-103.
88. Hou J, et al Q. Am J Transl Res. 2012; 4:247-56.
89. Gale M, et al. Oncotarget. 2016; 7:39931-44.
90. Newbold R F, Mokbel K. Anticancer Res. 2010; 30:3309-11.
91. Al Sarakbi W, et al. BMC Cancer. 2009; 9:290.
92. Shen H, Li L, et al. Tumour Biol. 2016. 37: 15359-15370.
93. Narita D, et al. Pathol Oncol Res. 2011; 17:375-85.
94. Zheng X, et al. Cancer Biol Ther. 2009; 8:1045-54.
95. Wang C Y, et al.. PLoS One. 2015; 10:e0125766.
96. Yamaga R, et al. Horm Cancer. 2013; 4:222-32.
97. Bjorklund S S, et al. Sci Rep. 2017; 7:5568.
98. Wang H, et al. 2015; 30:330-5.
99. De Marchi T, et al. Oncotarget. 2016; 7:3098-110.
100. Zou J X, et al. Mol Cancer Res. 2014; 12:539-49.
101. Yuasa-Kawada J, et al. Proc Natl Acad Sci USA. 2009; 106:14530-5.

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of isolating at least one extracellular vesicle from a sample comprising:
(a) contacting the sample with at least one pluronic block copolymer; and
(b) separating extracellular vesicles associated with the pluronic block copolymers from one or more components of the sample thereby isolating the at least one extracellular vesicle.

2. The method of claim 1, wherein the at least one pluronic block copolymer comprises F68, F127 or HLB0.80.

3. The method of claim 1, wherein the at least one extracellular vesicle comprises vesicles having a diameter between 10 nm and 1000 nm.

4. The method of claim 1, wherein the at least one extracellular vesicle comprises vesicles having a diameter between 20 nm and 200 nm.

5. The method of claim 1, wherein the at least one extracellular vesicle comprises vesicles having a diameter between 20 nm and 100 nm.

6. The method of claim 1, further comprising subjecting the at least one extracellular vesicle to affinity purification, filtration, polymer precipitation, PEG precipitation, ultracentrifugation, a molecular crowding reagent, affinity isolation, affinity selection, or any combination thereof.

7. The method of claim 1, wherein the sample comprises a bodily fluid, tissue sample or cell culture.

8. The method of claim 6, wherein the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair oil, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood.

9. (canceled)

10. The method of claim 1, further comprising selectively depleting at least one contaminant from the sample prior to step (a), between steps (a) and (b), after step (b), or any combination thereof.

11. The method of claim 10, wherein the at least one contaminant comprises at least one highly abundant protein.

12. The method of claim 1, further comprising detecting at least one surface antigen associated with the isolated at least one extracellular vesicle.

13. The method of claim 12, wherein the at least one surface antigen is selected from Table 3 or Table 6.

14. The method of claim 1, further comprising contacting the isolated at least one extracellular vesicle with at least one binding agent, wherein the at least one binding agent comprises a nucleic acid, DNA molecule, RNA molecule, antibody, antibody fragment, aptamer, peptoid, zDNA, peptide nucleic acid (PNA), locked nucleic acid (LNA), lectin, peptide, dendrimer, membrane protein labeling agent, chemical compound, or a combination thereof.

15. (canceled)

16. The method of claim 1, further comprising:
(c) contacting the isolated at least one extracellular vesicle with an aptamer library prior to step (a), between steps (a) and (b), after step (b), or any combination thereof; and
(d) identifying the members of the aptamer library that bound the isolated at least one extracellular vesicle.

17. The method of claim 16, wherein the identifying comprises sequencing, hybridization or amplification.

18. The method of claim 16, further comprising detecting at least one payload biomarker within the isolated at least one extracellular vesicle.

19. The method of claim 18, wherein the one or more payload biomarker comprises at least one nucleic acid, peptide, protein, lipid, antigen, carbohydrate, and/or proteoglycan.

20. The method of claim 19, wherein the nucleic acid comprises at least one of DNA, RNA, mRNA, smRNA, microRNA, Y RNA, lincRNA, mitochondrial RNA, snoRNA, snRNA, rRNA, tRNA, siRNA, hnRNA, or shRNA.

21. (canceled)

22. (canceled)

23. The method of claim 16, wherein the sample is from a subject suspected of having or being predisposed to a disease or disorder.

24. The method of claim 23, wherein the disease or disorder comprises a cancer.

25.-45. (canceled)
